(19) United States
(12) Patent Application Publication
(10) Pub. No.: US 2024/0229144 A1
(43) Pub. Date: Jul. 11, 2024

(54) METHODS FOR DETECTING OR TREATING GLIOBLASTOMA MULTIFORME

(71) Applicant: BAYLOR RESEARCH INSTITUTE, Dallas, TX (US)

(72) Inventors: Erxi Wu, Dallas, TX (US); Dan Qi, Dallas, TX (US); Ekokobe Fonkem, Dallas, TX (US); Jason Huang, Dallas, TX (US)

(73) Assignee: BAYLOR RESEARCH INSTITUTE, Dallas, TX (US)

(21) Appl. No.: 18/268,857

(22) PCT Filed: Dec. 21, 2021
(86) PCT No.: PCT/US2021/073063

§ 371 Date: Jun. 21, 2023

Related U.S. Application Data
(60) Provisional application No. 63/129,387, filed on Dec. 22, 2020.

(51) Int. Cl. C12Q 1/6886; C12Q 1/6851; C12Q 1/6874

(52) U.S. Cl. CPC C12Q 1/6886; C12Q 1/6851; C12Q 1/6874; C12Q 2600/158; C12Q 2600/178

(57) ABSTRACT

The current disclosure fulfills a need in the art by providing biomarkers that can be used in more effective diagnostic and C treatment methods for GBM patients. Accordingly, aspects of the disclosure relate to a method for treating a subject with glioblastoma multiforme (GBM), the method comprising treating the subject for GBM after the expression level of one or more biomarkers has been determined in a sample from the subject. Further aspects relate to a method for prognosing and/or diagnosing a subject for GBM comprising: a) measuring the level of expression of one or more biomarkers in a sample from the subject; b) comparing the level(s) of expression to a control sample(s) or control level(s) of expression; and, c) prognosing and/or diagnosing the subject based on the levels of measured expression.

### CROSS REFERENCE TO RELATED APPLICATIONS

This application claims the benefit of priority to U.S. Provisional Patent Application Ser. No. 63/129,387, filed Dec. 22, 2020 hereby incorporated by reference in its entirety.

### FIELD OF THE INVENTION

The present invention relates generally to the fields of molecular biology and therapeutic diagnosis.

### BACKGROUND

Glioblastoma multiforme (GBM) is the most aggressive and heterogeneous type of brain cancer, aka malignant brain glioma (WHO grade IV) which occurs in the human central nervous system (CNS) with a poor survival. Despite multidisciplinary treatments such as surgery, chemotherapy, and radiotherapy, the median survival time for patients with GBM ranges in median between 12 and 15 months. The diagnosis, response monitoring of therapeutic interventions, and clinical assessment of GBM prognosis often rely on imaging techniques, and surgical brain biopsy, which is highly invasive and sometimes only partially reflect the disease status due to limited tissue pieces. Despite advanced marker discovery techniques using liquid biopsies, such as blood and cerebrospinal fluid, for the detection of circulating tumor cells (CTCs), cell-free nucleic acids and extracellular vesicles, there are still few effective GBM specific liquid biomarkers, especially blood-derived markers, available in clinical settings to reduce the necessity of multiple invasive brain biopsies and to assist the application of imaging diagnosis and surveillance. Molecular characteristics identified from GBM tumor tissue including methylation of O⁶-methylguanine-DNA methyltransferase (MGMT) gene promoter region, mutation of isocitrate dehydrogenase (IDH), alteration (such as amplification) of epidermal growth factor receptor (EGFR), and abnormalities of chromosome (for example, 1p/19q codeletion) are the few markers currently used in GBM diagnosis and prognosis. GBM patients with MGMT methylation usually respond better to temozolomide treatment. Patients with IDH mutations appear to have a better outcome than patients with IDH wildtype; however, most primary GBMs are IDH wildtype 18-20. To help improve the survival of GBM patients, minimally invasive and ultrasensitive biomarkers or biomarker signature as indicators for disease diagnosis, therapy responses are recognized as essential tools in pressing need.

### SUMMARY

The current disclosure fulfills a need in the art by providing biomarkers that can be used in more effective diagnostic and treatment methods for GBM patients. Accordingly, aspects of the disclosure relate to a method for treating a subject with glioblastoma multiforme (GBM), the method comprising treating the subject for GBM after the expression level of one or more biomarkers selected from MMP9, TMEM92, C1orf226, CD163, LINC00482, miR-3918, AK5, CCR7, CD200, MICU3, miR-760, SH3PXD2B, miR-760, miR-125b-5p, miR-1299, a miRNA biomarker listed in FIG. 3C or 12B, and a gene biomarker listed in FIG. 3D, 4C, or 12B has been determined in a sample from the subject. Further aspects relate to a method for evaluating a subject comprising measuring the level of expression of one or more biomarkers selected from MMP9, TMEM92, C1orf226, CD163, LINC00482, miR-3918, AK5, CCR7, CD200, MICU3, miR-760, SH3PXD2B, miR-760, miR-125b-5p, miR-1299, a miRNA biomarker listed in FIG. 3C or 12B, and a gene biomarker listed in FIG. 3D, 4C, or 12B in a sample from the subject. Further aspects relate to a method of prognosing and/or diagnosing a subject for GBM comprising: a) measuring the level of expression of one or more of MMP9, TMEM92, C1orf226, CD163, LINC00482, miR-3918, AK5, CCR7, CD200, MICU3, miR-760, SH3PXD2B, miR-760, miR-125b-5p, miR-1299, a miRNA biomarker listed in FIG. 3C or 12B, and a gene biomarker listed in FIG. 3D, 4C, or 12B in a sample from the subject; b) comparing the level(s) of expression to a control sample(s) or control level(s) of expression; and, c) prognosing and/or diagnosing the subject based on the levels of measured expression.

Also described is a kit comprising 1, 2, 3, 4, or 5 detection agents for determining expression levels of biomarkers for GBM, wherein the biomarkers comprise one or more MMP9, TMEM92, C1orf226, CD163, LINC00482, miR-3918, AK5, CCR7, CD200, MICU3, miR-760, SH3PXD2B, miR-760, miR-125b-5p, miR-1299, a miRNA biomarker listed in FIG. 3C or 12B, and a gene biomarker listed in FIG. 3D, 4C, or 12B. Further aspects relate to a method for making and amplifying cDNA comprising a) reverse transcribing the mRNA in a biological sample from a subject; and b) contacting the sample from a with primers to amplify one or more biomarkers, wherein the biomarkers comprise one or more of MMP9, TMEM92, C1orf226, CD163, LINC00482, miR-3918, AK5, CCR7, CD200, MICU3, miR-760, SH3PXD2B, miR-760, miR-125b-5p, miR-1299, a miRNA biomarker listed in FIG. 3C or 12B, and a gene biomarker listed in FIG. 3D, 4C, or 12B.

In some aspects the biomarker comprises one or more of MMP9, TMEM92, C1orf226, CD163, LINC00482, miR-3918, AK5, CCR7, CD200, MICU3, and miR-760. In some aspects, the biomarker comprises one or more of MMP9, TMEM92, C1orf226, CD163, LINC00482, hsa-miR-3918, AK5, CD200, MICU3, and hsa-miR-760. In some aspects, the biomarker comprises or consists of MMP9, TMEM92, C1orf226, CD163, LINC00482, hsa-miR-3918, AK5, CD200, MICU3, and hsa-miR-760.

The sample may be further defined as a globin mRNA depleated sample. In some aspects, the methods comprise or further comprise depleting globin mRNA from the sample. In some aspects, the globin mRNA is depleted prior to reverse transcription. A globin mRNA depleated sample refers to one that has had globin mRNA removed. Such methods are known in the art. For example, the GLOBINclear™ Kit (Ambion, Austin, Texas) may be used to remove the highly abundant hemoglobin mRNA. As an example, total RNA from a sample may be hybridized with a biotinylated Capture OLIGO Mix that is specific for human mRNA hemoglobin a and 3. Streptavidin Magnetic Beads can then be added to bind the biotinylated oligonucleotides that hybridized with globin mRNA and then were pulled down by magnet. The globin mRNA depleted RNA can then be transferred to a fresh tube and further purified with a rapid magnetic bead-based purification process. The sample can then be processed to determine the levels of the biomarkers.

The sample may be a the sample from the subject may comprise a biopsy sample, a serum sample, a tissue sample, a blood sample, a whole blood sample, or a plasma sample. In some aspects, the sample comprises the sample comprises a blood sample, a whole blood sample, or a plasma sample. The normal tissues may comprise non-cancerous neural tissues. In some aspects, the sample from the subject comprises nucleic acids. In some aspects, the sample from the subject comprises a fractionated blood sample comprising nucleic acids. In some aspects, the sample comprises a sample from the human. In some aspects, the subject is a human subject. The subject may be one that has not been diagnosed with or has not been treated for GBM. In some aspects, the subject has not been identified as high risk for GBM. In some aspects, the subject has been diagnosed or identified as high risk for GBM.

In some aspects, at least MMP9 was determined, evaluated, or measured in a sample from the subject. In some aspects, at least TMEM92 was determined, evaluated, or measured in a sample from the subject. In some aspects, at least C1orf226 was determined, evaluated, or measured in a sample from the subject. In some aspects, at least CD163 was determined, evaluated, or measured in a sample from the subject. In some aspects, at least LINC00482 was determined, evaluated, or measured in a sample from the subject. In some aspects, at least miR-3918 was determined, evaluated, or measured in a sample from the subject. In some aspects, at least AK5 was determined, evaluated, or measured in a sample from the subject. In some aspects, at least CCR7 was determined, evaluated, or measured in a sample from the subject. In some aspects, at least CD200 was determined, evaluated, or measured in a sample from the subject. In some aspects, at least MICU3 was determined, evaluated, or measured in a sample from the subject. In some aspects, at least miR-760 was determined, evaluated, or measured in a sample from the subject. The biomarker may be further described as a human gene or a human miRNA. In some aspects, the biomarker is measured pre-operative or before surgical resection of the GBM tumor. In some aspects, the biomarker is measured post-operative or after surgical resection of the GBM tumor. In some aspects, the expression level of no other biomarker in the biological sample was measured, evaluated, or determined in the sample. The subject may be one that has undergone surgery to resect all or part of the cancer. In some aspects, the subject has not undergone surgical resection of the tumor. In some aspects, the subject has not been diagnosed with or has not been treated for GBM.

In some aspects, the expression levels of the one or more biomarkers in the sample was determined to be, evaluated as, or measured as i) increased compared to the levels of expression in samples from subjects identified as not having GBM, subjects identified as low risk, or normal tissues or ii) within the range of expression levels in samples of subjects identified as having GBM or identified as high risk. In some aspects, the expression levels of the one or more biomarkers in the sample was determined to be, evaluated as, or measured as i) decreased compared to the levels of expression in samples from subjects identified as not having GBM, subjects identified as low risk, or normal tissues or ii) within the range of expression levels in samples of subjects identified as having GBM or identified as high risk. In some aspects, the expression levels of at least one of the biomarkers in the sample was determined to be, evaluated as, or measured as i) increased compared to the levels of expression in samples from subjects identified as not having GBM, subjects identified as low risk, or in normal tissues or ii) within the range of expression levels in samples of subjects identified as having GBM or identified as high risk. In some aspects, the expression levels of at least one of the biomarkers in the sample was determined to be, evaluated as, or measured as i) decreased compared to the levels of expression in samples from subjects identified as not having GBM, subjects identified as low risk, or in normal tissues or ii) within the range of expression levels in samples of subjects identified as having GBM or identified as high risk. In some aspects, the expression levels of at least two of the biomarkers in the sample were determined to be, evaluated as, or measured as i) increased compared to the levels of expression in samples from subjects identified as not having GBM, subjects identified as low risk, or normal tissues or ii) within the range of expression levels in samples of subjects identified as having GBM or identified as high risk. In some aspects, the expression levels of at least two of the biomarkers in the sample were determined to be, evaluated as, or measured as i) decreased compared to the levels of expression in samples from subjects identified as not having GBM, subjects identified as low risk, or normal tissues or ii) within the range of expression levels in samples of subjects identified as having GBM or identified as high risk. In some aspects, the expression levels of at least three of the biomarkers in the sample were determined to be, evaluated as, or measured as i) increased compared to the levels of expression in samples from subjects identified as not having GBM, subjects identified as low risk, or in normal tissues or ii) within the range of expression levels in samples of subjects identified as having GBM or identified as high risk. In some aspects, the expression levels of at least three of the biomarkers in the sample were determined to be, evaluated as, or measured as i) increased compared to the levels of expression in samples from subjects identified as not having GBM, subjects identified as low risk, or in normal tissues or ii) within the range of expression levels in samples of subjects identified as having GBM or identified as high risk. In some aspects, the expression levels of at least four of the biomarkers in the sample were determined to be, evaluated as, or measured as i) increased compared to the levels of expression in samples from subjects identified as not having GBM, subjects identified as low risk, or in normal tissues or ii) within the range of expression levels in samples of subjects identified as having GBM or identified as high risk. In some aspects, the expression levels of at least four of the biomarkers in the sample were determined to be, evaluated as, or measured as i) decreased compared to the levels of expression in samples from subjects identified as not having GBM, subjects identified as low risk, or in normal tissues or ii) within the range of expression levels in samples of subjects identified as having GBM or identified as high risk. In some aspects, the expression levels of at least five of the biomarkers in the sample were determined to be, evaluated as, or measured as i) increased compared to the levels of expression in samples from subjects identified as not having GBM, subjects identified as low risk, or in normal tissues or ii) within the range of expression levels in samples of subjects identified as having GBM or identified as high risk. In some aspects, the expression levels of at least five of the biomarkers in the sample were determined to be, evaluated as, or measured as i) decreased compared to the levels of expression in samples from subjects identified as not having GBM, subjects identified as low risk, or in normal tissues or ii) within the range of expression levels in samples of subjects identified as having GBM or identified as high risk. In some aspects, the expression levels of at least six of the biomarkers in the sample were determined to be, evaluated as, or measured as i) increased compared to the levels of expression in samples from subjects identified as not having GBM, subjects identified as low risk, or in normal tissues or ii) within the range of expression levels in samples of subjects identified as having GBM or identified as high risk. In some aspects, the expression levels of at least six of the biomarkers in the sample were determined to be, evaluated as, or measured as i) decreased compared to the levels of expression in samples from subjects identified as not having GBM, subjects identified as low risk, or in normal tissues or ii) within the range of expression levels in samples of subjects identified as having GBM or identified as high risk. In some aspects, the expression levels of at least seven of the biomarkers in the sample were determined to be, evaluated as, or measured as i) increased compared to the levels of expression in samples from subjects identified as not having GBM, subjects identified as low risk, or in normal tissues or ii) within the range of expression levels in samples of subjects identified as having GBM or identified as high risk. In some aspects, the expression levels of at least seven of the biomarkers in the sample were determined to be, evaluated as, or measured as i) decreased compared to the levels of expression in samples from subjects identified as not having GBM, subjects identified as low risk, or in normal tissues or ii) within the range of expression levels in samples of subjects identified as having GBM or identified as high risk. In some aspects, the expression levels of at least eight of the biomarkers in the sample were determined to be, evaluated as, or measured as i) increased compared to the levels of expression in samples from subjects identified as not having GBM, subjects identified as low risk, or in normal tissues or ii) within the range of expression levels in samples of subjects identified as having GBM or identified as high risk. In some aspects, the expression levels of at least eight of the biomarkers in the sample were determined to be, evaluated as, or measured as i) decreased compared to the levels of expression in samples from subjects identified as not having GBM, subjects identified as low risk, or in normal tissues or ii) within the range of expression levels in samples of subjects identified as having GBM or identified as high risk. In some aspects, the expression levels of at least nine of the biomarkers in the sample were determined to be, evaluated as, or measured as i) increased compared to the levels of expression in samples from subjects identified as not having GBM, subjects identified as low risk, or in normal tissues or ii) within the range of expression levels in samples of subjects identified as having GBM or identified as high risk. In some aspects, the expression levels of at least nine of the biomarkers in the sample were determined to be, evaluated as, or measured as i) decreased compared to the levels of expression in samples from subjects identified as not having GBM, subjects identified as low risk, or in normal tissues or ii) within the range of expression levels in samples of subjects identified as having GBM or identified as high risk. In some aspects, the expression levels of at least ten of the biomarkers in the sample were determined to be, evaluated as, or measured as i) increased compared to the levels of expression in samples from subjects identified as not having GBM, subjects identified as low risk, or in normal tissues or ii) within the range of expression levels in samples of subjects identified as having GBM or identified as high risk. In some aspects, the expression levels of at least ten of the biomarkers in the sample were determined to be, evaluated as, or measured as i) decreased compared to the levels of expression in samples from subjects identified as not having GBM, subjects identified as low risk, or in normal tissues or ii) within the range of expression levels in samples of subjects identified as having GBM or identified as high risk. In some aspects, the expression levels of at least eleven of the biomarkers in the sample were determined to be, evaluated as, or measured as i) increased compared to the levels of expression in samples from subjects identified as not having GBM, subjects identified as low risk, or in normal tissues or ii) within the range of expression levels in samples of subjects identified as having GBM or identified as high risk. In some aspects, the expression levels of at least eleven of the biomarkers in the sample were determined to be, evaluated as, or measured as i) decreased compared to the levels of expression in samples from subjects identified as not having GBM, subjects identified as low risk, or in normal tissues or ii) within the range of expression levels in samples of subjects identified as having GBM or identified as high risk. In some aspects, the expression levels of MMP9, TMEM92, C1orf226, CD163, LINC00482 and/or miR-3918 were determined to be, evaluated as, or measured as i) increased compared to the levels of expression in samples from subjects identified as not having GBM, subjects identified as low risk, or in normal tissues or ii) within the range of expression levels in samples of subjects identified as having GBM or identified as high risk. In some aspects, the expression levels of AK5, CCR7, CD200, MICU3, and/or miR-760 were determined to be, evaluated as, or measured as i) decreased compared to the levels of expression in samples from subjects identified as not having GBM, subjects identified as low risk, or in normal tissues or ii) within the range of expression levels in samples of subjects identified as having GBM or identified as high risk.

In some aspects, the method comprises or further comprises treating the subject for GBM. The subject may be one that is treated after measuring expression of the biomarker. The treatment may comprise or further comprise one or more of anticonvulsants, corticosteroids, immunotherapy, surgery, radiotherapy, or chemotherapy. In some aspects, the chemotherapy comprises temozolomide. The chemotherapy may be administered orally or intravenously. In some aspects, the treatment in the methods of the disclosure may be a treatment described herein, such as immunostimulators, immunotherapies, dendritic cell therapy, CAR-T cell therapy, cytokine therapy, oncolytic virus, polysaccharides, neoantigens, chemotherapies, radiotherapy, and/or surgery.

In some aspects, the samples from subjects identified as not having GBM or identified as low risk comprises the level of expression of the one or more biomarkers in a blood sample or samples from subjects without GBM. The methods may comprise or further comprise comparing the level(s) of expression to a control sample(s) or control level(s) of expression. The control sample(s) may have expression levels that are representative of expression levels in samples from subjects identified as low risk or of subjects not having GBM. In some aspects, the control levels(s) comprise the levels of expression of the one or more biomarkers in non-cancerous neural tissues. In some aspects, the control sample(s) have expression levels that are representative of expression levels in samples from subjects identified as high risk or of subjects having GBM.

In some aspects, the subject is diagnosed as having GBM, prognosed as high risk, and/or treated when the expression levels of MMP9, TMEM92, C1orf226, CD163, LINC00482 and/or miR-3918 were determined to be i) increased compared to the levels of expression in samples from subjects identified as not having GBM, subjects identified as low risk, or in normal tissues or ii) within the range of expression levels in samples of subjects identified as having GBM or identified as high risk. In some aspects, the subject is diagnosed as having GBM, prognosed as high risk, and/or treated when the expression levels of AK5, CCR7, CD200, MICU3, and/or-miR-760 were determined to be i) decreased compared to the levels of expression in samples from subjects identified as not having GBM, subjects identified as low risk, or in normal tissues or ii) within the range of expression levels in samples of subjects identified as having GBM or identified as high risk. In some aspects, the subject is diagnosed as not having GBM, prognosed as low risk, and/or not treated when the expression levels of the one or more of MMP9, TMEM92, C1orf226, CD163, LINC00482 and/or miR-3918 in the subject were determined to be i) within range of levels of expression in samples from subjects identified as not having GBM, subjects identified as low risk, or in normal tissues or ii) decreased compared to expression levels in samples of subjects identified as having GBM or identified as high risk. In some aspects, the subject is diagnosed as not having GBM, prognosed as low risk, and/or not treated when the expression levels of the one or more of AK5, CCR7, CD200, MICU3, and/or-miR-760 in the subject were determined to be i) within range of levels of expression in samples from subjects identified as not having GBM, subjects identified as low risk, or in normal tissues or ii) increased compared to expression levels in samples of subjects identified as having GBM or identified as high risk.

Kits of the disclosure may comprise detection agent for determining expression levels of one or more of MMP9, TMEM92, C1orf226, CD163, LINC00482, miR-3918, AK5, CD200, MICU3, and miR-760. In some aspects, the kits comprise detection agent for determining expression levels of one or more of TMEM92, C1orf226, AK5, MICU3, and miR-3918. In some aspects, the kit comprises detection agents for determining expression levels of MMP9, TMEM92, C1orf226, CD163, LINC00482, miR-3918, AK5, CD200, MICU3, and miR-760. In some aspects, the kit further comprises one or more negative or positive control samples and/or control detection agents. The kit may also comprise globin reduction reagents. In some aspects, the kit comprises hemoglobin alpha and beta capture oligos.

Methods and kits are provided that concern the detection and/or treatment of glioblastoma multiforme. The claims set forth embodiments of these methods and kits. A list of biomarkers is provided in the claims. It is specifically contemplated that 1, 2, 3, 4, 5, 6, 7, 8, 9, 10 or more of any specific biomarker may be excluded in an embodiment described herein.

Throughout this application, the term “about” is used according to its plain and ordinary meaning in the area of cell and molecular biology to indicate that a value includes the standard deviation of error for the device or method being employed to determine the value.

The use of the word “a” or “an” when used in conjunction with the term “comprising” may mean “one,” but it is also consistent with the meaning of “one or more,” “at least one,” and “one or more than one.”

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

The words “comprising” (and any form of comprising, such as “comprise” and “comprises”), “having” (and any form of having, such as “have” and “has”), “including” (and any form of including, such as “includes” and “include”), “characterized by” (and any form of including, such as “characterized as”), or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments described in the context of the term “comprising” may also be implemented in the context of the term “consisting of” or “consisting essentially of.”

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments discussed elsewhere in a different Example or elsewhere in the application, such as in the Summary of Invention, Detailed Description of the Embodiments, Claims, and description of Figure Legends.

Any method disclosed herein may also be implemented as the use of a composition for treatment objectives.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: Overall experiment design and human subjects workflow. Figure produced partially with the help of Servier Medical Art.

FIG. 2A-D: Globin reduction increases sequencing sensitivity. A, Bar plots showing the number of mRNA reads for samples before and after GR. The red segments represent reads from globin genes. B, Bar plots showing the number of differentially expressed genes surviving FDR≤0.05 before and after GR. The red segments represent up-regulated genes, while blue represent down-regulated. C, Venn diagram showing the overlap of differentially expressed genes surviving FDR≤0.05 before and after GR. D, Sample level scatterplots comparing log 2 normalized counts before and after GR, with post-GR samples plotted along the x-axis and pre-GR samples along the y-axis. The red lines have slope=1 and pass through the origin, and the red dots represent globin genes. Pearson's correlation for each scatterplot is shown.

FIG. 3A-D: Gene, miRNA expression profiling and integrated functional network analysis. Bar graphs showing the top 12 KEGG pathways A and top 20 GO BP terms B identified by DAVID Functional Annotation on the predicted miRNA target genes overlapping with blood RNA-seq result. The x-axis is −log 10 p-value in blue for each pathway. Red dotted lines represent the −log 10 transformed p-values of 0.05. C, Heatmap of log 2 normalized counts for miRNAs that survived P≤0.05 and |log 2FC|≥1.0. For each miRNA, the mean of the control group was subtracted, and then hierarchical clustering was performed. D, Network connecting DEMs to differentially expressed target genes to identified KEGG pathways and GO terms. Three up-regulated and four down-regulated miRNAs were focused and network connecting their predicted target DEGs was generated using Cytoscape. miRNAs are represented by triangles, genes by circles, KEGG terms by squares, and GO BP terms by diamonds.

FIG. 4A-C: Gene expression difference enrichment analysis. A, Venn diagram of concordant DEGs from Blood RNA-seq data and TCGA tissue data at FDR≤0.05. B, DEG enrichment analysis using Baylor Module Method. C, Heatmap of 90 narrow-downed DEGs in GBM blood samples from comprehensive analysis.

FIG. 5A-B: RT-qPCR validation of selected genes and miRNAs in newly collected blood samples and tissue samples. A, Mean values of multiple PCR results from every blood sample were plotted using Prism 7.0. B, Mean values of multiple PCR results from every tissue sample were plotted using Prism 7.0.

FIG. 6: 11 GBM related genes as potential blood biomarkers. Heatmap was generated using the means of the PCR results for blood samples with detailed patient clinical information.

FIG. 7A-B: RT-qPCR results of selected marker candidates in newly collected blood samples and tissue samples. A, Relative expressions were calculated from at least triplicate PCR results for each gene or miRNA. Data were represented as mean±standard derivation (SD) in percentages. B, Relative expressions were calculated from at least triplicate PCR results for each gene or miRNA. Data were represented as mean±standard derivation (SD) in percentages.

FIG. 8A-C: Expression comparison of selected genes using TCGA GBM transcriptome profiling data and R2 genomics microarray data. A-B, Gene heatmaps were generated using NMF package by comparing GBM tissue data with normal tissue data from TCGA database. C, R2 genomics database were queried for 9 genes comparing normal brain data and GBM tissue data. GBM patient cohorts used are N Brain 172 (Berchtold), N Brain 44 (Harris) as normal control groups and T Glioblastoma 46 (Pfister), T Glioblastoma 70 (Loeffler), T Glioblastoma 84 (Hegi) as GBM tumor groups.

FIG. 9A-C. Globin reduction increases sequencing sensitivity. A, Bar plots showing the number of mRNA reads for samples before and after GR. The red segments represent reads from globin genes. B, Bar plots showing the number of differentially expressed genes surviving FDR≤0.05 before and after GR. The red segments represent up-regulated genes, while blue represent down-regulated. C, Venn diagram showing the overlap of differentially expressed genes surviving FDR≤0.05 before and after GR. d, Sample level scatterplots comparing log 2 normalized counts before and after GR, with post-GR samples plotted along the x-axis and pre-GR samples along the y-axis. The red lines have slope=1 and pass through the origin, and the red dots represent globin genes. Pearson's correlation for each scatterplot is shown.

FIG. 10A-C. Gene, miRNA expression profiling and integrated functional network analysis. A, Dot plot shows Functional Annotation of overlapping Reactome signaling pathways in both GBM blood RNA-seq result and GBM tissue datasets. The x-axis is −log 10 q-value for each pathway. Red dotted line represents the −log 10 transformed q-values (FDR) of 0.1. B, Network connecting DEGs and major overlapped pathways showed in A. C, DEG enrichment analysis using Baylor Module Method. The proportions of DEGs in modules are indicated by a color gradient ranging from blue (100% of transcript decreased) to red (100% of transcripts increased).

FIG. 11A-B. Gene expression difference enrichment analysis. A, Heatmap of 90 narrow-downed DEGs in GBM blood samples from comprehensive analyses. B, Heatmap of log 2 normalized counts for miRNAs that survived P≤0.05 and |log 2FC|≥1.0. For each miRNA, the mean of the control group was subtracted. Hierarchical clustering was performed for the heat maps.

FIG. 12A-D. RT-qPCR validation of selected genes and miRNAs in newly collected blood samples and tissue samples. A, Mean values of multiple PCR results from blood (upper panel) and tissue (lower panel) sample were shown in boxplots. Student t-test, *p<0.05, **p<0.01, ***p<0.001, comparisons: GBM vs. Control (upper panel) or tumor vs. normal (lower panel). B, Heatmap was generated using the means of the PCR results for blood samples with detailed patient clinical information. C, Sensitivities, specificities and area under curve (AUC) were analyzed in current investigated independent samples using ROCit package in R. D, Expressions of the 10 genes in blood and tumor tissue of a GBM patient.

FIG. 13. Percentage of globin gene reads in total mRNA reads per sample.

FIG. 14A-C. Expression comparison of selected genes using TCGA GBM transcriptome profiling data and R2 genomics RNA profiling data. A, Expression of 90 genes in TCGA-GBM tissue data. B, Expression of seven genes (in 10 gene marker candidates) in TCGA-GBM tissue data. C, R2 genomics database were queried for eight genes (in 10 gene marker candidates) comparing normal brain data and GBM tissue data. GBM patient cohorts used are N Brain 172 (Berchtold), N Brain 44 (Harris) as normal control groups and T Glioblastoma 46 (Pfister), T Glioblastoma 70 (Loeffler), T Glioblastoma 84 (Hegi) as GBM tumor groups.

FIG. 15. Analysis of the specificity and sensitivity of the 10 genes. Black dot means Youden index optimal value. The curves were generated with ROCit and ggplot2 packages in R.

### DETAILED DESCRIPTION OF THE INVENTION

Despite advanced marker discovery techniques using liquid biopsies, such as blood and cerebrospinal fluid, for the detection of circulating tumor cells (CTCs), cell-free nucleic acids and extracellular vesicles, there are still few effective GBM specific liquid biomarkers, especially blood-derived markers, available in health care settings to reduce the necessity of multiple invasive brain biopsies and to assist the application of imaging diagnosis and surveillance.

This study aims to develop an innovative integrated approach named as WBGR-Dx (whole blood globin reduction-diagnosis) to accurately detect the differential gene expression using minimally invasive liquid biopsy such as patient whole blood specimens), and to identify a glioblastoma (GBM) blood biomarker/biomarker signature from the discovery phase. Globin reduction markedly increases the sensitivity of differential gene expression detection using RNA-sequencing in human whole blood samples and allows the identification of a pool of differentially expressed genes, including mRNAs, long noncoding RNAs and microRNAs associated with GBM.

By using the combined approach of globin depletion and transcriptome RNA sequencing, the inventors enriched 10 genes (GBM Dx Panel, GDP), the expression configuration of which is likely a common trait of GBM blood and has the potential to be developed as a robust and minimum invasive diagnostic biomarker signature for GBM.

### I. ROC Analysis

In statistics, a receiver operating characteristic (ROC), or ROC curve, is a graphical plot that illustrates the performance of a binary classifier system as its discrimination threshold is varied. ROC analysis may be applied to determine a cut-off value or threshold setting of biomarker expression. For example, patients with biological samples determined to have biomarker expression value above a certain cut-off threshold but below a higher cut-off threshold may be determined to have endometriosis. Patients with biological samples determined to have a biomarker expression level that surpasses the cut-off threshold may be determined to have a disease or condition such as a neurological disorder. The curve is created by plotting the true positive rate against the false positive rate at various threshold settings. (The true-positive rate is also known as sensitivity in biomedical informatics, or recall in machine learning. The false-positive rate is also known as the fall-out and can be calculated as 1−specificity). The ROC curve is thus the sensitivity as a function of fall-out. In general, if the probability distributions for both detection and false alarm are known, the ROC curve can be generated by plotting the cumulative distribution function (area under the probability distribution from −infinity to +infinity) of the detection probability in the y-axis versus the cumulative distribution function of the false-alarm probability in x-axis.

ROC analysis provides tools to select possibly optimal models and to discard suboptimal ones independently from (and prior to specifying) the cost context or the class distribution. ROC analysis is related in a direct and natural way to cost/benefit analysis of diagnostic decision making.

The ROC curve was first developed by electrical engineers and radar engineers during World War II for detecting enemy objects in battlefields and was soon introduced to psychology to account for perceptual detection of stimuli. ROC analysis since then has been used in medicine, radiology, biometrics, and other areas for many decades and is increasingly used in machine learning and data mining research.

The ROC is also known as a relative operating characteristic curve, because it is a comparison of two operating characteristics (TPR and FPR) as the criterion changes. ROC analysis curves are known in the art and described in Metz C E (1978) Basic principles of ROC analysis. Seminars in Nuclear Medicine 8:283-298; Youden W J (1950) An index for rating diagnostic tests. Cancer 3:32-35; Zweig M H, Campbell G (1993) Receiver-operating characteristic (ROC) plots: a fundamental evaluation tool in clinical medicine. Clinical Chemistry 39:561-577; and Greiner M, Pfeiffer D, Smith R D (2000) Principles and practical application of the receiver-operating characteristic analysis for diagnostic tests. Preventive Veterinary Medicine 45:23-41, which are herein incorporated by reference in their entirety. A ROC analysis may be used to create cut-off values for prognosis and/or diagnosis purposes.

### II. Protein Assays

A variety of techniques can be employed to measure expression levels of polypeptides and proteins in a biological sample to determine biomarker expression levels. Examples of such formats include, but are not limited to, enzyme immunoassay (EIA), radioimmunoassay (RIA), Western blot analysis and enzyme linked immunoabsorbant assay (ELISA). A skilled artisan can readily adapt known protein/antibody detection methods for use in determining protein expression levels of biomarkers.

In one embodiment, antibodies, or antibody fragments or derivatives, can be used in methods such as Western blots, ELISA, or immunofluorescence techniques to detect biomarker expression. In some embodiments, either the antibodies or proteins are immobilized on a solid support. Suitable solid phase supports or carriers include any support capable of binding an antigen or an antibody. Well-known supports or carriers include glass, polystyrene, polypropylene, polyethylene, dextran, nylon, amylases, natural and modified celluloses, polyacrylamides, gabbros, and magnetite.

One skilled in the art will know many other suitable carriers for binding antibody or antigen, and will be able to adapt such support for use with the present disclosure. The support can then be washed with suitable buffers followed by treatment with the detectably labeled antibody. The solid phase support can then be washed with the buffer a second time to remove unbound antibody. The amount of bound label on the solid support can then be detected by conventional means.

Immunohistochemistry methods are also suitable for detecting the expression levels of biomarkers. In some embodiments, antibodies or antisera, including polyclonal antisera, and monoclonal antibodies specific for each marker may be used to detect expression. The antibodies can be detected by direct labeling of the antibodies themselves, for example, with radioactive labels, fluorescent labels, hapten labels such as, biotin, or an enzyme such as horseradish peroxidase or alkaline phosphatase. Alternatively, unlabeled primary antibody is used in conjunction with a labeled secondary antibody, comprising antisera, polyclonal antisera or a monoclonal antibody specific for the primary antibody. Immunohistochemistry protocols and kits are well known in the art and are commercially available.

Immunological methods for detecting and measuring complex formation as a measure of protein expression using either specific polyclonal or monoclonal antibodies are known in the art. Examples of such techniques include enzyme-linked immunosorbent assays (ELISAs), radioimmunoassays (RIAs), fluorescence-activated cell sorting (FACS) and antibody arrays. Such immunoassays typically involve the measurement of complex formation between the protein and its specific antibody. These assays and their quantitation against purified, labeled standards are well known in the art. A two-site, monoclonal-based immunoassay utilizing antibodies reactive to two non-interfering epitopes or a competitive binding assay may be employed.

Numerous labels are available and commonly known in the art. Radioisotope labels include, for example, 36S, 14C, 125I, 3H, and 131I. The antibody can be labeled with the radioisotope using the techniques known in the art. Fluorescent labels include, for example, labels such as rare earth chelates (europium chelates) or fluorescein and its derivatives, rhodamine and its derivatives, dansyl, Lissamine, phycoerythrin and Texas Red are available. The fluorescent labels can be conjugated to the antibody variant using the techniques known in the art. Fluorescence can be quantified using a fluorimeter. Various enzyme-substrate labels are available and U.S. Pat. Nos. 4,275,149, 4,318,980 provides a review of some of these. The enzyme generally catalyzes a chemical alteration of the chromogenic substrate which can be measured using various techniques. For example, the enzyme may catalyze a color change in a substrate, which can be measured spectrophotometrically. Alternatively, the enzyme may alter the fluorescence or chemiluminescence of the substrate. Techniques for quantifying a change in fluorescence are described above. The chemiluminescent substrate becomes electronically excited by a chemical reaction and may then emit light which can be measured (using a chemiluminometer, for example) or donates energy to a fluorescent acceptor. Examples of enzymatic labels include luciferases (e.g., firefly luciferase and bacterial luciferase; U.S. Pat. No. 4,737,456), luciferin, 2,3-dihydrophthalazinediones, malate dehydrogenase, urease, peroxidase such as horseradish peroxidase (HRPO), alkaline phosphatase, .beta.-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like. Techniques for conjugating enzymes to antibodies are described in O'Sullivan et al., Methods for the Preparation of Enzyme-Antibody Conjugates for Use in Enzyme Immunoassay, in Methods in Enzymology (Ed. J. Langone & H. Van Vunakis), Academic press, New York, 73: 147-166 (1981).

In some embodiments, a detection label is indirectly conjugated with an antibody. The skilled artisan will be aware of various techniques for achieving this. For example, the antibody can be conjugated with biotin and any of the three broad categories of labels mentioned above can be conjugated with avidin, or vice versa. Biotin binds selectively to avidin and thus, the label can be conjugated with the antibody in this indirect manner. Alternatively, to achieve indirect conjugation of the label with the antibody, the antibody is conjugated with a small hapten (e.g., digoxin) and one of the different types of labels mentioned above is conjugated with an anti-hapten antibody (e.g., anti-digoxin antibody). In some embodiments, the antibody need not be labeled, and the presence thereof can be detected using a labeled antibody, which binds to the antibody.

### III. Nucleic Acid Assays

Aspects of the methods include assaying nucleic acids to determine expression or activity levels. Arrays can be used to detect differences between two samples. Specifically contemplated applications include identifying and/or quantifying differences between RNA from a sample that is normal and from a sample that is not normal, between a cancerous condition and a non-cancerous condition, between one cancerous condition, such as fast doubling time cells and another cancer condition, such as slow doubling time cells, or between two differently treated samples. Also, RNA may be compared between a sample believed to be susceptible to a particular disease or condition and one believed to be not susceptible or resistant to that disease or condition. A sample that is not normal is one exhibiting phenotypic trait(s) of a disease or condition or one believed to be not normal with respect to that disease or condition. It may be compared to a cell that is normal with respect to that disease or condition. Phenotypic traits include symptoms of, or susceptibility to, a disease or condition of which a component is or may or may not be genetic or caused by a hyperproliferative or neoplastic cell or cells.

To determine expression levels of a biomarker, an array may be used. An array comprises a solid support with nucleic acid probes attached to the support. Arrays typically comprise a plurality of different nucleic acid probes that are coupled to a surface of a substrate in different, known locations. These arrays, also described as “microarrays” or colloquially “chips” have been generally described in the art, for example, U.S. Pat. Nos. 5,143,854, 5,445,934, 5,744,305, 5,677,195, 6,040,193, 5,424,186 and Fodor et al., 1991), each of which is incorporated by reference in its entirety for all purposes. Techniques for the synthesis of these arrays using mechanical synthesis methods are described in, e.g., U.S. Pat. No. 5,384,261, incorporated herein by reference in its entirety for all purposes. Although a planar array surface is used in certain aspects, the array may be fabricated on a surface of virtually any shape or even a multiplicity of surfaces. Arrays may be nucleic acids on beads, gels, polymeric surfaces, fibers such as fiber optics, glass or any other appropriate substrate, see U.S. Pat. Nos. 5,770,358, 5,789,162, 5,708,153, 6,040,193 and 5,800,992, which are hereby incorporated in their entirety for all purposes.

Further assays useful for determining biomarker expression include, but are not limited to, nucleic amplification, polymerase chain reaction, quantitative PCR, RT-PCR, in situ hybridization, Northern hybridization, hybridization protection assay (HPA) (GenProbe), branched DNA (bDNA) assay (Chiron), rolling circle amplification (RCA), single molecule hybridization detection (US Genomics), Invader assay (ThirdWave Technologies), and/or Bridge Litigation Assay (Genaco).

A further assay useful for quantifying and/or identifying nucleic acids, such as nucleic acids comprising biomarker genes, is RNA-seq. RNA-seq (RNA sequencing), also called whole transcriptome shotgun sequencing, uses next-generation sequencing (NGS) to reveal the presence and quantity of RNA in a biological sample at a given moment in time. RNA-Seq is used to analyze the continually changing cellular transcriptome. Specifically, RNA-Seq facilitates the ability to look at alternative gene spliced transcripts, post-transcriptional modifications, gene fusion, mutations/SNPs and changes in gene expression. In addition to mRNA transcripts, RNASeq can look at different populations of RNA to include total RNA, small RNA, such as miRNA, tRNA, and ribosomal profiling. RNA-Seq can also be used to determine exon/intron boundaries and verify or amend previously annotated 5′ and 3′ gene boundaries.

To normalize the expression values of one gene among different samples, comparing the RNA and/or miRNA level of interest in the samples from the subject object of study with a control RNA level is possible. As it is used herein, a “control RNA” is an RNA of a gene for which the expression level does not differ among different non-diseased individuals. In some aspects, the gene may be constitutively expressed in all types of cells. A control RNA is preferably an mRNA derived from a housekeeping gene encoding a protein that is constitutively expressed and carrying out essential cell functions. A known amount of a control RNA may be added to the sample(s) and the value measured for the level of the RNA of interest may be normalized to the value measured for the known amount of the control RNA. Normalization for some methods, such as for sequencing, may comprise calculating the reads per kilobase of transcript per million mapped reads (RPKM) for a gene of interest, or may comprise calculating the fragments per kilobase of transcript per million mapped reads (FPKM) for a gene of interest. Normalization methods may comprise calculating the log 2-transformed count per million (log-CPM). It can be appreciated to one skilled in the art that any method of normalization that accurately calculates the expression value of an RNA for comparison between samples may be used.

Methods disclosed herein may include comparing a measured expression level to a reference expression level. The term “reference expression level” refers to a value used as a reference for the values/data obtained from samples obtained from patients. The reference level can be an absolute value, a relative value, a value which has an upper and/or lower limit, a series of values, an average value, a median, a mean value, or a value expressed by reference to a control or reference value. A reference level can be based on the value obtained from an individual sample, such as, for example, a value obtained from a sample from the subject object of study but obtained at a previous point in time. The reference level can be based on a high number of samples, such as the levels obtained in a cohort of subjects having a particular characteristic. The reference level may be defined as the mean level of the patients in the cohort. The reference may be from subjects that are healthy, subjects without one or more neurological disorder(s), subjects that are age-matched, subjects that are gender-matched, and/or subjects that are race-matched. A reference level can be based on the expression levels of the markers to be compared obtained from samples from subjects who do not have a disease state or a particular phenotype. The person skilled in the art will see that the particular reference expression level can vary depending on the specific method to be performed.

Some embodiments include determining that a measured expression level is higher than, lower than, increased relative to, decreased relative to, equal to, or within a predetermined amount of a reference expression level. In some embodiments, a higher, lower, increased, or decreased expression level is at least 1, 1.5, 2, 2.5, 3, 3.5, 4, 4.5, 5, 5.5, 6, 7, 8, 9, 10, 12, 14, 16, 18, 20, 50, 100, 150, 200, 250, 500, or 1000 fold (or any derivable range therein) or at least 10, 20, 30, 40, 50, 60, 70, 80, 90, 100, 200, 300, 400, 500, 600, 700, 800, or 900% different than the reference level, or any derivable range therein. These values may represent a predetermined threshold level, and some embodiments include determining that the measured expression level is higher by a predetermined amount or lower by a predetermined amount than a reference level. In some embodiments, a level of expression may be qualified as “low” or “high,” which indicates the patient expresses a certain gene or RNA at a level relative to a reference level or a level with a range of reference levels that are determined from multiple samples meeting particular criteria. The level or range of levels in multiple control samples is an example of this. In some embodiments, that certain level or a predetermined threshold value is at, below, or above 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percentile, or any range derivable therein. Moreover, a threshold level may be derived from a cohort of individuals meeting a particular criterion or set of criteria. The number in the cohort may be, be at least, or be at most 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600,700, 800, 900, 1000, 1100, 1200, 1300, 1400, 1500, 1600, 1700, 1800, 1900, 2000 or more (or any range derivable therein). A measured expression level can be considered equal to a reference expression level if it is within a certain amount of the reference expression level, and such amount may be an amount that is predetermined. The predetermined amount may be within 0.1, 0.2, 0.3, 0.4, 0.5, 0.6, 0.7, 0.8, 0.9, 1.0, 2, 3, 4, 5, 6, 7, 8, 9, 10, 11, 12, 13, 14, 15, 16, 17, 18, 19, 20, 21, 22, 23, 24, 25, 30, 35, 40, 45, or 50% of the reference level, or any range derivable therein.

For any comparison of gene and/or RNA expression levels to a mean expression level or a reference expression level, the comparison is to be made on a gene-by-gene and RNA-by-RNA basis.

### IV. Sample Preparation

In certain aspects, methods involve obtaining a sample from a subject. The methods of obtaining provided herein may include methods of biopsy such as fine needle aspiration, core needle biopsy, vacuum assisted biopsy, incisional biopsy, excisional biopsy, punch biopsy, shave biopsy or skin biopsy. In certain embodiments the sample is obtained from a biopsy from ovarian or endometrial tissue by any of the biopsy methods previously mentioned. In other embodiments the sample may be obtained from any of the tissues provided herein that include but are not limited to non-cancerous or cancerous tissue and non-cancerous or cancerous tissue from the ovarian epithelium, fallopian epithelium, ovaries, cervix, fallopian tube, or uterus. Alternatively, the sample may be obtained from any other source including but not limited to blood, serum, plasma, sweat, hair follicle, buccal tissue, tears, menses, feces, or saliva. In certain aspects of the current methods, any medical professional such as a doctor, nurse or medical technician may obtain a biological sample for testing. Yet further, the biological sample can be obtained without the assistance of a medical professional.

A sample may include but is not limited to, tissue, cells, or biological material from cells or derived from cells of a subject. The biological sample may be a heterogeneous or homogeneous population of cells or tissues. The biological sample may be obtained using any method known to the art that can provide a sample suitable for the analytical methods described herein. The sample may be obtained by non-invasive methods including but not limited to: scraping of the skin or cervix, swabbing of the cheek, saliva collection, urine collection, feces collection, collection of menses, tears, or semen.

The sample may be obtained by methods known in the art. In certain embodiments the samples are obtained by biopsy. In other embodiments the sample is obtained by swabbing, endoscopy, scraping, phlebotomy, or any other methods known in the art. In some cases, the sample may be obtained, stored, or transported using components of a kit of the present methods. In some cases, multiple samples, such as multiple plasma or serum samples may be obtained for diagnosis by the methods described herein. In other cases, multiple samples, such as one or more samples from one tissue type (for example ovaries or related tissues) and one or more samples from another specimen (for example serum) may be obtained for diagnosis by the methods. Samples may be obtained at different times are stored and/or analyzed by different methods. For example, a sample may be obtained and analyzed by routine staining methods or any other cytological analysis methods.

In some embodiments the biological sample may be obtained by a physician, nurse, or other medical professional such as a medical technician, endocrinologist, cytologist, phlebotomist, radiologist, or a pulmonologist. The medical professional may indicate the appropriate test or assay to perform on the sample. In certain aspects a molecular profiling business may consult on which assays or tests are most appropriately indicated. In further aspects of the current methods, the patient or subject may obtain a biological sample for testing without the assistance of a medical professional, such as obtaining a whole blood sample, a urine sample, a fecal sample, a buccal sample, or a saliva sample.

In other cases, the sample is obtained by an invasive procedure including but not limited to: biopsy, needle aspiration, blood draw, endoscopy, or phlebotomy. The method of needle aspiration may further include fine needle aspiration, core needle biopsy, vacuum assisted biopsy, or large core biopsy. In some embodiments, multiple samples may be obtained by the methods herein to ensure a sufficient amount of biological material.

General methods for obtaining biological samples are also known in the art. Publications such as Ramzy, Ibrahim Clinical Cytopathology and Aspiration Biopsy 2001, which is herein incorporated by reference in its entirety, describes general methods for biopsy and cytological methods.

In some embodiments of the present methods, the molecular profiling business may obtain the biological sample from a subject directly, from a medical professional, from a third party, or from a kit provided by a molecular profiling business or a third party. In some cases, the biological sample may be obtained by the molecular profiling business after the subject, a medical professional, or a third party acquires and sends the biological sample to the molecular profiling business. In some cases, the molecular profiling business may provide suitable containers, and excipients for storage and transport of the biological sample to the molecular profiling business.

In some embodiments of the methods described herein, a medical professional need not be involved in the initial diagnosis or sample acquisition. An individual may alternatively obtain a sample through the use of an over the counter (OTC) kit. An OTC kit may contain a means for obtaining said sample as described herein, a means for storing said sample for inspection, and instructions for proper use of the kit. In some cases, molecular profiling services are included in the price for purchase of the kit. In other cases, the molecular profiling services are billed separately. A sample suitable for use by the molecular profiling business may be any material containing tissues, cells, nucleic acids, genes, gene fragments, expression products, gene expression products, or gene expression product fragments of an individual to be tested. Methods for determining sample suitability and/or adequacy are provided.

In some embodiments, the subject may be referred to a specialist such as an oncologist, surgeon, or endocrinologist. The specialist may likewise obtain a biological sample for testing or refer the individual to a testing center or laboratory for submission of the biological sample. In some cases the medical professional may refer the subject to a testing center or laboratory for submission of the biological sample. In other cases, the subject may provide the sample. In some cases, a molecular profiling business may obtain the sample.

### V. Administration of Therapeutic Compositions

The therapy provided herein may comprise administration of a combination of therapeutic agents, such as a first cancer therapy and a second cancer therapy. The therapies may be administered in any suitable manner known in the art. For example, the first and second cancer treatment may be administered sequentially (at different times) or concurrently (at the same time). In some embodiments, the first and second cancer treatments are administered in a separate composition. In some embodiments, the first and second cancer treatments are in the same composition.

Embodiments of the disclosure relate to compositions and methods comprising therapeutic compositions. The different therapies may be administered in one composition or in more than one composition, such as 2 compositions, 3 compositions, or 4 compositions. Various combinations of the agents may be employed.

The therapeutic agents of the disclosure may be administered by the same route of administration or by different routes of administration. In some embodiments, the cancer therapy is administered intravenously, intramuscularly, subcutaneously, topically, orally, transdermally, intraperitoneally, intraorbitally, by implantation, by inhalation, intrathecally, intraventricularly, or intranasally. In some embodiments, the antibiotic is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

### VI. Treatment Embodiments for GBM

### A. Immunostimulators

In some embodiments, the method further comprises administration of a treatment. In some embodiments, the treatment is an immunostimulator. The term “immunostimulator” as used herein refers to a compound that can stimulate an immune response in a subject, and may include an adjuvant. In some embodiments, an immunostimulator is an agent that does not constitute a specific antigen, but can boost the strength and longevity of an immune response to an antigen. Such immunostimulators may include, but are not limited to stimulators of pattern recognition receptors, such as Toll-like receptors, RIG-1 and NOD-like receptors (NLR), mineral salts, such as alum, alum combined with monphosphoryl lipid (MPL) A of Enterobacteria, such as Escherichia coli, Salmonella minnesota, Salmonella typhimurium, or Shigella flexneri or specifically with MPL (AS04), MPL A of above-mentioned bacteria separately, saponins, such as QS-21, Quil-A, ISCOMs, ISCOMATRIX, emulsions such as MF59, Montanide, ISA 51 and ISA 720, AS02 (QS21+squalene+MPL.), liposomes and liposomal formulations such as AS01, synthesized or specifically prepared microparticles and microcarriers such as bacteria-derived outer membrane vesicles (OMV) of N. gonorrheae, Chlamydia trachomatis and others, or chitosan particles, depot-forming agents, such as Pluronic block co-polymers, specifically modified or prepared peptides, such as muramyl dipeptide, aminoalkyl glucosaminide 4-phosphates, such as RC529, or proteins, such as bacterial toxoids or toxin fragments.

In some embodiments, the treatment comprises an agonist for pattern recognition receptors (PRR), including, but not limited to Toll-Like Receptors (TLRs), specifically TLRs 2, 3, 4, 5, 7, 8, 9 and/or combinations thereof. In some embodiments, treatments comprise agonists for Toll-Like Receptors 3, agonists for Toll-Like Receptors 7 and 8, or agonists for Toll-Like Receptor 9; preferably the recited immunostimulators comprise imidazoquinolines; such as R848; adenine derivatives, such as those disclosed in U.S. Pat. No. 6,329,381, U.S. Published Patent Application 2010/0075995, or WO 2010/018132; immunostimulatory DNA; or immunostimulatory RNA. In some embodiments, the treatment also may comprise immunostimulatory RNA molecules, such as but not limited to dsRNA, poly I:C or poly I:poly C12U (available as Ampligen®, both poly I:C and poly I:polyC12U being known as TLR3 stimulants), and/or those disclosed in F. Heil et al., “Species-Specific Recognition of Single-Stranded RNA via Toll-like Receptor 7 and 8” Science 303(5663), 1526-1529 (2004); J. Vollmer et al., “Immune modulation by chemically modified ribonucleosides and oligoribonucleotides” WO 2008033432 A2; A. Forsbach et al., “Immunostimulatory oligoribonucleotides containing specific sequence motif(s) and targeting the Toll-like receptor 8 pathway” WO 2007062107 A2; E. Uhlmann et al., “Modified oligoribonucleotide analogs with enhanced immunostimulatory activity” U.S. Pat. Appl. Publ. US 2006241076; G. Lipford et al., “Immunostimulatory viral RNA oligonucleotides and use for treating cancer and infections” WO 2005097993 A2; G. Lipford et al., “Immunostimulatory G,U-containing oligoribonucleotides, compositions, and screening methods” WO 2003086280 A2. In some embodiments, a treatment may be a TLR-4 agonist, such as bacterial lipopolysaccharide (LPS), VSV-G, and/or HMGB-1. In some embodiments, treatments may comprise TLR-5 agonists, such as flagellin, or portions or derivatives thereof, including but not limited to those disclosed in U.S. Pat. Nos. 6,130,082, 6,585,980, and 7,192,725.

In some embodiments, treatments may be proinflammatory stimuli released from necrotic cells (e.g., urate crystals). In some embodiments, treatments may be activated components of the complement cascade (e.g., CD21, CD35, etc.). In some embodiments, treatments may be activated components of immune complexes. Treatments also include complement receptor agonists, such as a molecule that binds to CD21 or CD35. In some embodiments, the complement receptor agonist induces endogenous complement opsonization of the synthetic nanocarrier. In some embodiments, immunostimulators are cytokines, which are small proteins or biological factors (in the range of 5 kD-20 kD) that are released by cells and have specific effects on cell-cell interaction, communication and behavior of other cells. In some embodiments, the cytokine receptor agonist is a small molecule, antibody, fusion protein, or aptamer.

### B. Immunotherapies

In some embodiments, the treatment comprises a cancer immunotherapy. Cancer immunotherapy (sometimes called immuno-oncology, abbreviated IO) is the use of the immune system to treat cancer. Immunotherapies can be categorized as active, passive or hybrid (active and passive). These approaches exploit the fact that cancer cells often have molecules on their surface that can be detected by the immune system, known as tumour-associated antigens (TAAs); they are often proteins or other macromolecules (e.g. carbohydrates). Active immunotherapy directs the immune system to attack tumor cells by targeting TAAs. Passive immunotherapies enhance existing anti-tumor responses and include the use of monoclonal antibodies, lymphocytes and cytokines. Immumotherapies are known in the art, and some are described below.

### Inhibition of Co-Stimulatory Molecules

In some embodiments, the immunotherapy comprises an inhibitor of a co-stimulatory molecule. In some embodiments, the inhibitor comprises an inhibitor of B7-1 (CD80), B7-2 (CD86), CD28, ICOS, OX40 (TNFRSF4), 4-1BB (CD137; TNFRSF9), CD40L (CD40LG), GITR (TNFRSF18), and combinations thereof. Inhibitors include inhibitory antibodies, polypeptides, compounds, and nucleic acids.

### C. Dendritic Cell Therapy

Dendritic cell therapy provokes anti-tumor responses by causing dendritic cells to present tumor antigens to lymphocytes, which activates them, priming them to kill other cells that present the antigen. Dendritic cells are antigen presenting cells (APCs) in the mammalian immune system. In cancer treatment they aid cancer antigen targeting. One example of cellular cancer therapy based on dendritic cells is sipuleucel-T.

One method of inducing dendritic cells to present tumor antigens is by vaccination with autologous tumor lysates or short peptides (small parts of protein that correspond to the protein antigens on cancer cells). These peptides are often given in combination with adjuvants (highly immunogenic substances) to increase the immune and anti-tumor responses. Other adjuvants include proteins or other chemicals that attract and/or activate dendritic cells, such as granulocyte macrophage colony-stimulating factor (GM-CSF).

Dendritic cells can also be activated in vivo by making tumor cells express GM-CSF. This can be achieved by either genetically engineering tumor cells to produce GM-CSF or by infecting tumor cells with an oncolytic virus that expresses GM-CSF.

Another strategy is to remove dendritic cells from the blood of a patient and activate them outside the body. The dendritic cells are activated in the presence of tumor antigens, which may be a single tumor-specific peptide/protein or a tumor cell lysate (a solution of broken down tumor cells). These cells (with optional adjuvants) are infused and provoke an immune response.

Dendritic cell therapies include the use of antibodies that bind to receptors on the surface of dendritic cells. Antigens can be added to the antibody and can induce the dendritic cells to mature and provide immunity to the tumor. Dendritic cell receptors such as TLR3, TLR7, TLR8 or CD40 have been used as antibody targets.

### D. CAR-T Cell Therapy

Chimeric antigen receptors (CARs, also known as chimeric immunoreceptors, chimeric T cell receptors or artificial T cell receptors) are engineered receptors that combine a new specificity with an immune cell to target cancer cells. Typically, these receptors graft the specificity of a monoclonal antibody onto a T cell. The receptors are called chimeric because they are fused of parts from different sources. CAR-T cell therapy refers to a treatment that uses such transformed cells for cancer therapy.

The basic principle of CAR-T cell design involves recombinant receptors that combine antigen-binding and T-cell activating functions. The general premise of CAR-T cells is to artificially generate T-cells targeted to markers found on cancer cells. Scientists can remove T-cells from a person, genetically alter them, and put them back into the patient for them to attack the cancer cells. Once the T cell has been engineered to become a CAR-T cell, it acts as a “living drug”. CAR-T cells create a link between an extracellular ligand recognition domain to an intracellular signalling molecule which in turn activates T cells. The extracellular ligand recognition domain is usually a single-chain variable fragment (scFv). An important Aspect of the safety of CAR-T cell therapy is how to ensure that only cancerous tumor cells are targeted, and not normal cells. The specificity of CAR-T cells is determined by the choice of molecule that is targeted.

Exemplary CAR-T therapies include Tisagenlecleucel (Kymriah) and Axicabtagene ciloleucel (Yescarta). In some embodiments, the CAR-T therapy targets CD19.

### E. Cytokine Therapy

Cytokines are proteins produced by many types of cells present within a tumor. They can modulate immune responses. The tumor often employs them to allow it to grow and reduce the immune response. These immune-modulating effects allow them to be used as drugs to provoke an immune response. Two commonly used cytokines are interferons and interleukins.

Interferons are produced by the immune system. They are usually involved in anti-viral response, but also have use for cancer. They fall in three groups: type I (IFNα and IFNβ), type II (IFNγ) and type III (IFNλ).

Interleukins have an array of immune system effects. IL-2 is an exemplary interleukin cytokine therapy.

### 1. Adoptive T-Cell Therapy

Adoptive T cell therapy is a form of passive immunization by the transfusion of T-cells (adoptive cell transfer). They are found in blood and tissue and usually activate when they find foreign pathogens. Specifically they activate when the T-cell's surface receptors encounter cells that display parts of foreign proteins on their surface antigens. These can be either infected cells, or antigen presenting cells (APCs). They are found in normal tissue and in tumor tissue, where they are known as tumor infiltrating lymphocytes (TILs). They are activated by the presence of APCs such as dendritic cells that present tumor antigens. Although these cells can attack the tumor, the environment within the tumor is highly immunosuppressive, preventing immune-mediated tumour death.

Multiple ways of producing and obtaining tumour targeted T-cells have been developed. T-cells specific to a tumor antigen can be removed from a tumor sample (TILs) or filtered from blood. Subsequent activation and culturing is performed ex vivo, with the results reinfused. Activation can take place through gene therapy, or by exposing the T cells to tumor antigens.

### 2. Checkpoint Inhibitors and Combination Treatment

In some embodiments, the treatment comprises immune checkpoint inhibitors. Certain embodiments are further described below.

a. PD-1, PDL1, and PDL2 Inhibitors

PD-1 can act in the tumor microenvironment where T cells encounter an infection or tumor. Activated T cells upregulate PD-1 and continue to express it in the peripheral tissues. Cytokines such as IFN-gamma induce the expression of PDL1 on epithelial cells and tumor cells. PDL2 is expressed on macrophages and dendritic cells. The main role of PD-1 is to limit the activity of effector T cells in the periphery and prevent excessive damage to the tissues during an immune response. Inhibitors of the disclosure may block one or more functions of PD-1 and/or PDL1 activity.

Alternative names for “PD-1” include CD279 and SLEB2. Alternative names for “PDL1” include B7-H1, B7-4, CD274, and B7-H. Alternative names for “PDL2” include B7-DC, Btdc, and CD273. In some embodiments, PD-1, PDL1, and PDL2 are human PD-1, PDL1 and PDL2.

In some embodiments, the PD-1 inhibitor is a molecule that inhibits the binding of PD-1 to its ligand binding partners. In a specific aspect, the PD-1 ligand binding partners are PDL1 and/or PDL2. In another embodiment, a PDL1 inhibitor is a molecule that inhibits the binding of PDL1 to its binding partners. In a specific aspect, PDL1 binding partners are PD-1 and/or B7-1. In another embodiment, the PDL2 inhibitor is a molecule that inhibits the binding of PDL2 to its binding partners. In a specific aspect, a PDL2 binding partner is PD-1. The inhibitor may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 inhibitors for use in the methods and compositions provided herein are known in the art such as described in U.S. Patent Application Nos. US2014/0294898, US2014/022021, and US2011/0008369, all incorporated herein by reference.

In some embodiments, the PD-1 inhibitor is an anti-PD-1 antibody (e.g., a human antibody, a humanized antibody, or a chimeric antibody). In some embodiments, the anti-PD-1 antibody is selected from the group consisting of nivolumab, pembrolizumab, and pidilizumab. In some embodiments, the PD-1 inhibitor is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PDL1 inhibitor comprises AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. Pidilizumab, also known as CT-011, hBAT, or hBAT-1, is an anti-PD-1 antibody described in WO2009/101611. AMP-224, also known as B7-DCIg, is a PDL2-Fc fusion soluble receptor described in WO2010/027827 and WO2011/066342. Additional PD-1 inhibitors include MEDI0680, also known as AMP-514, and REGN2810.

In some embodiments, the immune checkpoint inhibitor is a PDL1 inhibitor such as Durvalumab, also known as MEDI4736, atezolizumab, also known as MPDL3280A, avelumab, also known as MSB00010118C, MDX-1105, BMS-936559, or combinations thereof. In certain aspects, the immune checkpoint inhibitor is a PDL2 inhibitor such as rHIgM12B7.

In some embodiments, the inhibitor comprises the heavy and light chain CDRs or VRs of nivolumab, pembrolizumab, or pidilizumab. Accordingly, in one embodiment, the inhibitor comprises the CDR1, CDR2, and CDR3 domains of the VH region of nivolumab, pembrolizumab, or pidilizumab, and the CDR1, CDR2 and CDR3 domains of the VL region of nivolumab, pembrolizumab, or pidilizumab. In another embodiment, the antibody competes for binding with and/or binds to the same epitope on PD-1, PDL1, or PDL2 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

b. CTLA-4, B7-1, and B7-2

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to B7-1 (CD80) or B7-2 (CD86) on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to B7-1 and B7-2 on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules. Inhibitors of the disclosure may block one or more functions of CTLA-4, B7-1, and/or B7-2 activity. In some embodiments, the inhibitor blocks the CTLA-4 and B7-1 interaction. In some embodiments, the inhibitor blocks the CTLA-4 and B7-2 interaction.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al., 1998; can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

A further anti-CTLA-4 antibody useful as a checkpoint inhibitor in the methods and compositions of the disclosure is ipilimumab (also known as 10D1, MDX-010, MDX-101, and Yervoy®) or antigen binding fragments and variants thereof (see, e.g., WO01/14424).

In some embodiments, the inhibitor comprises the heavy and light chain CDRs or VRs of tremelimumab or ipilimumab. Accordingly, in one embodiment, the inhibitor comprises the CDR1, CDR2, and CDR3 domains of the VH region of tremelimumab or ipilimumab, and the CDR1, CDR2 and CDR3 domains of the VL region of tremelimumab or ipilimumab. In another embodiment, the antibody competes for binding with and/or binds to the same epitope on PD-1, B7-1, or B7-2 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

### F. Oncolytic Virus

In some embodiments, the treatment comprises an oncolytic virus. An oncolytic virus is a virus that preferentially infects and kills cancer cells. As the infected cancer cells are destroyed by oncolysis, they release new infectious virus particles or virions to help destroy the remaining tumour. Oncolytic viruses are thought not only to cause direct destruction of the tumour cells, but also to stimulate host anti-tumour immune responses for long-term immunotherapy

### G. Polysaccharides

In some embodiments, the treatment comprises polysaccharides. Certain compounds found in mushrooms, primarily polysaccharides, can up-regulate the immune system and may have anti-cancer properties. For example, beta-glucans such as lentinan have been shown in laboratory studies to stimulate macrophage, NK cells, T cells and immune system cytokines and have been investigated in clinical trials as immunologic adjuvants.

### H. Neoantigens

In some embodiments, the treatment comprises neoantigen administration. Many tumors express mutations. These mutations potentially create new targetable antigens (neoantigens) for use in T cell immunotherapy. The presence of CD8+ T cells in cancer lesions, as identified using RNA sequencing data, is higher in tumors with a high mutational burden. The level of transcripts associated with cytolytic activity of natural killer cells and T cells positively correlates with mutational load in many human tumors.

### I. Chemotherapies

In some embodiments, the treatment comprises a chemotherapy. Suitable classes of chemotherapeutic agents include (a) Alkylating Agents, such as nitrogen mustards (e.g., mechlorethamine, cylophosphamide, ifosfamide, melphalan, chlorambucil), ethylenimines and methylmelamines (e.g., hexamethylmelamine, thiotepa), alkyl sulfonates (e.g., busulfan), nitrosoureas (e.g., carmustine, lomustine, chlorozoticin, streptozocin) and triazines (e.g., dicarbazine), (b) Antimetabolites, such as folic acid analogs (e.g., methotrexate), pyrimidine analogs (e.g., 5-fluorouracil, floxuridine, cytarabine, azauridine) and purine analogs and related materials (e.g., 6-mercaptopurine, 6-thioguanine, pentostatin), (c) Natural Products, such as vinca alkaloids (e.g., vinblastine, vincristine), epipodophylotoxins (e.g., etoposide, teniposide), antibiotics (e.g., dactinomycin, daunorubicin, doxorubicin, bleomycin, plicamycin and mitoxanthrone), enzymes (e.g., L-asparaginase), and biological response modifiers (e.g., Interferon-α), and (d) Miscellaneous Agents, such as platinum coordination complexes (e.g., cisplatin, carboplatin), substituted ureas (e.g., hydroxyurea), methylhydiazine derivatives (e.g., procarbazine), and adreocortical suppressants (e.g., taxol and mitotane). In some embodiments, cisplatin is a particularly suitable chemotherapeutic agent.

Cisplatin has been widely used to treat cancers such as, for example, metastatic testicular or ovarian carcinoma, advanced bladder cancer, head or neck cancer, cervical cancer, lung cancer or other tumors. Cisplatin is not absorbed orally and must therefore be delivered via other routes such as, for example, intravenous, subcutaneous, intratumoral or intraperitoneal injection. Cisplatin can be used alone or in combination with other agents, with efficacious doses used in clinical applications including about 15 mg/m2 to about 20 mg/m2 for 5 days every three weeks for a total of three courses being contemplated in certain embodiments. In some embodiments, the amount of cisplatin delivered to the cell and/or subject in conjunction with the construct comprising an Egr-1 promoter operably linked to a polynucleotide encoding the therapeutic polypeptide is less than the amount that would be delivered when using cisplatin alone.

Other suitable chemotherapeutic agents include antimicrotubule agents, e.g., Paclitaxel (“Taxol”) and doxorubicin hydrochloride (“doxorubicin”). The combination of an Egr-1 promoter/TNFα construct delivered via an adenoviral vector and doxorubicin was determined to be effective in overcoming resistance to chemotherapy and/or TNF-α, which suggests that combination treatment with the construct and doxorubicin overcomes resistance to both doxorubicin and TNF-α.

Doxorubicin is absorbed poorly and is preferably administered intravenously. In certain embodiments, appropriate intravenous doses for an adult include about 60 mg/m2 to about 75 mg/m2 at about 21-day intervals or about 25 mg/m2 to about 30 mg/m2 on each of 2 or 3 successive days repeated at about 3 week to about 4 week intervals or about 20 mg/m2 once a week. The lowest dose should be used in elderly patients, when there is prior bone-marrow depression caused by prior chemotherapy or neoplastic marrow invasion, or when the drug is combined with other myelopoietic suppressant drugs.

Nitrogen mustards are another suitable chemotherapeutic agent useful in the methods of the disclosure. A nitrogen mustard may include, but is not limited to, mechlorethamine (HN2), cyclophosphamide and/or ifosfamide, melphalan (L-sarcolysin), and chlorambucil. Cyclophosphamide (CYTOXAN®) is available from Mead Johnson and NEOSTAR® is available from Adria), is another suitable chemotherapeutic agent. Suitable oral doses for adults include, for example, about 1 mg/kg/day to about 5 mg/kg/day, intravenous doses include, for example, initially about 40 mg/kg to about 50 mg/kg in divided doses over a period of about 2 days to about 5 days or about 10 mg/kg to about 15 mg/kg about every 7 days to about 10 days or about 3 mg/kg to about 5 mg/kg twice a week or about 1.5 mg/kg/day to about 3 mg/kg/day. Because of adverse gastrointestinal effects, the intravenous route is preferred. The drug also sometimes is administered intramuscularly, by infiltration or into body cavities.

Additional suitable chemotherapeutic agents include pyrimidine analogs, such as cytarabine (cytosine arabinoside), 5-fluorouracil (fluouracil; 5-FU) and floxuridine (fluorode-oxyuridine; FudR). 5-FU may be administered to a subject in a dosage of anywhere between about 7.5 to about 1000 mg/m2. Further, 5-FU dosing schedules may be for a variety of time periods, for example up to six weeks, or as determined by one of ordinary skill in the art to which this disclosure pertains.

Gemcitabine diphosphate (GEMZAR®, Eli Lilly & Co., “gemcitabine”), another suitable chemotherapeutic agent, is recommended for treatment of advanced and metastatic pancreatic cancer, and will therefore be useful in the present disclosure for these cancers as well.

The amount of the chemotherapeutic agent delivered to the patient may be variable. In one suitable embodiment, the chemotherapeutic agent may be administered in an amount effective to cause arrest or regression of the cancer in a host, when the chemotherapy is administered with the construct. In other embodiments, the chemotherapeutic agent may be administered in an amount that is anywhere between 2 to 10,000 fold less than the chemotherapeutic effective dose of the chemotherapeutic agent. For example, the chemotherapeutic agent may be administered in an amount that is about 20 fold less, about 500 fold less or even about 5000 fold less than the chemotherapeutic effective dose of the chemotherapeutic agent. The chemotherapeutics of the disclosure can be tested in vivo for the desired therapeutic activity in combination with the construct, as well as for determination of effective dosages. For example, such compounds can be tested in suitable animal model systems prior to testing in humans, including, but not limited to, rats, mice, chicken, cows, monkeys, rabbits, etc. In vitro testing may also be used to determine suitable combinations and dosages, as described in the examples.

### J. Radiotherapy

In some embodiments, the treatment or prior therapy comprises radiation, such as ionizing radiation. As used herein, “ionizing radiation” means radiation comprising particles or photons that have sufficient energy or can produce sufficient energy via nuclear interactions to produce ionization (gain or loss of electrons). An exemplary and preferred ionizing radiation is an x-radiation. Means for delivering x-radiation to a target tissue or cell are well known in the art.

In some embodiments, the amount of ionizing radiation is greater than 20 Gy and is administered in one dose. In some embodiments, the amount of ionizing radiation is 18 Gy and is administered in three doses. In some embodiments, the amount of ionizing radiation is at least, at most, or exactly 2, 4, 6, 8, 10, 15, 16, 17, 18, 19, 20, 21, 22, 23, 24, 25, 26, 27, 18, 19, 30, 31, 32, 33, 34, 35, 36, 37, 38, 39, 40, 41, 42, 43, 44, 45, 46, 47, 48, 49, or 40 Gy (or any derivable range therein). In some embodiments, the ionizing radiation is administered in at least, at most, or exactly 1, 2, 3, 4, 5, 6, 7, 8, 9, or 10 does (or any derivable range therein). When more than one dose is administered, the does may be about 1, 4, 8, 12, or 24 hours or 1, 2, 3, 4, 5, 6, 7, or 8 days or 1, 2, 3, 4, 5, 6, 7, 8, 9, 10, 12, 14, or 16 weeks apart, or any derivable range therein.

In some embodiments, the amount of IR may be presented as a total dose of IR, which is then administered in fractionated doses. For example, in some embodiments, the total dose is 50 Gy administered in 10 fractionated doses of 5 Gy each. In some embodiments, the total dose is 50-90 Gy, administered in 20-60 fractionated doses of 2-3 Gy each. In some embodiments, the total dose of IR is at least, at most, or about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 125, 130, 135, 140, or 150 (or any derivable range therein). In some embodiments, the total dose is administered in fractionated doses of at least, at most, or exactly 1, 2, 3, 4, 5, 6, 7, 8, 9, 10, 12, 14, 15, 20, 25, 30, 35, 40, 45, or 50 Gy (or any derivable range therein. In some embodiments, at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 fractionated doses are administered (or any derivable range therein). In some embodiments, at least, at most, or exactly 1, 2, 3, 4, 5, 6, 7, 8, 9, 10, 11, or 12 (or any derivable range therein) fractionated doses are administered per day. In some embodiments, at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 (or any derivable range therein) fractionated doses are administered per week.

### K. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### VII. Pharmaceutical Compositions

In certain aspects, the compositions or agents for use in the methods, such as chemotherapeutic agents or biomarker modulators, are suitably contained in a pharmaceutically acceptable carrier. The carrier is non-toxic, biocompatible and is selected so as not to detrimentally affect the biological activity of the agent. The agents in some aspects of the disclosure may be formulated into preparations for local delivery (i.e. to a specific location of the body, such as skeletal muscle or other tissue) or systemic delivery, in solid, semi-solid, gel, liquid or gaseous forms such as tablets, capsules, powders, granules, ointments, solutions, depositories, inhalants and injections allowing for oral, parenteral or surgical administration. Certain aspects of the disclosure also contemplate local administration of the compositions by coating medical devices and the like.

Suitable carriers for parenteral delivery via injectable, infusion or irrigation and topical delivery include distilled water, physiological phosphate-buffered saline, normal or lactated Ringer's solutions, dextrose solution, Hank's solution, or propanediol. In addition, sterile, fixed oils may be employed as a solvent or suspending medium. For this purpose any biocompatible oil may be employed including synthetic mono- or diglycerides. In addition, fatty acids such as oleic acid find use in the preparation of injectables. The carrier and agent may be compounded as a liquid, suspension, polymerizable or non-polymerizable gel, paste or salve.

The carrier may also comprise a delivery vehicle to sustain (i.e., extend, delay or regulate) the delivery of the agent(s) or to enhance the delivery, uptake, stability or pharmacokinetics of the therapeutic agent(s). Such a delivery vehicle may include, by way of non-limiting examples, microparticles, microspheres, nanospheres or nanoparticles composed of proteins, liposomes, carbohydrates, synthetic organic compounds, inorganic compounds, polymeric or copolymeric hydrogels and polymeric micelles.

In certain aspects, the actual dosage amount of a composition administered to a patient or subject can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

Solutions of pharmaceutical compositions can be prepared in water suitably mixed with a surfactant, such as hydroxypropylcellulose. Dispersions also can be prepared in glycerol, liquid polyethylene glycols, mixtures thereof and in oils. Under ordinary conditions of storage and use, these preparations contain a preservative to prevent the growth of microorganisms.

In certain aspects, the pharmaceutical compositions are advantageously administered in the form of injectable compositions either as liquid solutions or suspensions; solid forms suitable or solution in, or suspension in, liquid prior to injection may also be prepared. These preparations also may be emulsified. A typical composition for such purpose comprises a pharmaceutically acceptable carrier. For instance, the composition may contain 10 mg or less, 25 mg, 50 mg or up to about 100 mg of human serum albumin per milliliter of phosphate buffered saline. Other pharmaceutically acceptable carriers include aqueous solutions, non-toxic excipients, including salts, preservatives, buffers and the like.

Examples of non-aqueous solvents are propylene glycol, polyethylene glycol, vegetable oil and injectable organic esters such as ethyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, antgifungal agents, anti-oxidants, chelating agents and inert gases. The pH and exact concentration of the various components the pharmaceutical composition are adjusted according to well-known parameters.

Additional formulations are suitable for oral administration. Oral formulations include such typical excipients as, for example, pharmaceutical grades of mannitol, lactose, starch, magnesium stearate, sodium saccharine, cellulose, magnesium carbonate and the like. The compositions take the form of solutions, suspensions, tablets, pills, capsules, sustained release formulations or powders.

In further aspects, the pharmaceutical compositions may include classic pharmaceutical preparations. Administration of pharmaceutical compositions according to certain aspects may be via any common route so long as the target tissue is available via that route. This may include oral, nasal, buccal, rectal, vaginal or topical. Topical administration may be particularly advantageous for the treatment of skin cancers, to prevent chemotherapy-induced alopecia or other dermal hyperproliferative disorder. Alternatively, administration may be by orthotopic, intradermal, subcutaneous, intramuscular, intraperitoneal or intravenous injection. Such compositions would normally be administered as pharmaceutically acceptable compositions that include physiologically acceptable carriers, buffers or other excipients. For treatment of conditions of the lungs, aerosol delivery can be used. Volume of the aerosol is between about 0.01 ml and 0.5 ml.

An effective amount of the pharmaceutical composition is determined based on the intended goal. The term “unit dose” or “dosage” refers to physically discrete units suitable for use in a subject, each unit containing a predetermined-quantity of the pharmaceutical composition calculated to produce the desired responses discussed above in association with its administration, i.e., the appropriate route and treatment regimen. The quantity to be administered, both according to number of treatments and unit dose, depends on the protection or effect desired.

Precise amounts of the pharmaceutical composition also depend on the judgment of the practitioner and are peculiar to each individual. Factors affecting the dose include the physical and clinical state of the patient, the route of administration, the intended goal of treatment (e.g., alleviation of symptoms versus cure) and the potency, stability and toxicity of the particular therapeutic substance.

### VIII. Kits

Certain aspects of the present invention also concern kits containing compositions of the invention or compositions to implement methods of the invention. In some embodiments, kits can be used to evaluate one or more biomarkers. In certain embodiments, a kit contains, contains at least or contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 500, 1,000 or more probes, primers or primer sets, synthetic molecules, antibodies, or inhibitors, or any value or range and combination derivable therein. In some embodiments, there are kits for evaluating biomarker activity or level in a cell.

Kits may comprise components, which may be individually packaged or placed in a container, such as a tube, bottle, vial, syringe, or other suitable container means.

Individual components may also be provided in a kit in concentrated amounts; in some embodiments, a component is provided individually in the same concentration as it would be in a solution with other components. Concentrations of components may be provided as 1×, 2×, 5×, 10×, or 20× or more.

Kits for using probes, antibodies, synthetic nucleic acids, nonsynthetic nucleic acids, and/or inhibitors of the disclosure for prognostic or diagnostic applications are included as part of the disclosure. Specifically contemplated are any such molecules corresponding to any biomarker identified herein, which includes antibodies that bind to such biomarkers as well as nucleic acid primers/primer sets and probes that are identical to or complementary to all or part of a biomarker, which may include noncoding sequences of the biomarker, as well as coding sequences of the biomarker.

In certain aspects, negative and/or positive control nucleic acids, antibodies, probes, and inhibitors are included in some kit embodiments. In addition, a kit may include a sample that is a negative or positive control for methylation of one or more biomarkers.

It is contemplated that any method or composition described herein can be implemented with respect to any other method or composition described herein and that different embodiments may be combined. The claims originally filed are contemplated to cover claims that are multiply dependent on any filed claim or combination of filed claims.

### IX. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1: Whole Transcriptome Sequencing of Patients' Whole Blood with Hemoglobin Depletion Reveals Potential Glioblastoma Biomarkers: A Pilot Prospective Study

Glioblastoma multiforme (GBM) is the most aggressive brain cancer in adults. The diagnosis, prognosis, and treatment monitor of GBM currently rely on imaging and invasive brain biopsies. There are few minimally invasive blood-derived biomarkers for GBMs implemented in clinical setting. The aim of this study is to develop an integrated method to accurately detect the differential gene expression (DGE) from patient whole blood specimens, and to identify potential GBM blood biomarkers from a pilot prospective cohort.

Whole blood samples from GBM patients and non-GBM individuals as controls were collected at Baylor Scott & White Heath. After total RNA extraction, depletion of hemoglobin mRNAs was conducted prior to RNA sequencing. Multi-faceted analyses were performed to identify the most differentially expressed genes and microRNAs (DEGs and DEMs) in GBM blood. Subsequent validation was performed on additional whole blood and tissue specimens by quantitative reverse transcription-PCR.

DGE analysis of the sequencing result identified 487 DEGs with fold change (FC)≥2.0 at false discovery rate (FDR)≤0.05, and 30 DEMs with FC≥2.0 at p-value ≤0.05. KEGG pathway enrichment analysis revealed HIF-1 signaling, leukocyte transendothelial migration, pathways in cancer, etc. as most perturbed pathways in GBM blood. Gene Ontology analysis revealed that transcription regulation, apoptotic process, protein phosphorylation, immune response, etc. are well represented in GBM blood. After integrated analysis, 90 GBM related DEGs were pulled out. By RT-qPCR validation, 5 genes and 1 microRNA (miRNA) were found upregulated, and 4 genes and 1 miRNA were found downregulated in >50% testing GBM blood samples in comparison with controls. By building a logistic regression model using blood RNA-sequencing data, the PCR results of the genes were predicted to have high accuracy in additional independent patient blood.

Globin reduction markedly increases the sensitivity of DGE detection using RNA-sequencing in human whole blood samples and allows a panel of 11 gene candidates to be identified as potential blood biomarkers for GBM patients.

Key Objective: To develop an integrated method to accurately detect the differential gene expression using minimally invasive liquid biopsy (here patient whole blood specimens), and to identify potential glioblastoma (GBM) blood biomarkers from a pilot prospective cohort.

Knowledge Generated: Globin reduction markedly increases the sensitivity of DGE detection using RNA-sequencing in human whole blood samples and allows the identification of a pool of differentially expressed genes (DEGs) and differentially expressed miRNAs (DEMs) between non-GBM group and GBM group.

Relevance: Although further validation is needed, by using the combined approach of globin depletion and transcriptome RNA sequencing, the inventors enriched the differential expression of 11 genes, the expression configuration of which is likely a common trait of GBM blood and has the potential to be developed as GBM blood biomarker.

### Introduction

Glioblastoma mutiforme (GBM) is the most aggressive malignant brain glioma (WHO grade IV) which occurs in human central nervous system (CNS) with a poor survival (1-3). Current treatment options for GBM patients are surgical resection, radiation, chemotherapy, and tumor treating fields (TTF) (4,5). The diagnosis, treatment efficacy monitor, and clinical assessment of GBM prognosis rely on magnetic resonance imaging (MRI), computed tomography (CT) scan, and invasive open biopsy. Despite the development of marker discovery techniques in liquid biopsy such as circulating tumor cells (CTCs), cell free DNA/RNA (cfDNA/cfRNA), extracellular vesicles, cerebrospinal fluid (CSF), and platelets (6-10), there are still few effective GBM specific biomarkers applying in clinical practice, especially blood-derived markers. To help improve the survival of GBM patients, more minimally invasive markers for early diagnosis, real-time monitor of treatment effect and accurate prognostication are in pressing need.

Patient blood are attractive media for the identification of disease biomarkers because of their critical roles in immune response, metabolism, communications with cells, and formation of extracellular matrices in various tissues, organs in the human body, as well as the simplicity and less invasive nature of sample collection (18-21). However, few biomarker studies have been focused on the direct use of whole blood specimen for GBM patients.

The efforts to identify biomarkers from blood in cancer have not been satisfactory so far. The major obstacle for marker discovery in whole blood is the abundant amount of hemoglobin. The inventors have developed an efficient approach for the identification of markers in whole blood to circumvent the problem (39). The major obstacle for marker discovery in whole blood is the abundant amount of hemoglobin. The inventors have clearly shown that globin reduction (GR) improved the detective sensitivity of gender marker identification (39). In this study, the inventors aim to seek for the gene/miRNA expression differences in whole blood comparing GBM and control samples by using the integrated strategy of globin removal and RNA-sequencing (RNA-seq) as well as gene analytical tools. Here, the inventors demonstrate that GR increases the sequencing reads of non-globin genes in whole blood samples and therefore improves the detection sensitivity of differential gene expression (DGE). The inventors have identified 487 differentially expressed genes (DEGs) with fold change (FC) over 2.0 at false discovery rate (FDR) 0.05 from mRNA sequencing (mRNA-seq), and 30 differentially expressed miRNAs (DEMs) with FC≥2.0 and p-value ≤0.05 from small RNA sequencing (small RNA-seq). After comprehensive data analysis and validation by the means of quantitative real time reverse transcription PCR (RT-qPCR), a panel of 11 gene candidates were identified as potential GBM blood biomarkers.

### B. Patients and Methods

### 1. Patients and Experiment Design

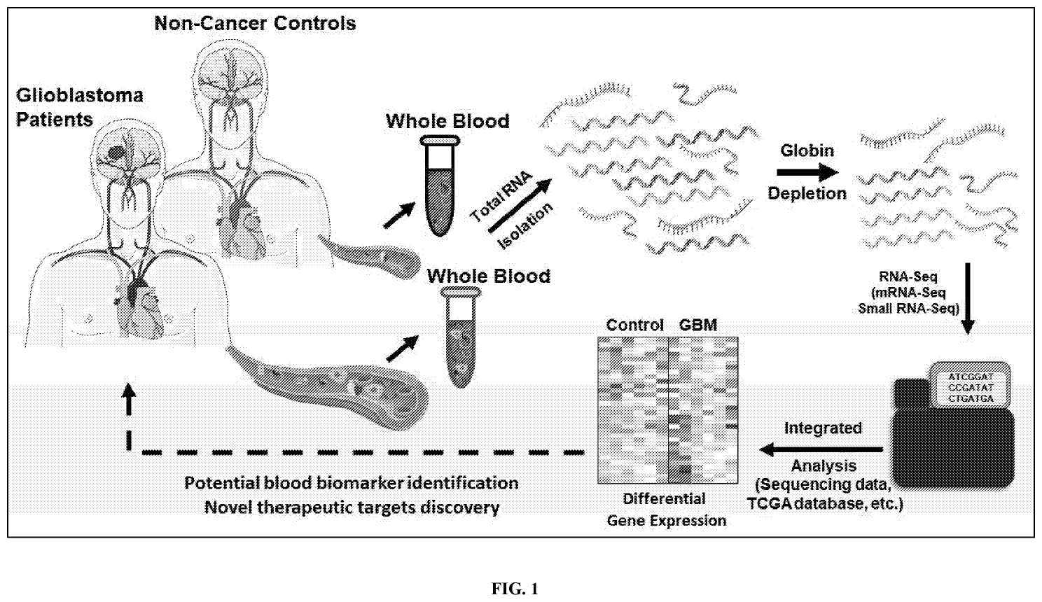

Total 15 GBM patients and 14 non-GBM control individuals were recruited for blood sample collection. Clinical details of these human subjects including samples for RNA-sequencing and RT-qPCR was provided in Supplemental Table S1. In addition, ten tumor tissues from GBM patients and two normal adjacent tissues were obtained from Baylor Scott & White Neuroscience Institute Brain Bank. Tissues obtained during surgery was snap frozen in liquid nitrogen and then stored at −80° C. till use. The clinical information of tissue samples used for data validation was also provided in Supplemental Table S1. GBM was diagnosed by board-certified neuropathologist based tumor histology. Treatment of patient was administered by board-certified neuro-oncologists. Patients and control individuals all voluntarily agreed to participate in this study and written informed consent forms were obtained from the participants or their proxies. The clinical demographic characteristics of patients are summarized in Table 1. The overall experimental design is depicted in FIG. 1. This study was conducted under the ethic approval of the BSWH Institutional Review Board.

### 2. RNA Preparation

For whole blood specimens, peripheral blood from participants was drawn with a BD safely Lok™ blood collection set (BD, Franklin Lakes, NJ) into PAXgene RNA collection tube (Qiagen) according to the manufacturer's guidance and then kept at −80° C. Total RNAs (including small RNAs) were isolated with the PAXgene Blood miRNA Kit (Qiagen) based on the manufacturer's protocol with an additional on-column DNase digestion step. For brain tissue specimens, total RNAs including small RNAs were extracted using RNeasy Mini Kit (Qiagen) following the manufacturer's instruction. The quantity of extracted total RNAs were determined using NanoDrop Spectrophotometer (NanoDrop Technologies). The quality of RNAs were evaluated using the 2100 Bioanalyzer system (Aligent).

### 3. Globin Reduction

The GLOBINclear™ Kit (Ambion) was employed to remove the highly abundant hemoglobin mRNAs from the blood isolated total RNA samples according to the manufacturer's instruction. Briefly, 1 μg total RNAs from each sample were hybridized with a biotinylated Capture OLIGO Mix which is specific for human mRNA hemoglobin a and b. Streptavidin Magnetic Beads were added to bind the biotinylated oligonucleotides that hybridized with globin mRNAs and remaining RNAs were then pulled down by magnet. The globin depleted RNAs were transferred to a fresh tube and further purified with a rapid magnetic bead-based purification process. The resulting samples were stored at −80° C. before use.

4. Library Construction and RNA-Sequencing for mRNAs

RNA samples both pre and post-globin reduction with a RNA integrity number (RIN)≥7.0 were used for cDNA paired-end library construction. First, mRNA molecules were purified from total RNA using oligo (dT)-attached magnetic beads. Then mRNAs were fragmented into small pieces. First-strand cDNA was generated using random hexamer-primed reverse transcription, followed by a second-strand cDNA synthesis. Then PCR was performed and PCR products were purified with AMPure XP beads (Agencount, Beckman Coulter) and library quality was validated on the 2100 Bioanalyzer system (Agilent). The double stranded PCR products were heat denatured and circularized by the splint oligo sequence. The single strand circle DNA (ssCir DNA) were formatted as the final library. The libraries were sequenced on an Illumina Hiseq2000 platform at Beth Israel Deaconess Medical Center (BIDMC) Genomics Proteomics Core in Harvard Medical School and 100 bp paired-end reads were generated.

### 5. Small RNA Library Construction and Sequencing

Small RNA library construction and sequencing were performed at BIDMC Genomics Proteomics Core. Briefly, approximately 1 μg RNA of an RIN≥7.0 from each sample was used. RNA segments of different size were separated by PAGE gel, 18-30 nt stripes were selected and recycled. Then 3′ adaptor connection system, RT primer addition and 5′ adaptor connection system were prepared. After that, strand cDNA synthesis, PCR amplification, library fragment selection were performed. The double stranded PCR products were heat denatured and circularized by the splint oligo sequence. The single strand circle DNA (ssCir DNA) were formatted as the final library. Library was validated on the 2100 Bioanalyzer system (Agilent). Then the libraries were sequenced on a BGISEQ-500 platform (Beijing Genomics Institute, China) at Beth Israel Deaconess Medical Center (BIDMC) Genomics Proteomics Core in Harvard Medical School.

### 6. Quality Control and Bioinformatic Analysis of RNA-Seq Data

mRNA-Seq generated an average of ˜87 million raw reads for each sample. After quality filtering by removing reads containing adapters, Poly-N or have low quality from the total raw reads, an average of 43.7 million high-quality clean reads were obtained for each sample. The small RNA-seq yielded an average of 34.27 million raw reads for each sample, and 23.05 million (67%) clean reads were retained. The quality characteristics of sequence reads for each sample is over 97%. Q20 for each sample is over 97%, GC contents are around 49% to 59% for sequenced samples. Quality control for raw sequencing reads was performed by the FastQC software (40). Reads from mRNA-seq were aligned to human reference genome (GRCh38) using hisat2 (41). 97.6% of the clean reads from mRNA-seq and 89% of the clean reads from small RNA-seq were mapped to the human genome. SAM files obtained from the aligner were converted to BAM format using SAMtools (42). FeatureCounts (43) was used to quantify the total number of counts for each gene. The small RNA sequencing reads were aligned and counted using the extra-cellular RNA processing toolkit (exceRpt) (44). Briefly, the pipeline first filtered the reads that mapped to UniVec vector and ribosomal RNA sequences and the unmapped reads were then aligned to the human genome (hg38) and quantified for different types of RNAs including miRNAs (miRBase v21 (http://www.mirbase.org/)) and other small RNAs.

7. DGE Analysis, miRNA-mRNA Integrative Analysis and Data Visualization

Both RNA-Seq and small RNA-Seq read counts were normalized using the median-of-ratios method (45) and log 2 transformed for data visualization. Differential gene expression analysis was performed with DESeq2 package in R (46); genes that survived FDR≤0.05 or p-value ≤0.05 were considered differentially expressed. Heatmaps were plotted using the ClustVis webtool (47) or the NMF package in R (48). Hierarchical clustering of representative mRNA and miRNA expressions were performed to reveal reproducibility in biological replicates.

The predicted target genes of DEMs were downloaded from the TargetScan database (49, 50). The overlap of predicted target genes and DEGs were identified and used for functional analysis, which was performed with the Database for Annotation, Visualization and Integrated Discovery (DAVID) v6.8 (51). Gene Ontology (GO) (http://www.geneontology.org) analysis was performed to help elucidate the over-represented biological functions of specific genes, and KEGG pathway (http://www.genome.jp/kegg) enrichment was employed to identify the canonical signal pathways. GO biological process (BP) terms and KEGGs pathways with p-value ≤0.05 were considered significantly enriched. The miRNA-mRNA network were generated using Cytoscape (52).

### 8. DGE Analysis of GBM Tissue Datasets and Data Visualization

The Cancer Genome Atlas (TCGA) (53) database was used to download the transcriptome RNA profiling data for 156 primary GBM tissues and 5 solid normal tissue controls (54), the case IDs used in this work have been listed in Supplemental Table S2. DGE analysis was performed with DESeq2 package in R (46); genes that survived FDR 0.05 were considered differentially expressed. Heatmaps were plotted using the NMF package in R (48). The generated DEG list was compared with the blood RNA-seq data to identify the overlapped genes.

R2: Genomics Analysis and Visualization Platform was also used to analyze gene expression difference of GBM tissues compared to normal brain tissues. Two public datasets of normal brain tissues (“Normal Brain PFC-Harris-44-MAS5.0-u133p2” (55) and “Normal Brain regions-Berchtold-172-MAS5.0-u133p2” (56)) and three public datasets of GBM tumor tissues (“Tumor Glioblastoma-Hegi-84-MAS5.0-u133p2” (57), Tumor Glioblastoma-Loeffler-70-MAS5.0-u133p2” (58) and “Tumor Glioblastoma-Pfister-46-MAS5.0-u133p2” (59)) were selected and analyzed through R2: megasearch online portal. Gene expression differences were considered significant at FDR≤0.01. The generated DEGs was also compared with the blood RNA-seq data to identify the overlapped genes.

### 9. Baylor Module Gene Enrichment Analysis

To facilitate interpretation of the gene expression signature, the inventors used a pre-existing framework of 260 transcriptional modules to analyze this dataset (60, 61). For each module, the percentage of transcripts significantly up- or down-regulated was calculated, and the module score was defined as the difference in percent up or down. For example, if a module contains 100 transcripts in which 60 are significantly up-regulated and 20 are significantly down-regulated, the module score would be 0.60-0.20=0.40 and would have a mild red color. Data was considered significance at FDR≤0.05 and visualized using R package.

### 10. RT-qPCR Analysis

Total RNAs including small RNAs were extracted from tumor tissues and blood of GBM patients or control counterparts using miRNeasy Mini Kit (Qiagen) and PAXgene Blood miRNA Kit (Qiagen), respectively. For miRNA detection, 100 ng total RNAs was used to complete reverse transcription and PCR with the miScript PCR system including miScript II RT Kit, miScript Primer Assays and miScript SYBR Green PCR Kit (Qiagen), according to the manufacturer's instructions. For mRNA detection, 100 ng of total RNA was used to prepare cDNA using iScript Reverse Transcription Supermix (Bio-Rad) by following the manufacturer's instruction, and then subjected to qPCR assay. RT-qPCR was performed using iTaq Universal SYBR Green Supermix (Bio-Rad). All the qPCR assays were performed in triplicate experiments on CFX96 Touch™ Real-Time PCR Detection System. Cycle threshold (Ct) values were calculated using the automated settings of the system. PCR amplification was done following the manufacturer's instructions. Fold change obtained from Ct values using 2^{-ΔΔCt} methodology⁶² was converted into logarithmic base 2 for statistical analysis. P-values≤0.05 were considered to be statistically significant. Human GAPDH, U6 and 18S rRNA were used as the internal controls for mRNAs, miRNAs and long non-coding RNAs of interest, respectively. Gene specific primers were designed using Primer-BLAST online tool. The primer sequences are listed in Supplemental Table S3. Data were presented as mean±standard derivation (SD) in percentages.

### 11. Statistical Analysis

The level of significance for gene expression difference between control group and GBM group was analyzed by the moderated t-statistic in datasets analysis for RNA-seq data using the DESeq2 package in R or student t-test for RT-qPCR data using Graphpad Prism 7.0. Statistical significance was defined as FDR≤0.05 or p-value 0.05 according to the analysis.

### C. Results

1. Globin mRNA Reduction Improves Informative Reads

To identify potential biomarker(s) for GBM patients through gene expression profile comparison between non-GBM individuals and GBM patients by the means of deep RNA-seq. Whole blood samples from 12 non-GBM controls and 10 GBM patients collected at Department of Neurology, BSWH, USA were used. The demographic clinical characteristics is shown in Table 1. The major obstacle for marker discovery in whole blood is the existence of abundant amount of hemoglobin. In a previous publication, the inventors have shown that globin reduction significantly improved the detective sensitivity of gender marker identification on microarray (39). Therefore, here the inventors first isolated total RNAs including small RNAs from the collected whole blood samples and then performed globin depletion prior to subsequent mRNA-seq and small RNA-seq.

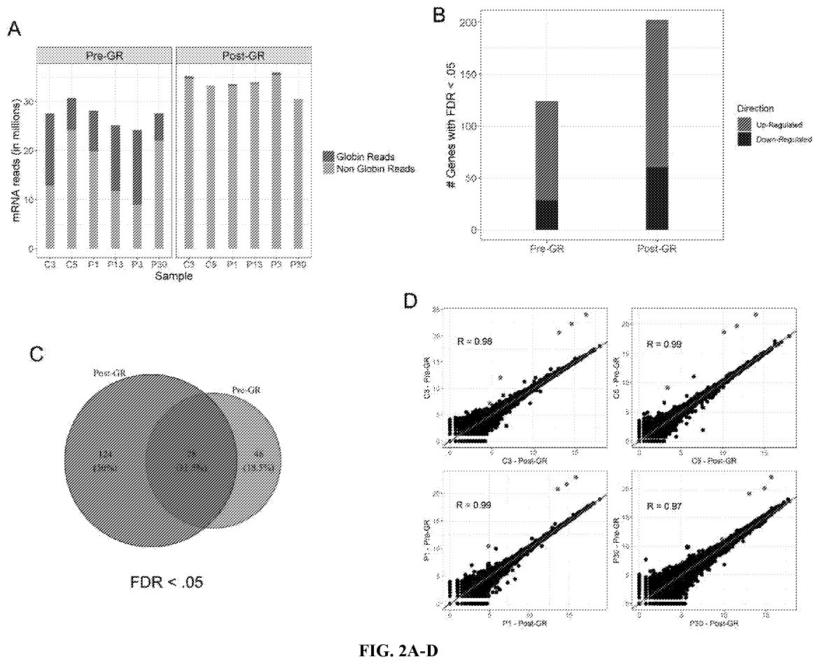

To assess the effect of globin depletion before RNA-seq, half of the prepared RNAs from five GBM patients and two controls were left as pre-GR controls and all of the rest RNAs were subjected to globin removal. As shown in FIG. 2A, GR reduced the percentage of total reads dominated by globin genes from 20-63% per sample to less than 1.5% for all samples. Meanwhile, GR increased the number of non-globin mRNA reads by an average of over two fold for both upregulated and downregulated genes (FIG. 2B). In addition, DGE analysis comparing GBM versus controls identified 31.5% more DEGs (FDR≤0.05) in post-GR samples than pre-GR samples (FIG. 2C). The concordance of gene expression level of a given sample was evaluated using scatter plots showing the comparison of pre-GR and post-GR. The high correlation coefficient of concordance with R≥0.97 (FIG. 2D) suggests that GR didn't introduce bias to the expression detection of non-globin genes. Collectively, the inventors here demonstrates that GR step before high throughput RNA-seq process increases the detection sensitivity and improves the informative reads.

### 2. DGE and Functional Enrichment Analysis of RNA-Seq Data Reveal Significantly Changed Genes and Perturbed Pathways in GBM Blood

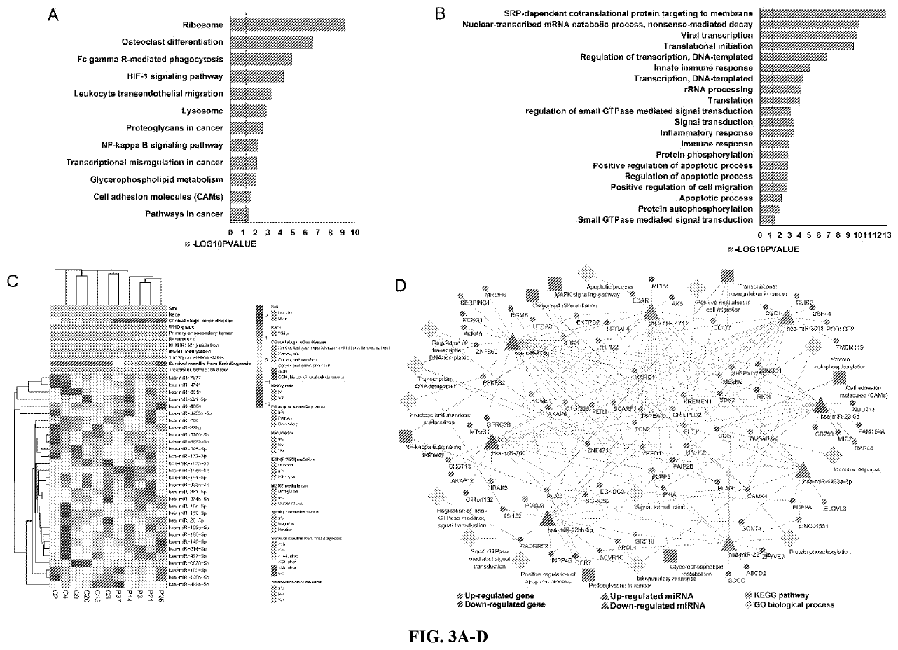

To analyze the gene expression profiling differences between control and GBM groups, sequencing data of post-GR samples were pooled together and analyzed. Differential expression analysis was performed. Comparisons were performed between GBM and control groups with the adjustment for gender. mRNAs with FDR≤0.05 and miRNAs with p-value≤0.05 were considered differentially expressed. DEGs and DEMs with |log 2FC|≥1.0 were selected and used to further analysis and data visualization. As a result, 487 DEGs (250 up, 237 down) with |log 2FC|≥1.0 and FDR≤0.05, and 30 DEMs (7 up, 23 down) with |log 2FC|≥1.0 and p-value≤0.05 were identified. Among those DEGs, MMP9, TMEM92, C1orf226, CD163, SH3PXD2B, etc. were predominantly increased in detected GBM patients, whereas AK5, CCR7, CD200, MICU3, BEX2, etc. were decreased. Among the identified DEMs, miR-3918, miR-221-3p, etc. were up-regulated, whereas miR-760, miR-125b-5p, miR-1299, etc. were down-regulated. To further characterize the main biological function and key pathways of the DEGs, functional enrichment was performed by DAVID for KEGG pathway and gene ontology (GO) analysis and ordered according to computed p-values. As shown in FIGS. 3A and B, KEGG pathway enrichment showed that, changes of pathways in cancer, leukocyte endothelial migration, lysosome pathways, proteoglycans in cancer and HIF-1 signaling pathway and so on, are involved in GBM. At the same time, GO BP term analysis showed that alterations of transcription and its regulation, protein phosphorylation and transportation, autophagy, apoptotic process, and signal transduction and so on, are involved in GBM. These observations suggest there are some common alterations of signaling pathways and biological processes among cancers.

3. miRNA Target Gene Prediction and Integrative Analysis Reveals Possible Gene Regulation Network

To analyze the possible functional gene network in GBM patients, potential target genes of identified DEMs were predicted and possible gene network connecting DEMs and their regulated genes was also visualized. Potential target genes of the 30 DEMs (FIG. 3C) were predicted using TargetScan v7.1 and miRWalk database. More than 1900 overlapped genes were obtained between predicted target genes and the identified DEGs at FDR≤0.05, FC of 179 genes amid them were over 2.0. With the consideration of FC and commonality in GBM group, a functional regulatory network was generated (FIG. 3D). Notable genes involved in the enriched KEGG pathways and GO BP terms were indicated.

### 4. DGE Analysis of TCGA and R2 Tissue Databases Reveals Circulating Genes in GBM Blood.

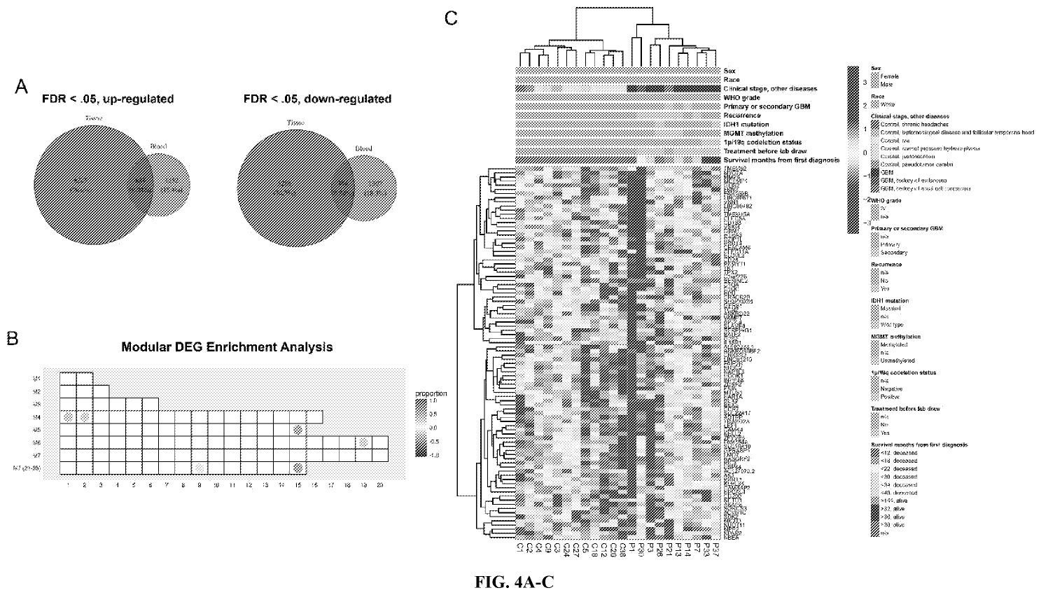

To assess the correlation of gene expression in whole blood and tissue samples, the inventors accessed and analyzed the public available tissue gene expression profiling data. The Cancer Genome Atlas (TCGA) (53) database was firstly used to download GBM RNA-Seq tissue data for 156 primary GBM tissues and 5 solid normal tissue controls. Differential analysis comparing GBM tumor tissues with normal tissues was performed. Consequently, overlapped DEGs were found from those identified from GBM tissue (FDR≤0.05) and the DEGs identified from GBM blood. A total of 12651 DEGs (FDR≤0.05) were identified (FIG. 4A). R2 Genomics Analysis and Visualization Platform was also used to identify potential DEGs in GBM tissue microarray datasets, two normal brain tissue datasets and three GBM tumor tissue datasets were compared using R2 online tool (r2.amc.nl). A total of 16040 potential DEGs (FDR≤0.01) were identified.

Comparison of the blood DEGs identified from RNA-seq and tissue DEGs identified from both TCGA and R2 databases were performed, and 73 overlapped DEGs with FC over 2.0 were obtained. This result indicates that whole blood gene expression partially reflects GBM tissue feature, which reveals the existence of circulating molecules in GBM blood and strongly supports the possibility of GBM blood biomarker identification.

### 5. Baylor Module Analysis Facilitates DEG Enrichment

To facilitate interpretation of the gene expression signature, Baylor module analysis was conducted to generally perform DEG enrichment for whole blood sequencing data. From a previous blood gene modular analysis at the inventors' own research institute, gene subsets divided into various modules were established for blood gene expression profiling analysis (60, 61). The DEGs identified with a |log 2FC|≥1.0 were subjected to module enrichment analysis. As shown in FIG. 4B, differences were observed at modules M4.1, M4.2, M5.15, M6.19, M7.29 and M7.35, genes in which were selected for further analysis. Among those modules, M4.1 shows genes involved in T cell function, M4.2 shows genes involved in inflammation and M5.15 show genes linked to Neutrophils, which are all related to immune response.

Collectively, after the integrated analyses of predicted miRNA target gene data, GBM tissue data, and Baylor modular data, a final 90 DEGs were pulled out and summarized with patient clinical characteristics in FIG. 4C. Gene heatmap was generated using blood RNA-seq data by the ClustVis webtool (47).

### 6. RT-qPCR Validation in Newly Collected Whole Blood and Tissue Specimens Elicits a Panel of GBM Related Blood Marker Candidates

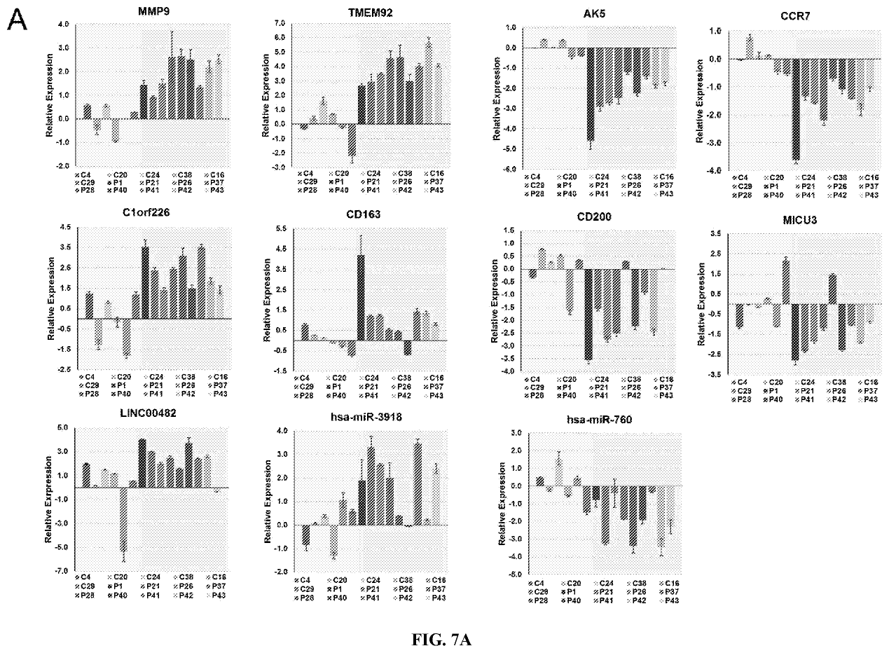

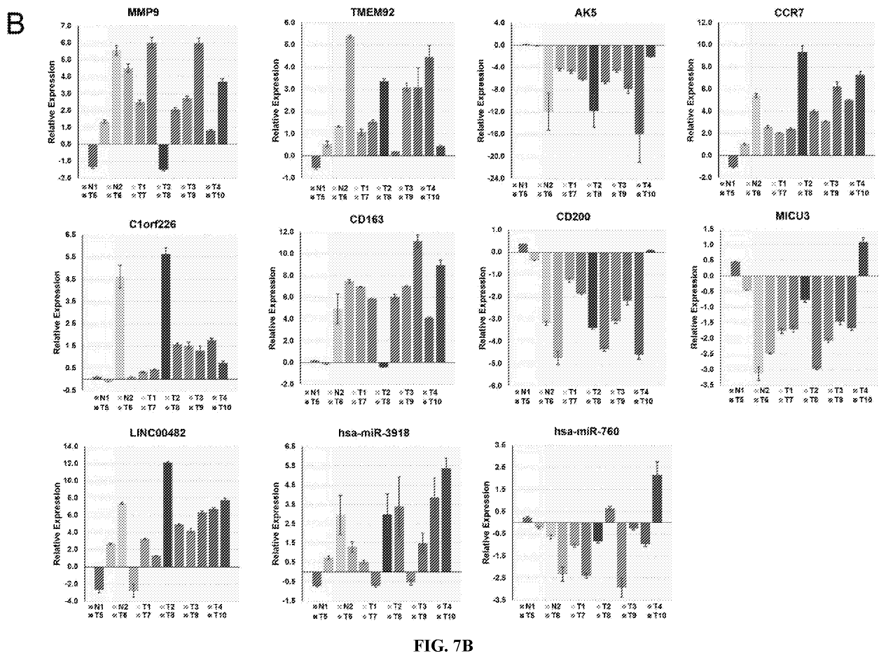

To validate the findings based on RNA-seq data, whole blood samples from two non-GBM individuals (designated as C16 and C29) and five GBM patients (designated as P28, P40, P41, P42, and P43) were further collected at BSWH. The inventors selected nigh DEGs and two DEMs to perform RT-qPCR experiments from the afore-described results. Log 2 (FC) values were transformed based on the ratios between GBM and control group average expression values. Blood samples analyzed in RNA-seq from control individuals (C4, C20, C24 and C38) and GBM patients (P1, P21, P26, and P37) were used as methodological positive control. As shown in FIG. 7A, the gene expression pattern of these samples in RT-qPCR data were consistent with RNA-seq data, which validated the reliability of the sequencing data and RT-qPCR results. MMP9 and TMEM92 were up-regulated in all five testing GBM blood samples (P28, P40, P41, P42, and P43), AK5 and CCR7 were down-regulated in 100% (all 5) and 80% (4 out of 5) of the testing GBM blood samples, respectively. Higher expression levels of C1orf226 and long noncoding RNA LINC00482 were observed in 60% (3 out of 5) of testing GBM blood. CD200 and MICU3 expression difference were seen in 40% (2 out of 5) of the testing GBM samples. For miRNAs, as C24, C38 and P1 were not used in miRNA-seq, they were considered as testing samples in miRNA validation. As a result showing in FIG. 7A, hsa-miR-3918 upregulation was observed in 50% (3 out of 6) of the testing GBM blood samples, hsa-miR-760 downregulation was also observed in 66.7% (4 out of 6) of the testing GBM samples.

To evaluate the expression of these genes in brain tissue, tumor tissues (designated as T1-T10) and normal adjacent tissues of T1 and T2 (designated as N1 and N2) from GBM patients were obtained from Baylor Scott and White Neuroscience Institute Brain Bank, PCR results were shown in FIG. 7B. MMP9, TMEM92, CD163, C1orf226, and LINC00482 expressions are higher in at least 70% of the tumor tissues compared to normal tissues, meanwhile, AK5, CD200 and MICU3 expressions are lower in at least 80% of the tumor tissues compared to normal controls. Unlike the result in blood, CCR7, which is not specifically expressed in brain, is upregulated in tumor tissues in comparison with normal controls, indicating the local difference of immune response. For miRNA, the expression of hsa-miR-3918 are higher in 70% of the tumor tissues and hsa-miR-760 expression are lower in 60% of the GBM tumors compared with normal tissues. Furthermore, the inventors trained a logistic regression model using the 9 DEGs on 22 RNA-Seq samples (12 controls, 10 GBMs). The training data was first normalized using DESeq2 and log 2 transformed. To make the training data more comparable with the delta qPCR data, the inventors further normalized by GAPDH and centered and scaled each DEG. Finally, the inventors validated the trained model on the seven independent newly collected blood samples (2 controls, 5 GBM) and achieved 100% accuracy.

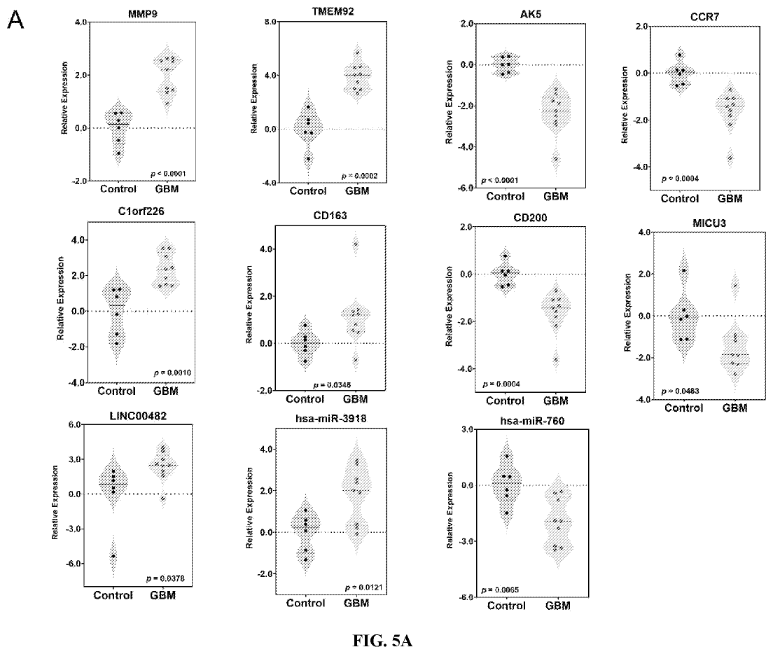

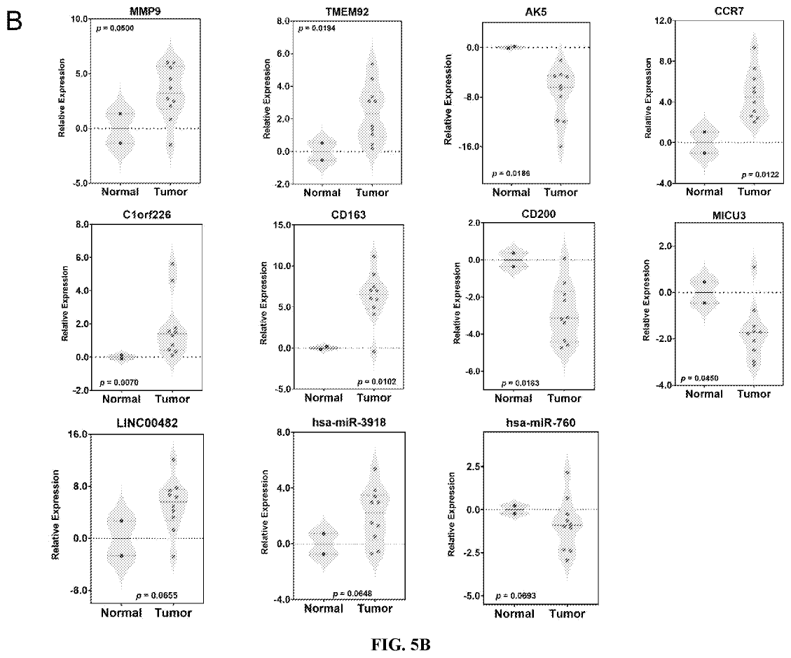

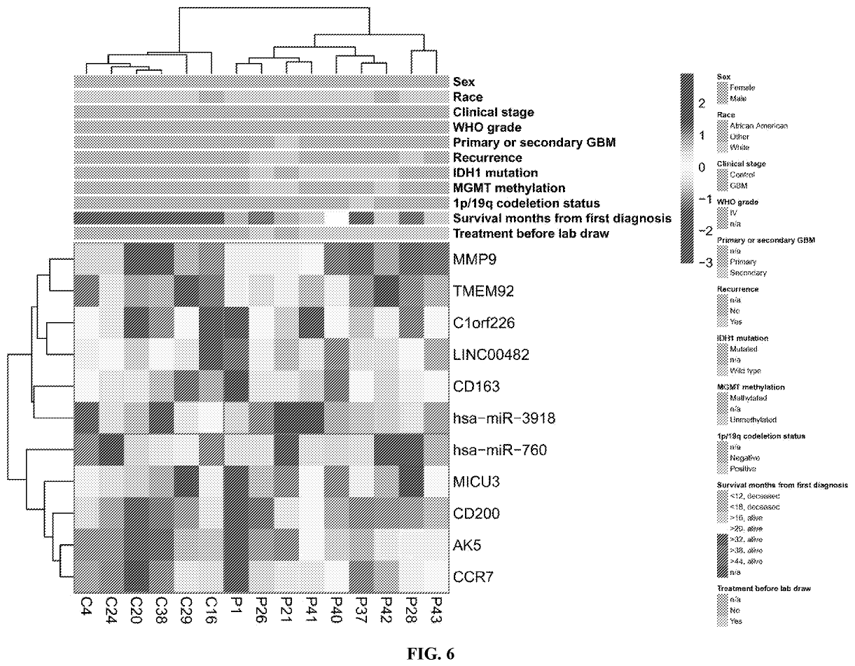

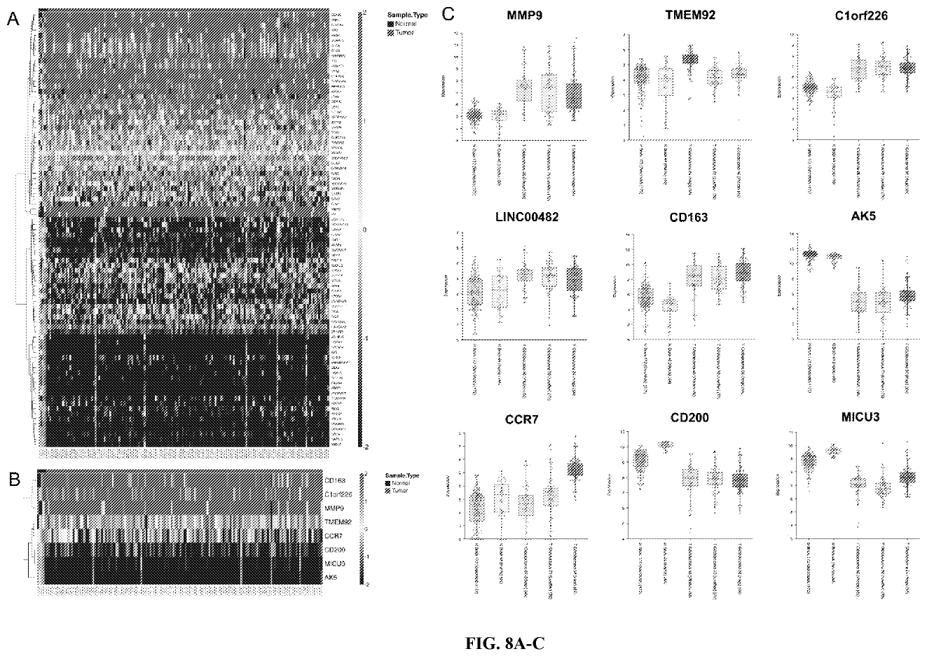

The inventors summarized the PCR results and significance by comparing GBM group with control group using student t-test and shown in FIGS. 5A and 5B. qPCR data from GBM blood samples were also visualized using heatmaps generated by ClustVis tool with individual clinical information (FIG. 6). Genes were also analyzed using extracted TCGA GBM transcriptome profiling data and R2 Genomics database in GBM cohorts, the data were summarized in FIG. 8, which shows significant expression difference of MMP9, C1orf226, CD163, LINC00482, AK5, MICU3 and CD200 in tumor tissue data. TCGA data of the all 90 enriched genes in blood RNA-seq results were also analyzed and showed in FIG. 8. Therefore, 11 potential GBM blood markers are proposed from this work including the expression pattern of 8 genes, 1 long non-coding RNA and 2 miRNAs.

### D. Discussion

In this study, the inventors present an integrated approach to identify GBM signature by gene-expression profiling of whole blood with globin depletion. Here, the inventors see the benefit of globin depletion on increasing the informative reads from RNA-seq and detective sensitivity of DGE. In total, the inventors identified 487 DEGs with FC≥2.0 and FDR≤0.05, and 30 DEMs with FC≥2.0 and p-value ≤0.05. KEGG pathway enrichment analysis of the DEGs reveals that HIF1 signaling, NF-κB signaling, leukocyte endothelial migration pathways in cancer, and proteoglycans, etc. are perturbed in GBM. The result of GO enrichment analysis show that transcription regulation, immune response, inflammatory response, apoptotic process, protein phosphorylation, etc. are altered in GBM. Some alterations reflected the molecular characteristics in tumor tissue. Moreover, through merging analysis of DEMs' predicted target genes and DEGs, tissue database analysis from TCGA and R2 genomics, and Baylor modular gene enrichment analysis, a final 90 DEGs were elicited and 11 blood marker candidates including DEMs were validated in newly collected additional whole blood samples and tissue specimens by RT-qPCR. Specifically, MMP9, TMEM92, C1orf226, CD163, LINC00482 and hsa-miR-3918 upregulation, AK5, CCR7, CD200, MICU3, and hsa-miR-760 downregulation in GBM. Among them, genes such as TMEM92, C1orf226, AK5, MICU3, and has-miR-3918 have not been previously reported to be associated with GBM before. Furthermore, by the means of logistic regression model building using RNA-seq data, the qPCR results of the marker expression achieved 100% accuracy in newly collected independent blood samples.

GBM, WHO grade IV glioma, is the most aggressive form of brain tumor with poor prognosis and no cure, despite many years of efforts by clinicians and researchers to find better treatment. Biomarkers identified in GBM diagnosis, prognosis, and treatment monitoring are much less than those of other solid tumors due to the disease's biological and anatomical complexity. Currently, gliomas are diagnosed by neuroimaging, and refined diagnosis requires resection or biopsy to obtain tumor tissue for histopathological classification and tumor grading. Blood-derived biomarkers, therefore, would be very useful as minimally invasive markers that could aid in the diagnosis and management of GBM(28). The inventors here identified significantly changed genes in blood of GBM cohort. Most of the DEGs involved in the enriched pathways changed >1.4 fold in GBM (FIG. 3). Besides the reported genes in GBM, such as MMP9 and miR-221, there are newly identified genes, e.g., C1orf226 and miR-3918 in GBM, have not been reported before. The inventors also identified 12 most GBM related pathways and 20 enriched GO biological processes reflected in blood by DAVID functional analyses. The result of the network analysis shows certain miRNAs, such as miR-3918, miR-760, miR-221, miR-378g, play regulatory roles in GBMs. This data further demonstrate that removal of globin mRNAs before high-throughput RNA-seq is efficient and accurate. Therefore, the protocol the inventors used in this work has the potential to be applied in blood biomarker discovery research for a broader range of diseases.

By comprehensive analysis of the sequencing data, the inventors identified 11 gene candidates together distinguishing GBMs from non-GBM controls in current investigated patients. In the present study, the inventors have identified GBM-related molecules that are promising candidates as detective markers, many of which have high consistency in blood and tissue.

### Abbreviation

| | |
|---|---|
| GBM | Glioblastoma multiforme |
| CSF | Cerebrospinal fluid |
| PBMC | Peripheral blood mononuclear cell |
| CNS | Central nervous system |
| CTC | Circulating tumor cell |
| ctDNA/RNA | Circulating tumor DNA/RNA |
| cfDNA/RNA | Cell-free DNA/RNA |
| GR | Globin reduction |
| DGE | Differential gene expression |
| DEG | Differentially expressed gene |
| DEM | Differentially expressed miRNA |
| GO | Gene ontology |
| BP | Biological process |
| FC | Fold change |

TABLE 1
Demographic and clinical characteristics of the
patients with GBM and control for RNA-Sequencing.

| | Control | | GBM | |
|---|---|---|---|---|
| Blood for RNA-seq | | | | |
| Patient number (n) | 12 | | 10 | |
| Age in years, mean ± SD | 51.7 ± 17.2 | | 54.9 ± 11.9 | |
| Sex, male, n (%) | 7 | (58.3%) | 6 | (60%) |
| Cancer stage n (%) | N/A | | WHO-IV | (100%) |
| Recurrent, n (%) | N/A | | 6 | (60%) |
| Treatment before blood draw, n | N/A | | 8 | (80%) |
| (%) | | | | |
| Other diseases | Parkinsonism, leptomeningeal | | History of other cancer types, | |
| | disease, pancreatitis, etc. | | hypertension, diabetes, arthritis, | |
| | | | hyperlipidemia, etc. | |
| Testing Blood for RT-qPCR | | | | |
| Patient number (n) | 2 | | 5 | |
| Age in years, mean ± SD | 52.5 ± 5.5 | | 47.7 ± 17.6 | |
| Sex, male, n (%) | 1 | (50%) | 2 | (40%) |
| Cancer stage n (%) | N/A | | WHO-IV | (100%) |
| Recurrent, n (%) | N/A | | 2 | (40%) |
| Treatment before blood draw, n | N/A | | 5 | (100%) |
| (%) | | | | |
| Other diseases | Chronic migraine, palpitation, etc. | | Hyperlipidemia, hypertension, | |
| | | | osteoarthritis, etc. | |
| Tissues | | | | |
| Patient number (n) | 2 (Normal adjacent tissues of 2 | | 10 | |
| | GBM patients) | | | |
| Age in years, mean ± SD | 39 ± 20 | | 49.6 ± 13.7 | |
| Sex, male, n (%) | 2 | (100%) | 6 | (60%) |
| Cancer stage n (%) | N/A | | WHO-IV | (100%) |
| Recurrent, n (%) | N/A | | 4 | (40%) |
| Treatment before tissue | N/A | | 4 | (40%) |
| excision, n (%) | | | | |

| | | |
|---|---|---|
| Other diseases | Steroid therapy, pulmonary | Hyperlipidemia, hypertension, |
| | embolism, gastroesophageal | steroid therapy, stroke, diabetes, |
| | reflux disease, etc. | lung carcinoma, kidney cancer, etc. |

N/A, not applicable.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### E. References

The following references, to the extent that they provide exemplary procedural or other details supplementary to those set forth herein, are specifically incorporated herein by reference.
1. Glioblastoma, in Schwab M (ed): Encyclopedia of Cancer. Berlin, Heidelberg, Springer Berlin Heidelberg, 2017, pp 1911-1911
2. Hingtgen S: Glioblastoma Therapy, in Schwab M (ed): Encyclopedia of Cancer. Berlin, Heidelberg, Springer Berlin Heidelberg, 2017, pp 1911-1917
3. Muller Bark J, Kulasinghe A, Chua B, et al: Circulating biomarkers in patients with glioblastoma. Br J Cancer 122:295-305, 2020
4. Stupp R, Taillibert S, Kanner A A, et al: Maintenance Therapy With Tumor-Treating Fields Plus Temozolomide vs Temozolomide Alone for Glioblastoma: A Randomized Clinical Trial. JAMA 314:2535-43, 2015
5. Cohen M H, Johnson J R, Pazdur R: Food and Drug Administration Drug approval summary: temozolomide plus radiation therapy for the treatment of newly diagnosed glioblastoma multiforme. Clin Cancer Res 11:6767-71, 2005
6. Akers J C, Hua W, Li H, et al: A cerebrospinal fluid microRNA signature as biomarker for glioblastoma. Oncotarget 8:68769-68779, 2017
7. Figueroa J M, Skog J, Akers J, et al: Detection of wild-type EGFR amplification and EGFRvIII mutation in CSF-derived extracellular vesicles of glioblastoma patients. Neuro Oncol 19:1494-1502, 2017
8. Piccioni D E, Achrol A S, Kiedrowski L A, et al: Analysis of cell-free circulating tumor DNA in 419 patients with glioblastoma and other primary brain tumors. CNS Oncol, 2019
9. Krol I, Castro-Giner F, Maurer M, et al: Detection of circulating tumour cell clusters in human glioblastoma. Br J Cancer 119:487-491, 2018
10. Bagley S J, Nabavizadeh S A, Mays J J, et al: Clinical Utility of Plasma Cell-Free DNA in Adult Patients with Newly Diagnosed Glioblastoma: A Pilot Prospective Study. Clin Cancer Res 26:397-407, 2020
11. Nabors L B, Portnow J, Ammirati M, et al: NCCN Guidelines Insights: Central Nervous System Cancers, Version 1.2017. J Natl Compr Canc Netw 15:1331-1345, 2017
12. Wang F, Zheng Z, Guan J, et al: Identification of a panel of genes as a prognostic biomarker for glioblastoma. EBioMedicine 37:68-77, 2018
13. Mansouri A, Karamchandani J, Das S: Molecular Genetics of Secondary Glioblastoma, in De Vleeschouwer S (ed): Glioblastoma. Brisbane (AU), 2017
14. Weller M, van den Bent M, Hopkins K, et al: EANO guideline for the diagnosis and treatment of anaplastic gliomas and glioblastoma. Lancet Oncol 15:e395-403, 2014
15. Weller M, Felsberg J, Hartmann C, et al: Molecular predictors of progression-free and overall survival in patients with newly diagnosed glioblastoma: a prospective translational study of the German Glioma Network. J Clin Oncol 27:5743-50, 2009
16. Hartmann C, Hentschel B, Wick W, et al: Patients with IDH1 wild type anaplastic astrocytomas exhibit worse prognosis than IDH1-mutated glioblastomas, and IDH1 mutation status accounts for the unfavorable prognostic effect of higher age: implications for classification of gliomas. Acta Neuropathol 120:707-18, 2010
17. Christians A, Adel-Horowski A, Banan R, et al: The prognostic role of IDH mutations in homogeneously treated patients with anaplastic astrocytomas and glioblastomas. Acta Neuropathol Commun 7:156, 2019
18. Borovecki F, Lovrecic L, Zhou J, et al: Genome-wide expression profiling of human blood reveals biomarkers for Huntington's disease. Proc Natl Acad Sci USA 102:11023-8, 2005
19. Osman I, Bajorin D F, Sun T T, et al: Novel blood biomarkers of human urinary bladder cancer. Clin Cancer Res 12:3374-80, 2006
20. Martin K J, Graner E, Li Y, et al: High-sensitivity array analysis of gene expression for the early detection of disseminated breast tumor cells in peripheral blood. Proc Natl Acad Sci USA 98:2646-51, 2001
21. Wang Q, Yu Q, Lin Q, et al: Emerging salivary biomarkers by mass spectrometry. Clinica Chimica Acta 438:214-221, 2015
22. Liew C C, Ma J, Tang H C, et al: The peripheral blood transcriptome dynamically reflects system wide biology: a potential diagnostic tool. J Lab Clin Med 147:126-32, 2006
23. Ponnampalam S N, Kamaluddin N R, Zakaria Z, et al: A blood-based gene expression and signaling pathway analysis to differentiate between high and low grade gliomas.
Oncol Rep 37:10-22, 2017
24. Liebner S, Fischmann A, Rascher G, et al: Claudin-1 and claudin-5 expression and tight junction morphology are altered in blood vessels of human glioblastoma multiforme. Acta Neuropathol 100:323-31, 2000
25. Wolburg H, Wolburg-Buchholz K, Kraus J, et al: Localization of claudin-3 in tight junctions of the blood-brain barrier is selectively lost during experimental autoimmune encephalomyelitis and human glioblastoma multiforme. Acta Neuropathol 105:586-92, 2003
26. Gerstner E R, Fine R L: Increased permeability of the blood-brain barrier to chemotherapy in metastatic brain tumors: establishing a treatment paradigm. J Clin Oncol 25:2306-12, 2007
27. Ma C, Nguyen H P T, Luwor R B, et al: A comprehensive meta-analysis of circulation miRNAs in glioma as potential diagnostic biomarker. PLoS One 13:e0189452, 2018
28. Westphal M, Lamszus K: Circulating biomarkers for gliomas. Nat Rev Neurol 11:556-66, 2015
29. Xu B J, An Q A, Srinivasa Gowda S, et al: Identification of blood protein biomarkers that aid in the clinical assessment of patients with malignant glioma. Int J Oncol 40:1995-2003, 2012
30. Gallego Perez-Larraya J, Paris S, Idbaih A, et al: Diagnostic and prognostic value of preoperative combined GFAP, IGFBP-2, and YKL-40 plasma levels in patients with glioblastoma. Cancer 120:3972-80, 2014
31. Hormigo A, Gu B, Karimi S, et al: YKL-40 and matrix metalloproteinase-9 as potential serum biomarkers for patients with high-grade gliomas. Clin Cancer Res 12:5698-704, 2006
32. Dong L, Li Y, Han C, et al: miRNA microarray reveals specific expression in the peripheral blood of glioblastoma patients. Int J Oncol 45:746-56, 2014
33. Chen J, Yang L, Wang X: Reduced circulating microRNA-203 predicts poor prognosis for glioblastoma. Cancer Biomark 20:521-526, 2017
34. Regazzo G, Terrenato I, Spagnuolo M, et al: A restricted signature of serum miRNAs distinguishes glioblastoma from lower grade gliomas. J Exp Clin Cancer Res 35:124, 2016
35. Wang Z Q, Zhang M Y, Deng M L, et al: Low serum level of miR-485-3p predicts poor survival in patients with glioblastoma. PLoS One 12:e0184969, 2017
36. Zachariah M A, Oliveira-Costa J P, Carter B S, et al: Blood-based biomarkers for the diagnosis and monitoring of gliomas. Neuro Oncol 20:1155-1161, 2018
37. Kros J M, Mustafa D M, Dekker L J, et al: Circulating glioma biomarkers. Neuro Oncol 17:343-60, 2015
38. Pritchard C C, Kroh E, Wood B, et al: Blood cell origin of circulating microRNAs: a cautionary note for cancer biomarker studies. Cancer Prev Res (Phila) 5:492-497, 2012
39. Tian Z, Palmer N, Schmid P, et al: A practical platform for blood biomarker study by using global gene expression profiling of peripheral whole blood. PLoS One 4:e5157, 2009
40. Andrews S: FastQC A Quality Control tool for High Throughput Sequence Data, 2014
41. Kim D, Langmead B, Salzberg S L: HISAT: a fast spliced aligner with low memory requirements. Nat Methods 12:357-60, 2015
42. Li H, Handsaker B, Wysoker A, et al: The Sequence Alignment/Map format and SAMtools. Bioinformatics 25:2078-9, 2009
43. Liao Y, Smyth G K, Shi W: featureCounts: an efficient general purpose program for assigning sequence reads to genomic features. Bioinformatics 30:923-30, 2014
44. Rozowsky J, Kitchen R R, Park J J, et al: exceRpt: A Comprehensive Analytic Platform for Extracellular RNA Profiling. Cell Syst 8:352-357 e3, 2019
45. Anders S, Huber W: Differential expression analysis for sequence count data. Genome Biol 11:R106, 2010
46. Love M I, Huber W, Anders S: Moderated estimation of fold change and dispersion for RNA-seq data with DESeq2. Genome Biol 15:550, 2014
47. Metsalu T, Vilo J: ClustVis: a web tool for visualizing clustering of multivariate data using Principal Component Analysis and heatmap. Nucleic Acids Res 43:W566-70, 2015
48. Gaujoux R, Seoighe C: A flexible R package for nonnegative matrix factorization. BMC Bioinformatics 11:367, 2010
49. Friedman R C, Farh K K, Burge C B, et al: Most mammalian mRNAs are conserved targets of microRNAs. Genome Res 19:92-105, 2009
50. Rouillard A D, Gundersen G W, Fernandez N F, et al: The harmonizome: a collection of processed datasets gathered to serve and mine knowledge about genes and proteins. Database (Oxford) 2016, 2016
51. Huang da W, Sherman B T, Lempicki R A: Systematic and integrative analysis of large gene lists using DAVID bioinformatics resources. Nat Protoc 4:44-57, 2009
52. Shannon P, Markiel A, Ozier 0, et al: Cytoscape: a software environment for integrated models of biomolecular interaction networks. Genome Res 13:2498-504, 2003
53. Brennan C W, Verhaak R G, McKenna A, et al: The somatic genomic landscape of glioblastoma. Cell 155:462-77, 2013
54. Cancer Genome Atlas Research N: Comprehensive genomic characterization defines human glioblastoma genes and core pathways. Nature 455:1061-8, 2008
55. Harris L W, Lockstone H E, Khaitovich P, et al: Gene expression in the prefrontal cortex during adolescence: implications for the onset of schizophrenia. BMC Med Genomics 2:28, 2009
56. Berchtold N C, Cribbs D H, Coleman P D, et al: Gene expression changes in the course of normal brain aging are sexually dimorphic. Proc Natl Acad Sci USA 105:15605-10, 2008
57. Murat A, Migliavacca E, Gorlia T, et al: Stem cell-related “self-renewal” signature and high epidermal growth factor receptor expression associated with resistance to concomitant chemoradiotherapy in glioblastoma. J Clin Oncol 26:3015-24, 2008
58. Reifenberger G, Weber R G, Riehmer V, et al: Molecular characterization of long-term survivors of glioblastoma using genome- and transcriptome-wide profiling. Int J Cancer 135:1822-31, 2014
59. Sturm D, Witt H, Hovestadt V, et al: Hotspot mutations in H3F3A and IDH1 define distinct epigenetic and biological subgroups of glioblastoma. Cancer Cell 22:425-37, 2012
60. Chaussabel D, Quinn C, Shen J, et al: A modular analysis framework for blood genomics studies: application to systemic lupus erythematosus. Immunity 29:150-64, 2008
61. Chaussabel D, Baldwin N: Democratizing systems immunology with modular transcriptional repertoire analyses. Nat Rev Immunol 14:271-80, 2014
62. Livak K J, Schmittgen T D: Analysis of relative gene expression data using real-time quantitative PCR and the 2(−Delta Delta C(T)) Method. Methods 25:402-8, 2001
63. Semenza G L: Targeting HIF-1 for cancer therapy. Nat Rev Cancer 3:721-32, 2003
64. Lombardi M Y, Assem M: Glioblastoma Genomics: A Very Complicated Story, in De Vleeschouwer S (ed): Glioblastoma. Brisbane (AU), 2017
65. Welsh C T: in Ray SK (ed): Glioblastoma: Molecular Mechanisms of Pathogenesis and Current Therapeutic Strategies, Springer-Verlag New York, 2010, pp 85-94
66. Shin H, Shannon C P, Fishbane N, et al: Variation in RNA-Seq transcriptome profiles of peripheral whole blood from healthy individuals with and without globin depletion. PLoS One 9:e91041, 2014
67. Wade A, Robinson A E, Engler J R, et al: Proteoglycans and their roles in brain cancer. FEBS J 280:2399-417, 2013
68. Huang H: Matrix Metalloproteinase-9 (MMP-9) as a Cancer Biomarker and MMP-9 Biosensors: Recent Advances. Sensors (Basel) 18, 2018
69. De Palma M, Biziato D, Petrova T V: Microenvironmental regulation of tumour angiogenesis. Nat Rev Cancer 17:457-474, 2017
70. Rao J S: Molecular mechanisms of glioma invasiveness: the role of proteases. Nat Rev Cancer 3:489-501, 2003
71. Hagemann C, Anacker J, Ernestus R I, et al: A complete compilation of matrix metalloproteinase expression in human malignant gliomas. World J Clin Oncol 3:67-79, 2012
72. Lau S K, Chu P G, Weiss L M: CD163: a specific marker of macrophages in paraffin-embedded tissue samples. Am J Clin Pathol 122:794-801, 2004
73. Fabriek B O, van Bruggen R, Deng D M, et al: The macrophage scavenger receptor CD163 functions as an innate immune sensor for bacteria. Blood 113:887-92, 2009
74. Galea J, Cruickshank G, Teeling J L, et al: The intrathecal CD163-haptoglobin-hemoglobin scavenging system in subarachnoid hemorrhage. J Neurochem 121:785-92, 2012
75. Shiraishi D, Fujiwara Y, Horlad H, et al: CD163 Is Required for Protumoral Activation of Macrophages in Human and Murine Sarcoma. Cancer Res 78:3255-3266, 2018
76. Chen T, Chen J, Zhu Y, et al: CD163, a novel therapeutic target, regulates the proliferation and stemness of glioma cells via casein kinase 2. Oncogene 38:1183-1199, 2019
77. Prosniak M, Harshyne L A, Andrews D W, et al: Glioma grade is associated with the accumulation and activity of cells bearing M2 monocyte markers. Clin Cancer Res 19:3776-86, 2013
78. Martinez-Lage M, Lynch T M, Bi Y, et al: Immune landscapes associated with different glioblastoma molecular subtypes. Acta Neuropathol Commun 7:203, 2019
79. Morgan R, Feng G J, Pandha H: Abstract A193: Transmembrane protein TMEM92 as a novel target in prostate cancer. Molecular Cancer Therapeutics 12:A193-A193, 2014
80. Pei J, Grishin N V: Unexpected diversity in Shisa-like proteins suggests the importance of their roles as transmembrane adaptors. Cell Signal 24:758-69, 2012
81. Listing H, Mardin W A, Wohlfromm S, et al: MiR-23a/-24-induced gene silencing results in mesothelial cell integration of pancreatic cancer. Br J Cancer 112:131-9, 2015
82. Anastas J, Moon R: WNT signaling pathways as therapeutic targets in cancer. Nature reviews. Cancer 13:11-26, 2012
83. Van Rompay A R, Johansson M, Karlsson A: Identification of a novel human adenylate kinase. cDNA cloning, expression analysis, chromosome localization and characterization of the recombinant protein. Eur J Biochem 261:509-17, 1999
84. Solaroli N, Panayiotou C, Johansson M, et al: Identification of two active functional domains of human adenylate kinase 5. FEBS Lett 583:2872-6, 2009
85. Buttner T, Hornig C R, Busse O, et al: CSF cyclic AMP and CSF adenylate kinase in cerebral ischaemic infarction. J Neurol 233:297-303, 1986
86. Lai Y, Hu X, Chen G, et al: Down-regulation of adenylate kinase 5 in temporal lobe epilepsy patients and rat model. J Neurol Sci 366:20-26, 2016
87. Sallusto F, Lenig D, Forster R, et al: Two subsets of memory T lymphocytes with distinct homing potentials and effector functions. Nature 401:708-12, 1999
88. Mahnke Y D, Brodie™, Sallusto F, et al: The who's who of T-cell differentiation: human memory T-cell subsets. Eur J Immunol 43:2797-809, 2013
89. Mohme M, Schliffke S, Maire C L, et al: Immunophenotyping of Newly Diagnosed and Recurrent Glioblastoma Defines Distinct Immune Exhaustion Profiles in Peripheral and Tumor-infiltrating Lymphocytes. Clin Cancer Res 24:4187-4200, 2018
90. Boyle S T, Ingman W V, Poltavets V, et al: The chemokine receptor CCR7 promotes mammary tumorigenesis through amplification of stem-like cells. Oncogene 35:105-15, 2016
91. Boyle S T, Gieniec K A, Gregor C E, et al: Interplay between CCR7 and Notch1 axes promotes stemness in MMTV-PyMT mammary cancer cells. Mol Cancer 16:19, 2017
92. Rygiel T P, Karnam G, Goverse G, et al: CD200-CD200R signaling suppresses anti-tumor responses independently of CD200 expression on the tumor. Oncogene 31:2979-88, 2012
93. Coles S J, Wang E C, Man S, et al: CD200 expression suppresses natural killer cell function and directly inhibits patient anti-tumor response in acute myeloid leukemia. Leukemia 25:792-9, 2011
94. Wang C Y, Hsieh Y T, Fang K M, et al: Reduction of CD200 expression in glioma cells enhances microglia activation and tumor growth. J Neurosci Res 94:1460-1471, 2016
95. Delierneux C, Kouba S, Shanmughapriya S, et al: Mitochondrial Calcium Regulation of Redox Signaling in Cancer. Cells 9, 2020
96. Patron M, Granatiero V, Espino J, et al: MICU3 is a tissue-specific enhancer of mitochondrial calcium uptake. Cell Death Differ 26:179-195, 2019
97. Brandes N, Linial N, Linial M: Quantifying gene selection in cancer through protein functional alteration bias. Nucleic Acids Res 47:6642-6655, 2019
98. Kaushik Siddharth Manvati M, Khan J, Verma N, et al: Association of miR-760 with cancer: an overview. Gene: 144648, 2020
99. Carter J V, Galbraith N J, Yang D, et al: Blood-based microRNAs as biomarkers for the diagnosis of colorectal cancer: a systematic review and meta-analysis. Br J Cancer 116:762-774, 2017
100. Iwaya T, Fukagawa T, Suzuki Y, et al: Contrasting expression patterns of histone mRNA and microRNA 760 in patients with gastric cancer. Clin Cancer Res 19:6438-49, 2013
101. Draaisma K, Chatzipli A, Taphoorn M, et al: Molecular Evolution of IDH Wild-Type Glioblastomas Treated With Standard of Care Affects Survival and Design of Precision Medicine Trials: A Report From the EORTC 1542 Study. J Clin Oncol 38:81-99, 2020

### Example 2: Transcriptome Landscape on Patients' Whole Blood with Hemoglobin Depletion Reveals Glioblastoma Biomarkers

Biomarkers for early detection, the monitoring of disease progression, and therapy response are lacking for glioblastoma, the most common and aggressive brain tumor with a poor prognosis in adults. Development of such biomarkers for glioblastoma is critical to improving patient survival.

Here the inventors show that a unique integrated application of whole transcriptome profiling of human whole blood and globin reduction enables the identification of potential biomarker/biomarker signature for glioblastoma patients from the discovery phase.

Hemoglobin mRNA removal prior to RNA sequencing of whole blood samples improves the detection sensitivity and informative reads for differential gene expression (DGE) analysis. DGE and functional enrichment analyses of blood RNA-sequencing data revealed significantly changed genes and perturbed pathways in glioblastoma patients' blood compared with blood from non-glioblastoma controls. By performing integrated analyses, the inventors identified glioblastoma associated differentially expressed genes including coding and non-coding RNAs with at least two-fold changes. Through a further expression validation in prospective patients, 10 genes (GBM Dx Panel, GDP) were found showing significant expression changes in glioblastoma blood compared with controls and showing similar expression in a patient's blood and tumor tissue specimens.

The GDP identified as minimally invasive biomarkers have potential to be developed as a useful tool to facilitate the diagnosis and clinical management of glioblastoma.

Glioblastoma multiforme (GBM) is the most aggressive and heterogeneous type of brain cancer, aka malignant brain glioma (WHO grade IV) which occurs in the human central nervous system (CNS) with a poor survival (1,2). Despite multidisciplinary treatments such as surgery, chemotherapy, and radiotherapy, the median survival time for patients with GBM ranges in median between 12 and 15 months. The diagnosis, response monitoring of therapeutic interventions, and clinical assessment of GBM prognosis often rely on imaging techniques, and surgical brain biopsy, which is highly invasive and sometimes only partially reflect the disease status due to limited tissue pieces. Despite advanced marker discovery techniques using liquid biopsies, such as blood and cerebrospinal fluid, for the detection of circulating tumor cells (CTCs), cell-free nucleic acids and extracellular vesicles (3-7), there are still few effective GBM specific liquid biomarkers, especially blood-derived markers, available in clinical settings to reduce the necessity of multiple invasive brain biopsies and to assist the application of imaging diagnosis and surveillance. Molecular characteristics identified from GBM tumor tissue including methylation of O⁶-methylguanine-DNA methyltransferase (MGMT) gene promoter region, mutation of isocitrate dehydrogenase (IDH), alteration (such as amplification) of epidermal growth factor receptor (EGFR), and abnormalities of chromosome (for example, 1p/19q co-deletion) are the few markers currently used in GBM diagnosis and prognosis (8-14). GBM patients with MGMT methylation usually respond better to temozolomide treatment (15-17). Patients with IDH mutations appear to have a better outcome than patients with IDH wildtype; however, most primary GBMs are IDH wildtype (18-20). To help improve the survival of GBM patients, minimally invasive and ultrasensitive biomarkers or biomarker signature as indicators for disease diagnosis, therapy responses are recognized as essential tools in pressing need.

Blood is an attractive medium for the identification of disease indicators because of their critical roles in immune response, metabolism, communications with cells, and formation of extracellular matrices in various tissues and organs in the human body, and the simplicity and less invasive nature of sample collection (21-24). Serum is the liquid part of blood after coagulations, and devoid of clotting factors as fibrinogen while plasma is the liquid, cell-free part of blood, that has been treated with anti-coagulants. Blood cells include red blood cells, white blood cells, and platelets. Normally, the blood-brain barrier (BBB), composed of tightly packed cells, is highly selective on permeating molecules. However, recently, it has been found that BBBs of brain tumor patients are constantly disrupted due to loss of tight junction proteins (25-27), secreted tumorous stimuli may trigger a detectable response in gene expression of circulating blood cells (28), making it possible to detect GBM associated circulating molecules in peripheral blood (PB).

Early attempts to identify biomarkers from whole blood of cancer patients have provided little knowledge so far, one major obstacle is the existence of abundant amounts of hemoglobin in human PB. To address this translational barrier, the inventors have developed an efficient approach named as WBGR-Dx (whole blood globin reduction-diagnosis) for the identification of whole blood biomarkers. The inventors showed in previous work that globin reduction (GR) prior to high-throughput differential gene expression (DGE) analysis were able to dramatically improve the sensitivity of gender marker detection in blood (33). In this study, the inventors aim to further develop this approach and investigate gene expression differences in whole blood samples comparing GBMs and non-GBM controls by the combination strategy of GR with RNA-sequencing (RNA-seq).

### A. Methods

### 1. Patients and Experiment Design

In this study, a total of 15 GBM patients and 14 non-GBM control individuals were recruited for blood sample collection at Baylor Scott & White Medical Center, Temple, Texas, USA. Clinical and demographic details of all participants have been summarized in Table 1. In addition, ten tumor specimens from GBM patients and two normal adjacent tissues were obtained from Baylor Scott & White Neuroscience Institute Brain Bank. Clinical information of tissue samples used in this study is also included in Table 1. This study was approved by Baylor Scott & White Health Institutional Review Board and was performed in compliance with local and federal requirements for research involving human subjects. Written informed consent forms were obtained from the participants or their proxies. The overall experimental design is depicted in FIG. 1.

### 2. RNA Preparation

For whole blood specimens, PB from participants was drawn with a BD safety-Lok™ blood collection set (BD) into PAXgene™ RNA collection tube (Qiagen) and then kept at −80° C. Total RNAs including small RNAs were isolated with the PAXgene™ Blood miRNA Kit (Qiagen). For brain tissue specimens, total RNAs including small RNAs were extracted using miRNeasy Mini Kit (Qiagen). The quantity of isolated RNAs was determined using NanoDrop Spectrophotometer (NanoDrop Technologies). The quality of RNAs was evaluated using the 2100 Bioanalyzer system (Aligent).

### 3. Globin Reduction (GR)

The GLOBINclear™ Kit (Ambion) was employed to remove the highly abundant hemoglobin mRNAs from the blood isolated RNA samples. Briefly, 1 μg total RNAs from each sample were hybridized with a biotinylated Capture OLIGO Mix which is specific for human mRNA hemoglobin a and b. Streptavidin Magnetic Beads were added to bind the biotinylated oligonucleotides that hybridized with globin mRNAs and the remaining RNAs were then pulled down by magnet. The globin depleted RNAs were further purified with a rapid magnetic bead-based purification process. The resulting samples were stored at −80° C. before use.

4. Library Construction and RNA-Sequencing for mRNAs

RNA samples both pre- and post-GR with an RNA integrity number (RIN)≥7.0 were used for cDNA paired-end library construction. First, mRNAs and RNAs with poly (A) tail (such as some long noncoding RNAs) were purified from total RNA using oligo (dT)-attached magnetic beads and were then fragmented. First-strand cDNAs were generated using random hexamer-primed reverse transcription, followed by a second-strand cDNA synthesis. Then PCR was performed and PCR products were purified with AMPure XP beads (Agencount, Beckman Coulter) and library quality was validated on the 2100 Bioanalyzer system (Agilent). The double-stranded PCR products were heat-denatured and circularized by the splint oligo sequence. The single-strand circle DNA (ssCir DNA) was formatted as the final library. The libraries were sequenced on an Illumina Hiseq2000 platform at Beth Israel Deaconess Medical Center (BIDMC) Genomics Proteomics Core in Harvard Medical School and 100 bp paired-end reads were generated.

### 5. Small RNA Library Construction and Sequencing

Small RNA library construction and sequencing were performed at BIDMC Genomics Proteomics Core. Briefly, approximately 1 μg RNA with a RIN≥7.0 from each sample was used. RNA segments of different sizes were separated by PAGE gel, 18-30 nt stripes were selected and recycled. Then 3′ adaptor connection system, RT primer addition and 5′ adaptor connection system were prepared. After that, strand cDNA synthesis, PCR amplification and library fragment selection were performed. The double-stranded PCR products were heat denatured and circularized by the splint oligo sequence. The single-strand circle DNA (ssCir DNA) was formatted as the final library. The libraries were validated on the 2100 Bioanalyzer system (Agilent). Then the libraries were sequenced on a BGISEQ-500 platform (Beijing Genomics Institute, China) at BIDMC Genomics Proteomics Core in Harvard Medical School.

### 6. Quality Control and Bioinformatic Analyses of RNA-Seq Data

mRNA-Sequencing (mRNA-Seq) generated an average of ˜87 million raw reads for each sample. After quality filtering by removing reads containing adapters, Poly-N, or have low quality from the total raw reads, an average of 43.7 million high-quality clean reads were obtained for each sample. The small RNA-seq yielded an average of 34.27 million raw reads for each sample, and 23.05 million (67%) clean reads were retained. The quality characteristics of sequence reads for each sample is over 97%. Q20 for each sample is over 97%, GC contents are around 49% to 59% for sequenced samples. Quality control for raw sequencing reads was performed by the FastQC software (34). Reads from mRNA-seq were aligned to the human reference genome (GRCh38) using hisat2 (35). 97.6% of the clean reads from mRNA-seq and 89% of the clean reads from small RNA-seq were mapped to the human genome. SAM files obtained from the aligner were converted to BAM format using SAMtools (36). FeatureCounts (37) was used to quantify the total number of counts for each gene. The small RNA sequencing reads were aligned and counted using the extra-cellular RNA processing toolkit (exceRpt) (38). Briefly, the pipeline first filtered the reads that mapped to UniVec vector and ribosomal RNA sequences and the unmapped reads were then aligned to the human genome (hg38) and quantified for different types of RNAs including miRNAs (miRBase v21) and other small RNAs.

### 7. DGE Analysis and Data Visualization

Both mRNA-Seq and small RNA-Seq read counts were normalized using the median-of-ratios method (39) and logarithmic base 2 (log 2) transformed for data visualization. DGE analysis was performed with DESeq2 package in R (40); genes that survived FDR 0.05 or P-value ≤0.05 were considered differentially expressed. Gene expression heatmaps were plotted using the ClustVis webtool (41) or the NMF package in R (42). Hierarchical clustering of representative mRNA and miRNA expressions were performed to reveal reproducibility in biological replicates.

### 8. DGE Analysis of GBM Tissue Datasets and Data Visualization

The Cancer Genome Atlas (TCGA) (11) database was used to download the transcriptome RNA profiling data for 156 primary GBM tissues and 5 solid normal tissue controls (13). DGE analysis was performed with DESeq2 package in R (40); genes that survived FDR 0.05 were considered differentially expressed. Heatmaps were plotted using the NMF package in R (42). The generated DEG list was compared with blood RNA-seq data to identify overlapped genes. R2: Genomics Analysis and Visualization Platform was also used to analyze gene expression differences of GBM tissues compared to normal brain tissues. Two public datasets of normal brain tissues (“Normal Brain PFC-Harris-44-MAS5.0-u133p2” (43) and “Normal Brain regions-Berchtold-172-MAS5.0 u133p2” (44)) and three public datasets of GBM tumor tissues (“Tumor Glioblastoma-Hegi-84MAS5.0-u133p2” (45), Tumor Glioblastoma-Loeffler-70-MAS5.0-u133p2” (46) and “Tumor Glioblastoma-Pfister-46-MAS5.0-u133p2” (47)) were selected and analyzed through R2: megasearch online portal. Gene expression differences were considered significant at FDR≤0.01. The generated DEGs were also compared with blood RNA-seq data to identify overlapped genes. DEGs from tissue RNA-seq data and blood RNA-seq data were used for functional analysis, which was performed with Reactome Pathway Database (48). Annotated reactome pathways with FDR (q-value)≤0.1 were considered significantly enriched and were shown by dot plot generated using ggplot2 package (49) in R. The network showing major pathways included in both blood data and tissue data was visualized by figure generated using networkD3 package (50) in R. Analysis and visualization of accuracy, sensitivity and specificity of the gene candidates was performed by ROCit package (51) in R.

### 9. Modular Gene Enrichment Analysis

To facilitate interpretation of the gene expression signature, the inventors used a pre-existing framework of 260 transcriptional modules included more than 14000 transcripts specific to blood samples collected from a wide range of diseases, established by Chaussabel et al., to analyze this dataset to enrich immune related genes associated with GBM (52,53). For each module, the percentage of transcripts significantly up- or down-regulated was calculated, and the module score was defined as the difference in percent up or down, designated as the proportion. If a module in which x % transcripts are significantly up-regulated and y % transcripts are significantly down-regulated, the module score would be x−y. Proportion values of x−y>0 or x−y<0 are represented by a red or blue spot, respectively. Data was considered significant at FDR≤0.05 and visualized using the R package. This approach can detect small but co-dependent changes in transcripts that may not be considered to be significant when analyzing each gene as an independent variable (52).

### 10. Reverse Transcription-Quantitative Polymerase Chain Reaction (RT-qPCR) Analysis

Total RNAs including small RNAs were extracted from tumor tissues and blood of GBM patients or control counterparts using miRNeasy Mini Kit (Qiagen) and PAXgene™ Blood miRNA Kit (Qiagen), respectively. For miRNA detection, total RNAs were used to complete reverse transcription and PCR with the miScript PCR system including miScript II RT Kit, miScript Primer Assays and miScript SYBR Green PCR Kit (Qiagen). For mRNA detection, total RNAs were used to prepare cDNA using iScript Reverse Transcription Supermix (Bio-Rad), and then subjected to qPCR assay. RT-qPCR was performed using iTaq Universal SYBR Green Supermix (Bio-Rad). All the qPCR assays were performed in triplicate experiments on CFX96 Touch™ Real-Time PCR Detection System. Cycle threshold (Ct) values were calculated using the automated settings of the system. Fold change (FC) obtained from Ct values using 2^{-ΔΔCt} methodology (54) was converted into log 2 for statistical analysis. Human GAPDH, U6 and 18S rRNA were used as controls for PCRs of mRNAs, miRNAs and long non-coding RNAs (lncRNAs), respectively. Gene specific primers were designed using Primer-BLAST online tool. The primer sequences are listed in Supplemental Table S2. Data were presented as mean±standard deviation (SD).

### 11. Statistical Analysis

The level of significance for gene expression difference between GBM and non-GBM control groups was analyzed by the moderated t-statistic in datasets analysis for RNA-seq data using the DESeq2 package in R or student t-test for RT-qPCR data using R package. Statistical significance was defined as FDR≤0.05 or P-value ≤0.05 according to the analysis.

### B. Results

1. Globin mRNA Reduction Improves Informative Reads of Blood RNA-Seq

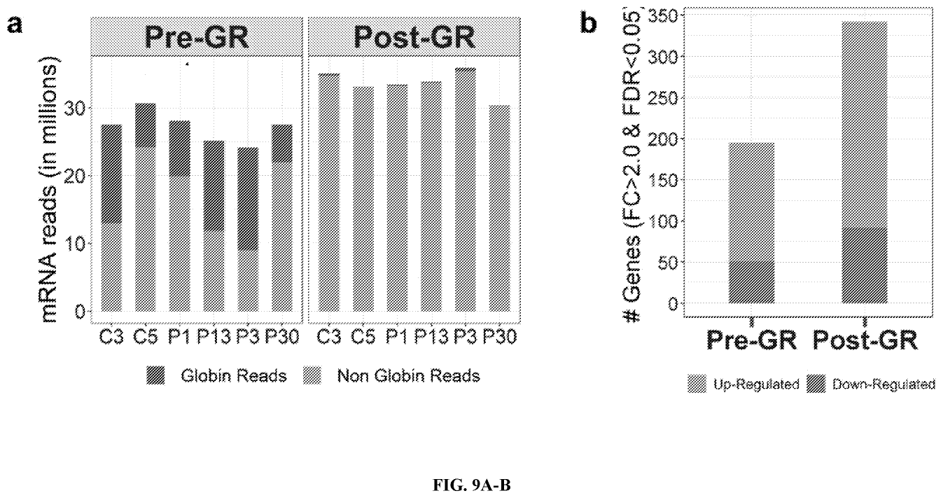

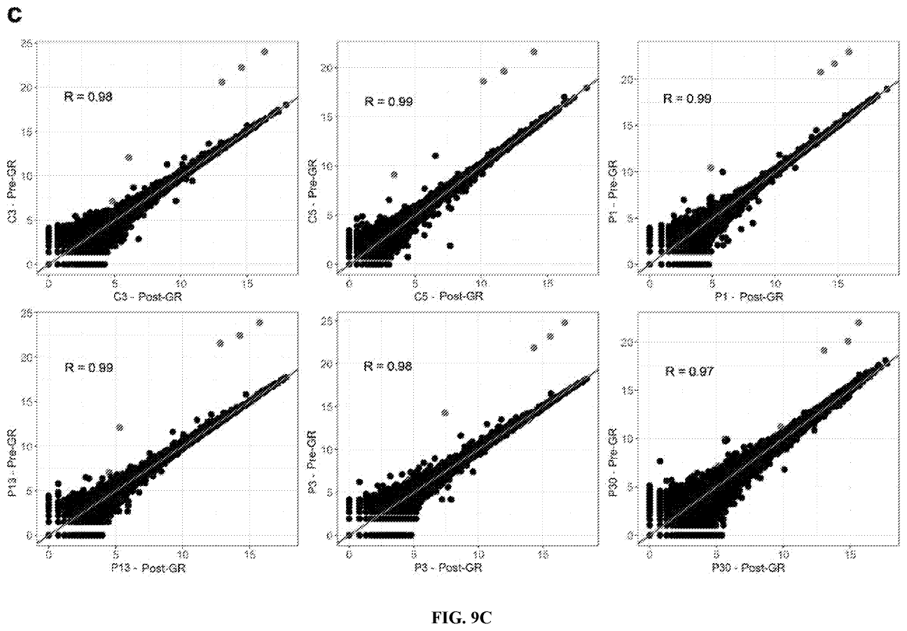

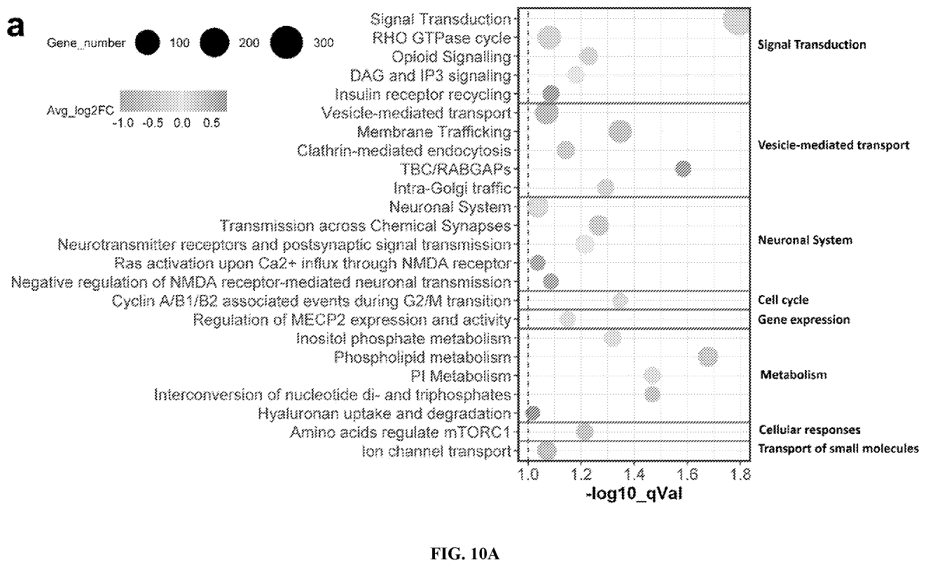

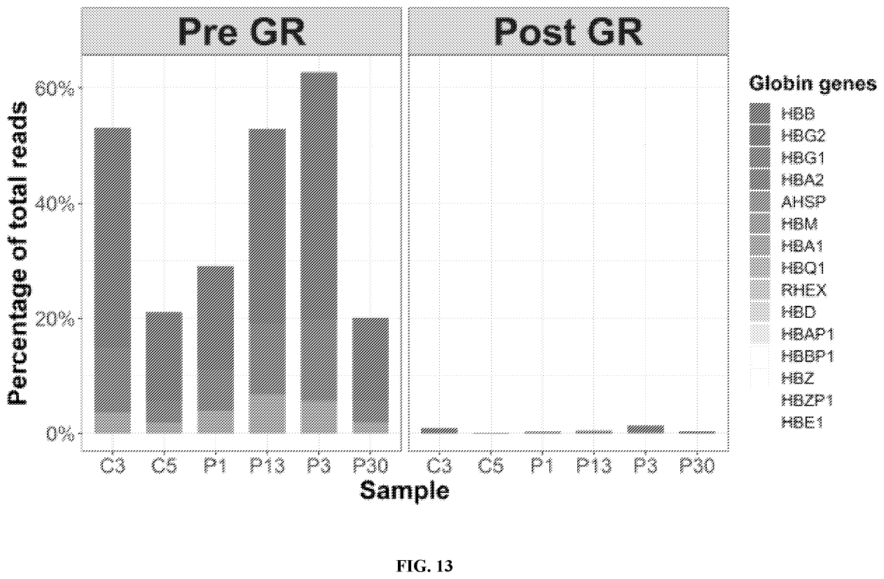

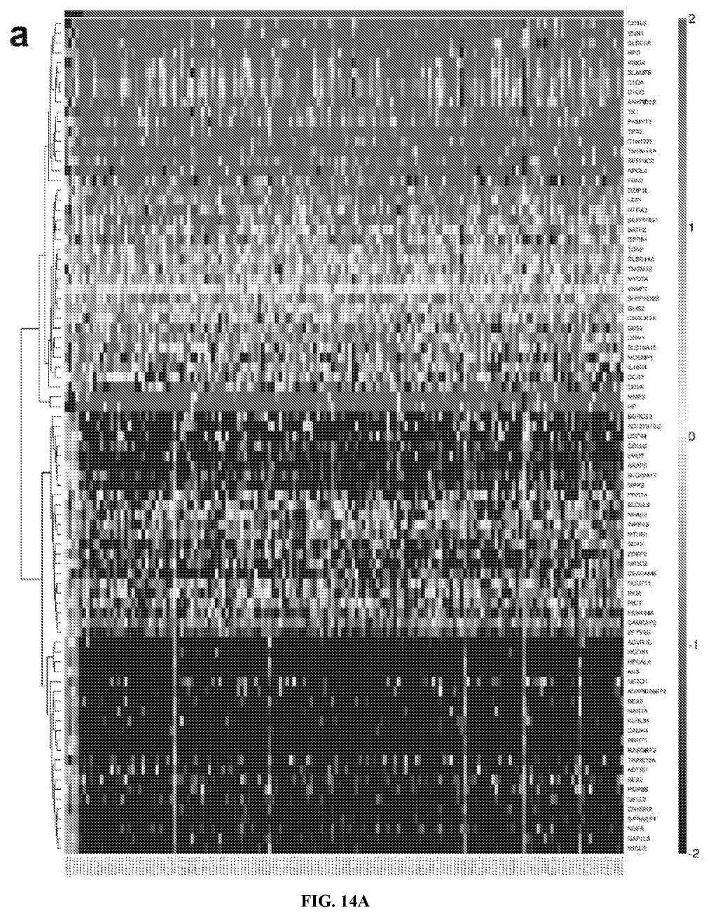

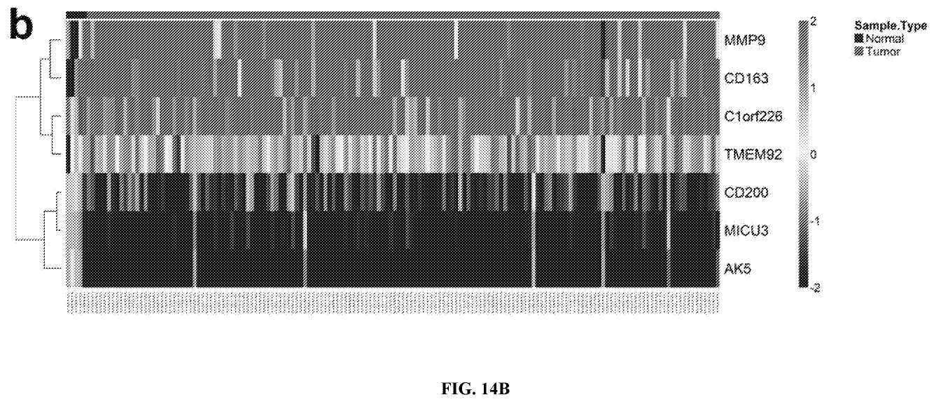

To identify GBM-associated gene expression signature through the comparison between non-GBM individuals and GBM patients by the means of deep RNA-seq, whole blood samples from 12 non-GBM controls and 10 GBM patients were analyzed. The clinical demographic characteristics are shown in Table 1. Total RNAs including small RNAs from the collected whole blood samples were isolated. Then, globin mRNA depletion for the isolated blood RNAs were performed followed by both mRNA-seq and small RNA-seq. To assess the impact of GR on RNA-seq results, half of the prepared RNAs from four GBM patients (designated as P1, P3, P13 and P30) and two control individuals (designated as C3 and C5) were left as pre-GR controls and the remaining half of the RNAs were subjected to globin mRNA removal. The reduction of globin mRNA reads was first analyzed and confirmed in the sequencing data (FIG. 13). As shown in FIG. 9a, GR reduced the percentage of total reads dominated by globin genes from 20-63% per sample to less than 1.5%. Meanwhile, GR increased the number of non-globin mRNA reads by an average of over two folds for both upregulated and downregulated genes (FIG. 9b). In addition, DGE analysis identified more than 31.5% GBM-related DEGs (FDR≤0.05) in post-GR samples than pre-GR samples. Concordance of gene expression levels pre-GR and post-GR in a given sample was analyzed by Pearson correlation and visualized using scatter plots. The high correlation coefficient of concordance with R≥0.97 (FIG. 9c) indicates that GR didn't introduce bias to the expression detection of non-globin genes. Collectively, the inventors here demonstrate that GR increases the detection sensitivity and improves the informative reads from blood RNA-seq.

### 2. DGE Analysis of RNA-Seq Data Reveals Significantly Changed Genes in GBM Blood

To analyze the gene expression profiling differences between control and GBM groups, sequencing data of all post-GR samples were analyzed. DGE analysis was then performed. Comparisons were performed with the adjustment for gender. Changes of mRNAs with FDR≤0.05 and miRNAs with P-value ≤0.05 were considered significant, i.e., differentially expressed. Differentially expressed genes (DEGs) and differentially expressed miRNAs (DEMs) with FC≥2.0 were selected and used for further analysis and data visualization. As a result, 487 genes (250 up, 237 down) survived the cutoff of FC≥2.0 and FDR≤0.05, and 30 miRNAs (7 up, 23 down) survived the cutoff of FC≥2.0 and P-value ≤0.05.

### 3. DGE Analysis of TCGA and R2 Tissue Databases Reveals Circulating Genes in GBM Blood

To assess the correlation of gene expression in whole blood and tissue samples, the inventors accessed and analyzed the public available transcriptome profiling data of GBM tumor tissues. The Cancer Genome Atlas (TCGA) (11) database was accessed to download RNA-Seq data of 156 primary GBM tissues and 5 solid normal tissue controls. DGE analysis comparing GBM tumor tissues with normal tissue controls revealed a total of 9314 DEGs (FC>2.0 and FDR≤0.05) in GBM tissue data. Additionally, overlapped genes were found in the DEGs identified from GBM tissues and the DEGs identified from GBM blood. The inventors also queried R2 Genomics Analysis and Visualization Platform (r2.amc.nl) to identify potential DEGs in R2 GBM tissue datasets. Two normal brain tissue datasets and three GBM tumor tissue datasets were comparatively analyzed using the R2 online tool. A total of 542 DEGs (FC>2.0 and FDR≤0.01) were identified. Comparison of blood DEGs identified from RNA-seq and tissue DEGs identified from TCGA and R2 database was then performed, and 73 overlapped DEGs in tissue and blood with FC over 2.0 and FDR<0.01 were obtained. This result indicates that gene expression in whole blood partially reflects GBM tissue features, which reveals the existence of circulating molecules in GBM blood and strongly supports the possibility of blood biomarker identification for brain tumors like GBM.

### 4. Functional Enrichment Analysis Reveals Perturbed Signaling Pathways Reflected in GBM Blood

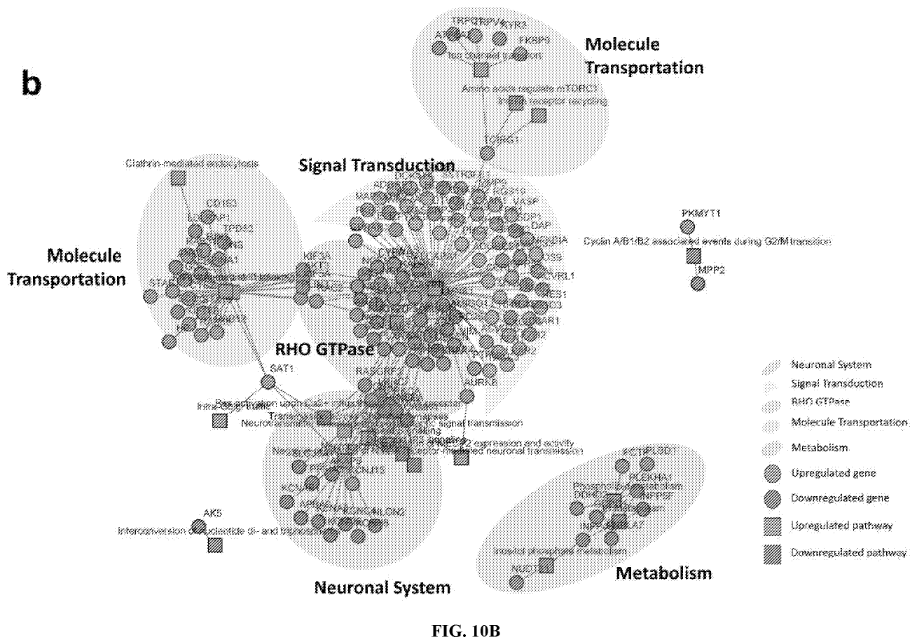

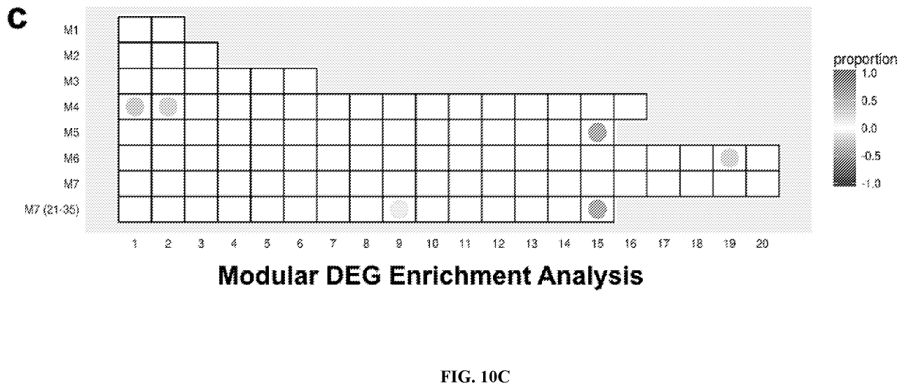

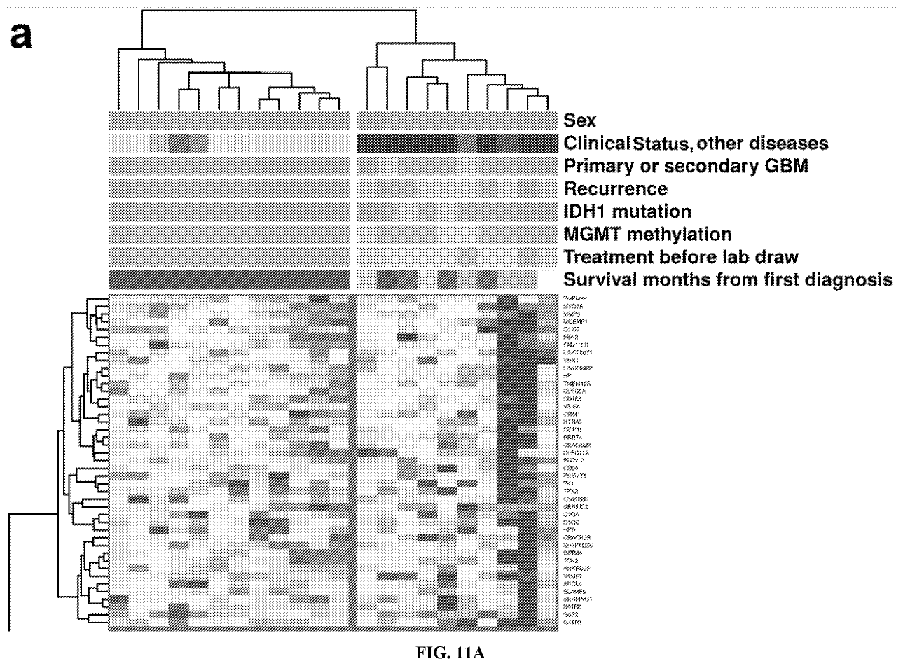

To further characterize the main biological functions and key pathways involving the DEGs identified from GBM blood and tissue transcriptome profiling data, functional enrichment was performed via Reactome pathway platform and enriched signaling pathways were selected using a threshold of FDR<0.1 and FC>1.4 (considering that significant change may not occur for every participant in a cellular signaling). A previous study analyzing GBM tumor tissues revealed that genes involved in RTK (Receptor Tyrosine Kinases) signaling, PI3K (Phosphoinositide 3-kinase) signaling, MAPK (Mitogen-activated protein kinase) signaling, P53 and RB1 (retinoblastoma gene) signaling pathways were found frequently mutated in GBM tumorous cells and therefore dysfunction of these pathways maybe largely associated with GBM (11). Comparison of perturbed signaling pathways between blood and tissue data revealed that several sub-signaling pathways involved in signal transduction, neuronal system, cell cycle, transport of small molecules, metabolism, and so on are reflected in GBM blood (FIG. 10a). Gene network connecting DEGs involved in these pathways was shown in FIG. 10b. These observations suggest that some common tumor-related alterations of signaling pathways can be detected in blood, supporting the application of whole blood samples for biomarker development for GBMs.

### 5. Modular Analysis Facilitates DEG Enrichment

From a previous blood gene modular analysis, gene sets that changed in a coordinated manner have been clustered into modules to facilitate blood transcriptome analyses (52). Next, to enrich DEGs related to the immune system in GBM blood, these module repertories were used to seek for significantly changed immune-related gene sets from the RNA-seq data of GBM blood. The DEGs identified with FC≥2.0 were subjected to module enrichment analysis. Module number shows selection rounds of transcripts, modules are more specific as the number goes higher. For example, module M1 is generic and may change in many diseases (52). As shown in FIG. 10c, differences were observed at modules M4.1, M4.2, M5.15, M6.19, M7.29 and M7.35, genes in which were selected for further analysis. Among those modules, M4.1 showed genes involved in T cell function, M4.2 showed genes involved in inflammation and M5.15 showed genes linked to Neutrophils.

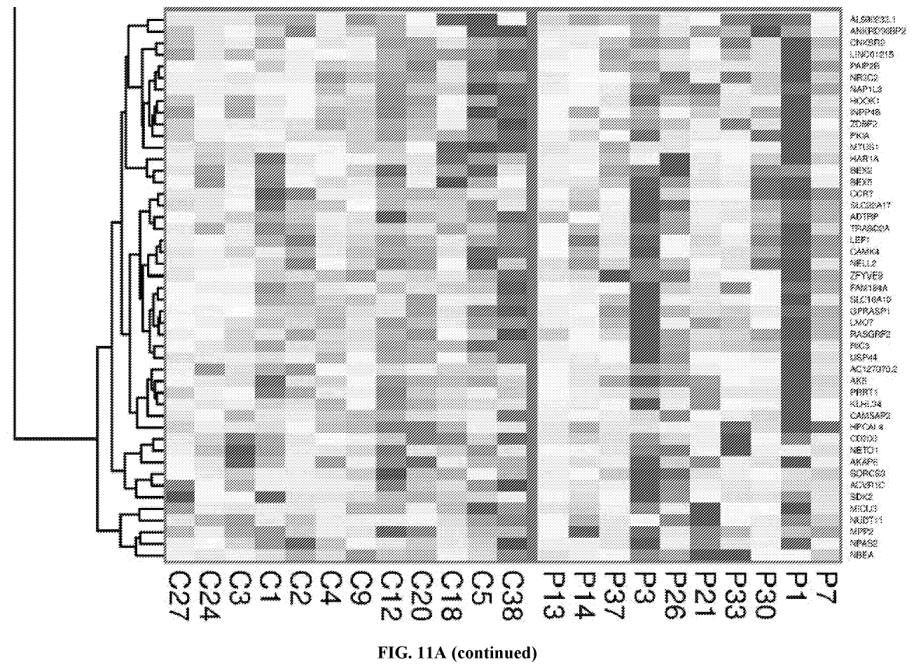

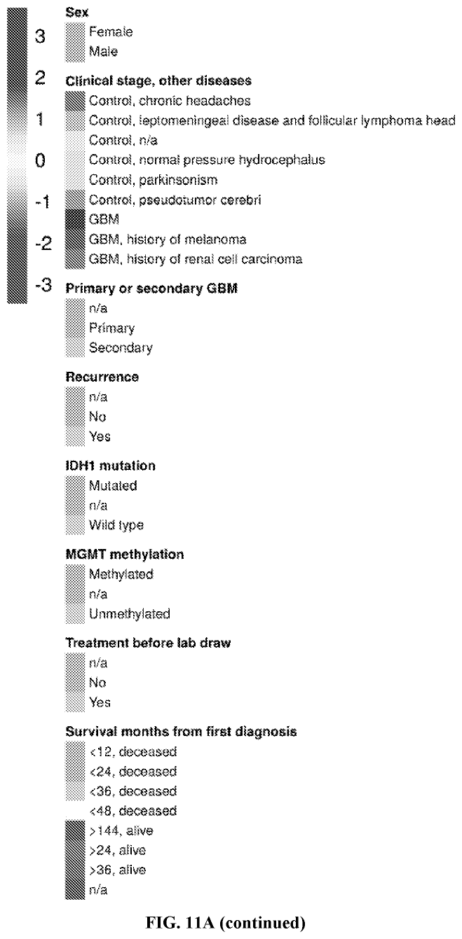

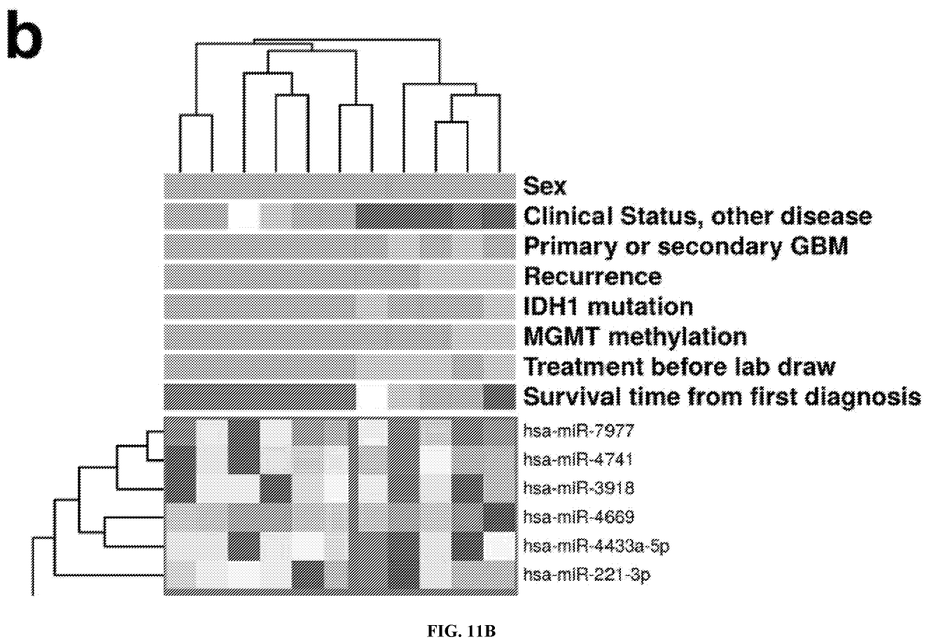

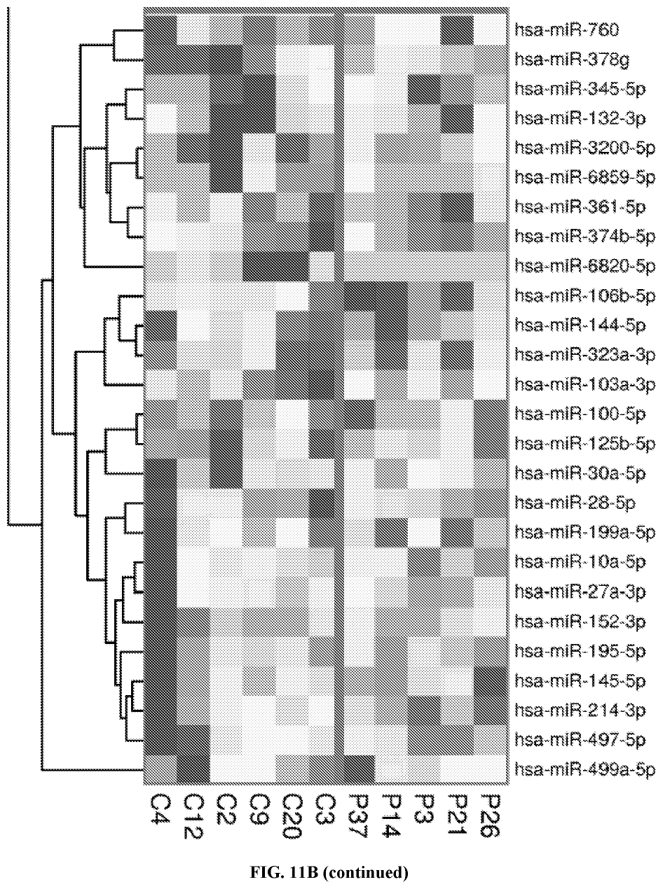

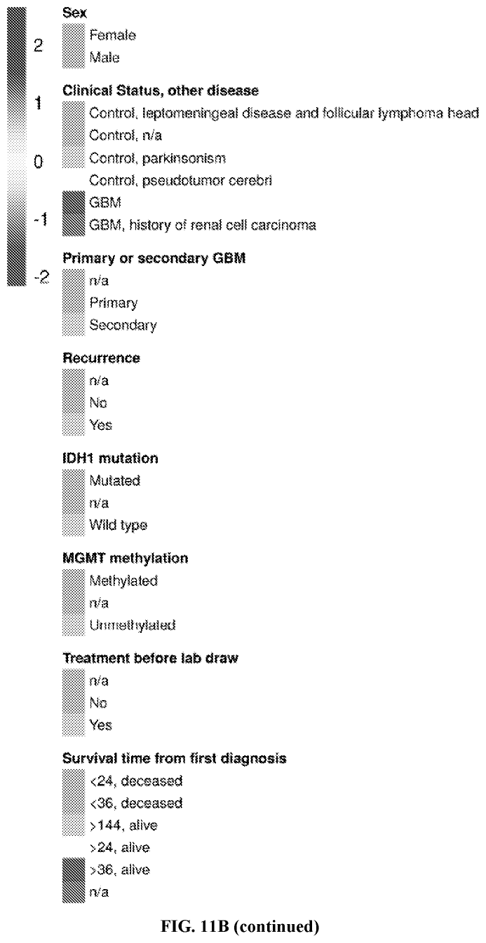

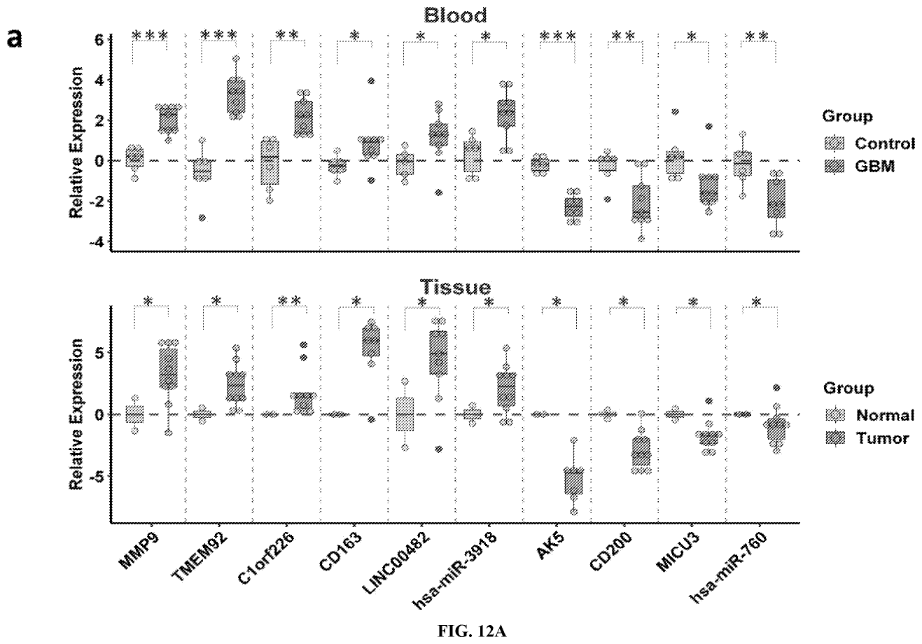

Collectively, after the integrated analyses of GBM tissue RNA-seq data from public databases, and Baylor modules, 90 DEGs were further enriched and summarized with patient clinical characteristics in a heatmap shown in FIG. 11a. Meanwhile, an expression heatmap of the 30 DEMs identified from blood RNA-seq was shown in FIG. 11b.

### 6. Gene Expression Validation in Prospective Independent Whole Blood and Tissue Specimens Elicits a Panel of GBM Related Blood Marker Candidates

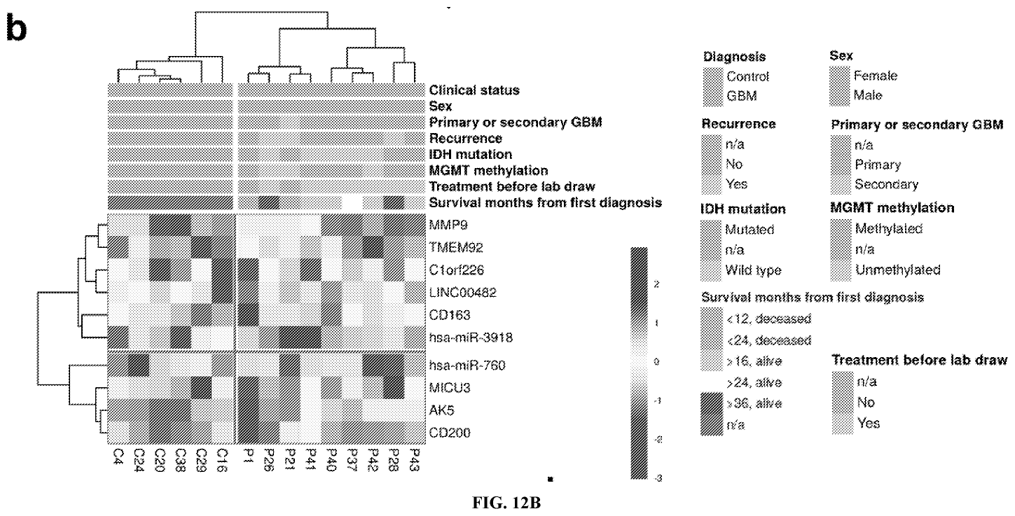

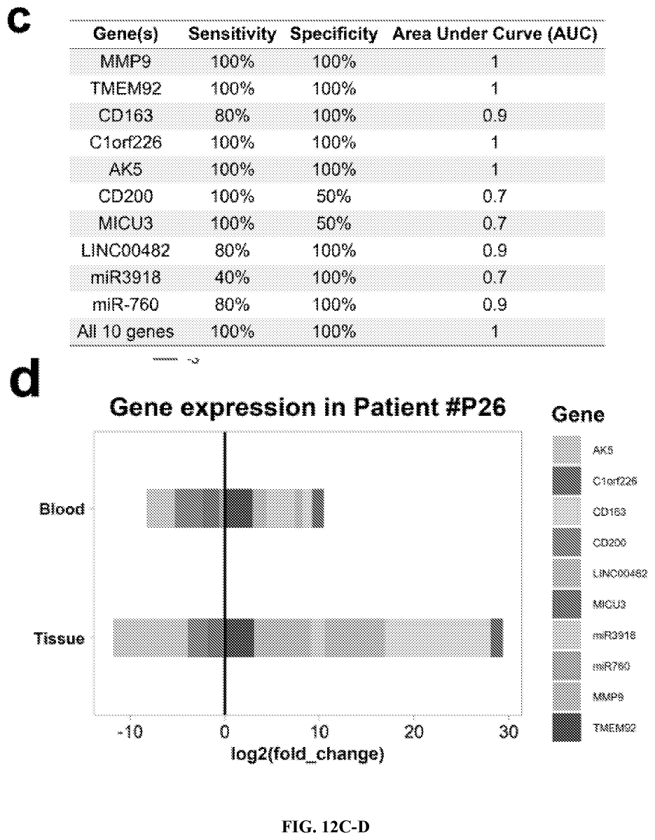

To further identify GBM-specific genes/miRNAs from the afore-described analyses, whole blood samples from two healthy individuals (designated as C16 and C29) and five GBM patients (designated as P28, P40, P41, P42, and P43) were further collected and were used as independent testing samples. The inventors selected nine DEGs and two DEMs to perform RT-qPCR experiments. PCR results were normalized by the mean of the control group and then log 2 transformed for visualization. Blood samples that have been used in RNA-seq including controls C4, C20, C24 and C38 and GBMs P1, P21, P26, and P37 were used as methodological positive controls. For miRNAs, C24, C38 and P1, which were not used in miRNA-seq, were also considered as testing samples in miRNA validation. From the result, the gene expression pattern of these samples in RT-qPCR data was consistent with RNA-seq data, which validated the reliability of RT-qPCR used in this work. The PCR results were further summarized in FIG. 12a upper panel using boxplots. To evaluate the expression of these genes in GBM tumor cells, 10 tumor tissues and two normal adjacent tissues from GBM patients were used to perform qPCR. The results were summarized in FIG. 12a lower panel using boxplots.

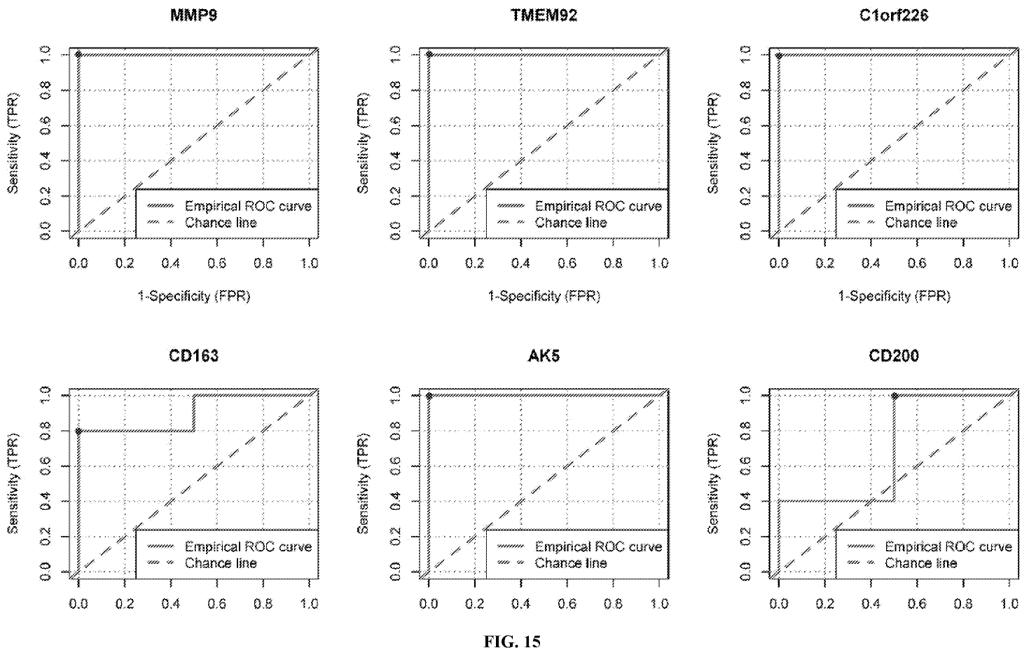

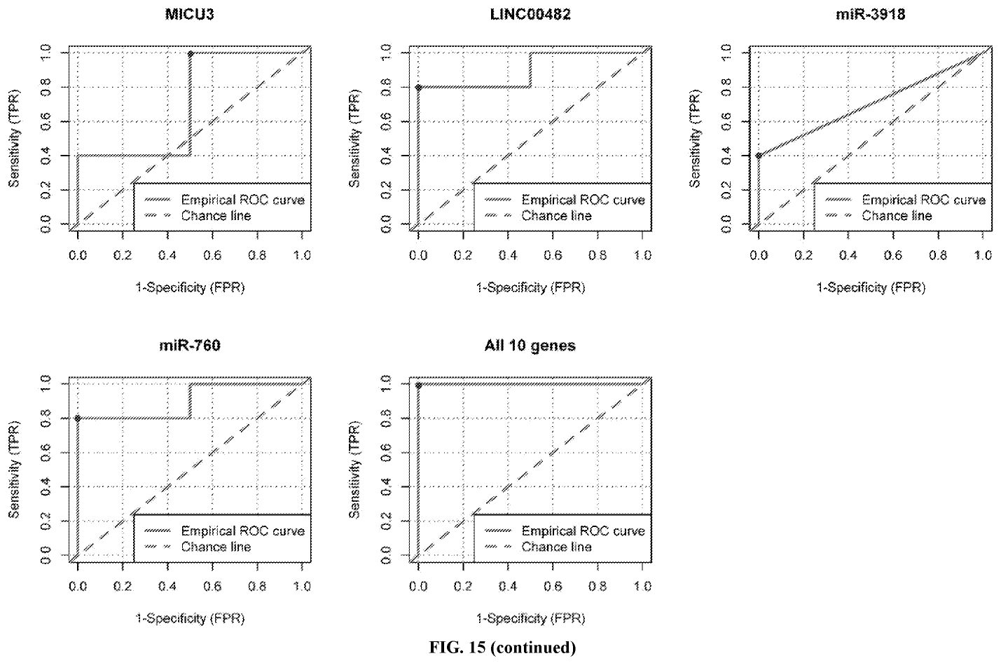

PCR data of GBM blood were also visualized using heatmap with clinical information (FIG. 12b). Additionally, the inventors trained a logistic regression model using the selected DEGs on 22 GBM blood RNA-Seq samples (12 controls, 10 GBMs) and validated the trained model on the seven independent blood samples (2 controls, 5 GBM) and achieved 100% accuracy to classify GBM samples. Sensitivities, specificities and values of area under ROC (receiver operating characteristic) curve (AUC) for each individual gene or all 10 genes combined together were further analyzed. The results were summarized in FIG. 12c and FIG. 15.

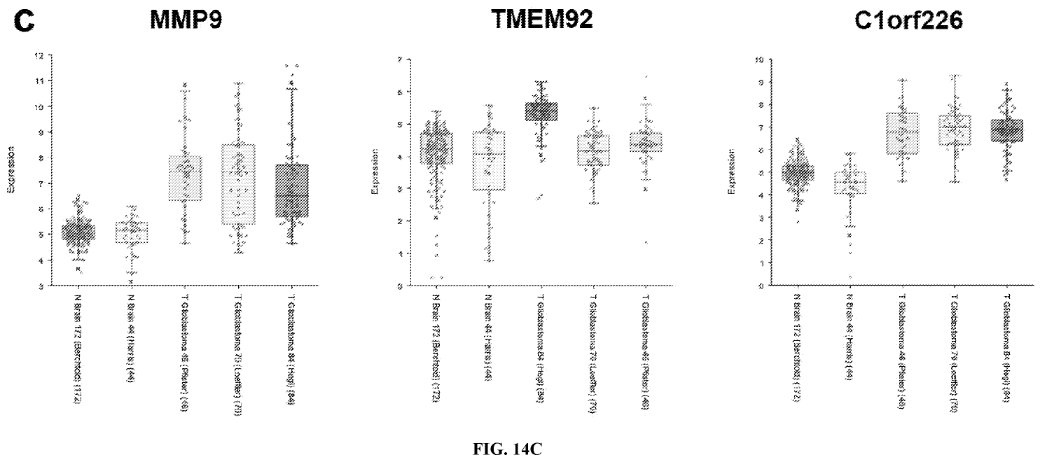

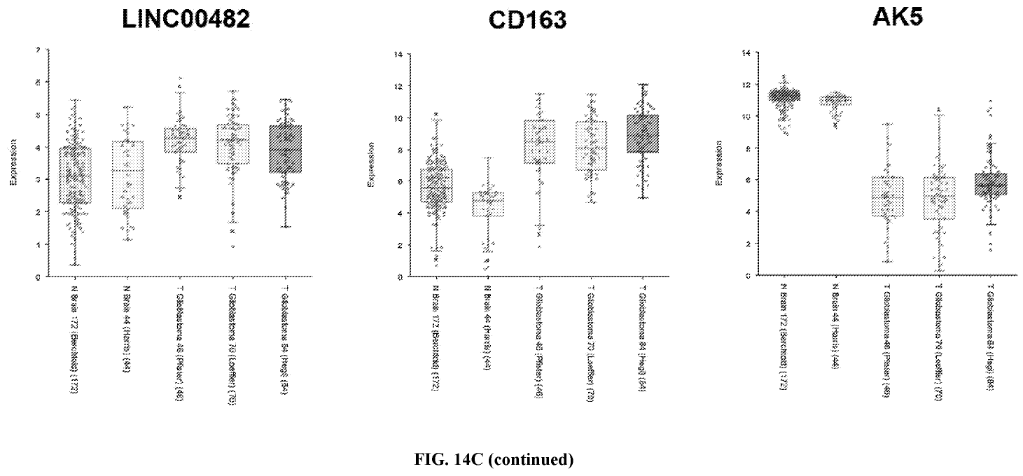

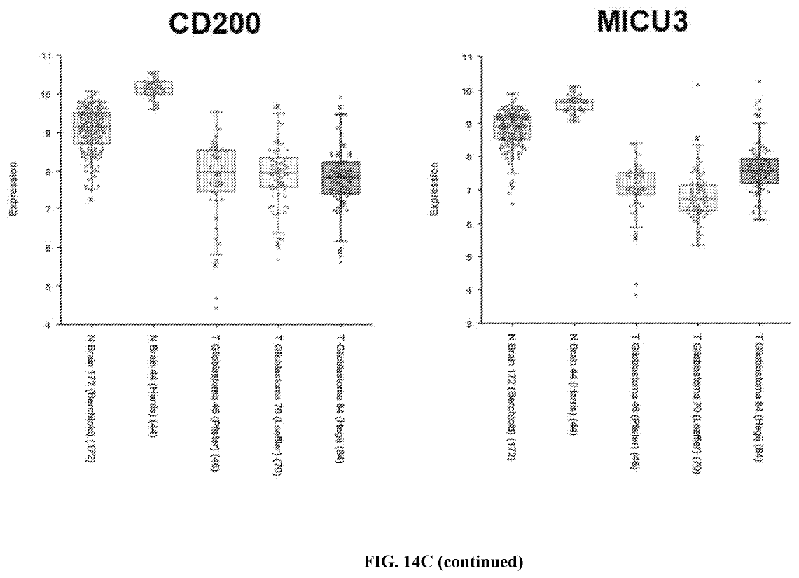

Expression levels of the 10 candidate genes were also analyzed using TCGA GBM RNA-seq data and R2 Genomics database in GBM cohorts. Results were summarized in FIG. 14, which showed significant expression difference of MMP9, C1orf226, CD163, LINC00482, AK5, MICU3 and CD200 in tumor tissue data (miRNA expression data are not available in TCGA and R2 GBM tissue datasets). TCGA data of the all 90 enriched genes in blood RNA-seq were also analyzed and showed in FIG. 14. Furthermore, in the investigated patient cohort, the inventors have both tumor tissue and blood sample available for one patient, the inventors thus analyzed the expressions of the 10 genes, and found that all 10 genes found from the blood sample reflected their expression in the matched tumor tissue (FIG. 12d). Taken together, 10 potential GBM blood markers are identified including the expression signature of 7 genes, 1 lncRNA and 2 miRNAs.

### C. Discussion

In this study, the inventors discover that the combination strategy of transcriptome profiling of patients' whole blood with GR named as WBGR-Dx (whole blood globin reduction-diagnosis) has the capability to identify a gene expression signature in GBM blood. In an early study, the inventors report that GR improves useful biological signals (33). They showed that there is a clear performance improvement when finding gender marker genes using post-globin reduction samples instead of pre-globin reduction samples after blood profiling. Here they further show that the GR has the benefit of increasing the informative reads of RNA-seq and detective sensitivity of DGE using WBGR-Dx for GBM. In total, they identified 487 DEGs with FC≥2.0 and FDR≤0.05, and 30 DEMs with FC≥2.0 and P-value ≤0.05 from RNA-seq after the blood GR. Reactome pathway enrichment analysis of the DEGs from GBM TCGA tissue data reveals that signal transduction, neuronal system, metabolism, etc. are perturbed and are reflected in GBM blood with GR. Moreover, through a merging analysis of tissue data from TCGA and R2, and Baylor module gene enrichment analysis, a final 90 DEGs were elicited. By RT-qPCR, 10 blood marker candidates including 7 mRNAs, 1 lncRNA, and 2 miRNAs were validated in additional newly collected whole blood samples and tissue specimens. Specifically, MMP9, TMEM92, C1orf226, CD163, LINC00482 and miR-3918 are upregulated, AK5, CD200, MICU3, and miR-760 are downregulated in GBM. Among them, genes such as MMP9 and CD163 are known players in GBM development. Successful identification of these genes validated the approach to identify novel genes such as C1orf226, TMEM92, AK5, MICU3, and miR-3918 that may play equally important roles in GBM. Furthermore, differential expressions of these genes were found in both blood and matched tissue samples in a GBM patient, although this does not exclude the possibility that several genes, if not all, identified here are changed simultaneously in tumor tissue and blood samples of other GBM patients considering the highly heterogeneous nature of this disease.

Currently, GBM is diagnosed by neuroimaging, and refined diagnosis requires invasive brain biopsy to obtain tumor tissue for histopathological classification and tumor grading. Blood-derived biomarkers, therefore, would be very useful as minimally invasive markers that could aid in the diagnosis and clinical management of GBM (30). In RNA-seq, to improve the accurate detection and to increase the sequencing capacity of informative RNA reads, GR is considered as a critical step for blood RNA-seq from previous and current studies. Abundant globin RNAs will increase the amount of noise for subsequent sequencing after an amplification step of RNA samples. Also, during sequencing, a high amount of globin reads could reduce the reads of non-globin RNAs. Therefore, hemoglobin mRNAs removal prior to sequencing library preparation significantly improves the overall informative reads of the sequencing data and hence has resulted in the detection of 63% more DEGs from this study (FIG. 10a). With the improvement of technique, the inventors here identified significantly changed genes in the blood of the GBM cohort. They also identified the most GBM related pathways reflected in blood by Reatome pathway analysis. Therefore, the protocol used in this work can be applied in blood biomarker discovery research for a broader range of diseases, e.g., other types of cancer.

By comprehensive analysis of the sequencing data, the inventors identified 10 gene candidates (GBM Dx Panel, GDP) together distinguishing GBMs from non-GBM controls in current investigated patients. The inventors observed CD163 higher expression in >60% of GBM blood samples and in >80% GBM tumor tissues (FIG. 11a and 12). These results prove the important role of CD163 and MMP9 as potential markers and therapeutic targets for GBM. It was found that a low expression of CD200 is observed and shows a high coherence in both GBM blood and tumor tissue compared to control.

From these results, there is a variety of DEGs that have not been reported to be associated with GBM before and their functions are largely unknown, such as LINC00482, TMEM92, C1orf226, miR-3918 and miR-760. The elevated level of LINC00482 in the GBM group may be a novel potential detectable biomarker and therapeutic target. According to the analysis of TCGA and R2 genomics databases, higher expression of C1orf226 in tumor tissue is observed in GBM cohorts. The results shown in FIGS. 11 and 12 also suggest C1orf226 overexpression as a feature of most GBMs in both blood and tissue samples. The inventors first found that a high level of miR-3918 is associated with GBM which may be a potential blood biomarker. The combo of TMEM92 and miR-3918 expression may be used as both GBM biomarker and therapeutic target. The inventors show that miR-760 expression decreases in GBM patients and a lower level of miR-760 may also serve as a GBM indicator.

In the present study, the inventors have identified GBM associated molecules that are promising candidates as detective markers, many of which have high consistency in blood and tissue. Meanwhile, there are three aspects that the inventors want to discuss. First, some blood samples are from patients after treatments, which may be one of the reasons for intragroup gene expression variation. The inclusion of more GBM blood samples before treatments will strengthen the power of the identified marker candidates. Second, the status of IDH mutation, MGMT methylation or EGFR amplification is not available for all patients, the inclusion of these pieces of information in future study would allow a correlation analysis of the identified gene signature with these known GBM characteristics. Third, DGE analysis comparing newly diagnosed GBMs and recurrent GBMs was not a main focus of this study. Considering a high recurrence rate of GBM, specific works distinguishing disease progression will be helpful for GBM prognosis and treatment response assessment. Nevertheless, the differential expression of the current proposed GDP is likely a common trait of GBM blood. A prospective cohort would be warranted for further justification of the identified marker candidates from this work and for the achievement of high specificity and sensitivity and contribute to translate these findings towards clinical use as a robust GBM classifier.

### D. Abbreviations

AK5: Adenylate kinase 5; BBB: Blood-brain barrier; C1orf226: Chromosome 1 open reading frame 226; CD14: Cluster of differentiation 14; CD68: Cluster of differentiation 68; CD163: Cluster of differentiation 163; CD200: Cluster of differentiation 200; CD204: Cluster of differentiation 204; CD200R: CD200 receptor; CNS: Central nervous system; CSF: Cerebrospinal fluid; CTC: Circulating tumor cell; DEG: Differentially expressed gene; DEM: Differentially expressed miRNA; DGE: Differential gene expression; EGFR: Epidermal growth factor receptor; FC: Fold change; FDR: False discovery rate; GAPDH: Glyceraldehyde-3-phosphate dehydrogenase; GBM: Glioblastoma multiforme; GDP: GBM Dx Panel; GR: Globin reduction; IDH: Isocitrate dehydrogenase; LINC00482: Long intergenic noncoding RNA 482; MGMT: 06-methylguanine-DNA methyltransferase; MICU3: mitochondrial calcium uptake family member 3; miRNA: microRNA; MMP9: Matrix metallopeptidase 9; MRI: Magnetic resonance imaging; mRNA: messenger RNA; PB: Peripheral blood; PBMC: Peripheral blood mononuclear cell; RNA-seq: RNA-sequencing; RT-qPCR: Reverse transcription quantitative PCR; TCGA: The Cancer Genome Atlas; TMEM92: Transmembrane Protein 92; TMZ: Temozolomide; TTF: Tumor treating fields; WBGR-Dx: whole blood globin reduction-diagnosis.

### E. Tables

TABLE 1
Demographics and clinical characteristics of the patients with GBM and control for RNA-Sequencing.

| | Control | | GBM | |
|---|---|---|---|---|
| Blood for RNA-seq | | | | |
| Patient number (n) | 12 | | 10 | |
| Age in years, mean ± SD | 51.7 ± 17.2 | | 54.9 ± 11.9 | |
| Sex, male, n (%) | 7 | (58.3%) | 6 | (60%) |
| Cancer stage n (%) | N/A | | WHO-IV | (100%) |
| Recurrent, n (%) | N/A | | 6 | (60%) |
| Treatment before blood draw, n (%) | N/A | | 8 | (80%) |
| Other diseases | Parkinsonism, leptomeningeal | | History of other cancer types, | |
| | disease, pancreatitis, etc. | | hypertension, diabetes, arthritis, | |
| | | | hyperlipidemia, etc. | |
| Testing Blood for RT-qPCR | | | | |
| Patient number (n) | 2 | | 5 | |
| Age in years, mean ± SD | 52.5 ± 5.5 | | 47.7 ± 17.6 | |
| Sex, male, n (%) | 1 | (50%) | 2 | (40%) |
| Cancer stage n (%) | N/A | | WHO-IV | (100%) |
| Recurrent, n (%) | N/A | | 2 | (40%) |
| Treatment before blood draw, n (%) | N/A | | 5 | (100%) |
| Other diseases | Chronic migraine, palpitation, | | Hyperlipidemia, hypertension, | |
| | etc. | | osteoarthritis, etc. | |
| Tissues | | | | |
| Patient number (n) | 2 | | 10 | |
| | (Normal adjacent tissues of 2 | | | |
| | GBM patients) | | | |
| Age in years, mean ± SD | 39 ± 20 | | 49.6 ± 13.7 | |
| Sex, male, n (%) | 2 | (100%) | 6 | (60%) |
| Cancer stage n (%) | N/A | | WHO-IV | (100%) |
| Recurrent, n (%) | N/A | | 4 | (40%) |
| Treatment before tissue excision, | N/A | | 4 | (40%) |
| n (%) | | | | |

| | | |
|---|---|---|
| Other diseases | Steroid therapy, pulmonary | Hyperlipidemia, hypertension, steroid |
| | embolism, gastroesophageal | therapy, stroke, diabetes, lung |
| | reflux disease, etc. | carcinoma, kidney cancer, etc. |

N/A, not applicable.

Supplemental Table S2. Primers used for real-time RT-PCR.

| | | | SEQ |
|---|---|---|---|
| | | Product | ID |
| Gene | Primer Sequences | Size | NO: |
| GADPH | Forward 5′-GAGTCAACGGATTTGGTCGT-3′ | 238 bp | 1 |
| | Reverse 5′-TTGATTTTGGAGGGATCTCG-3′ | | |
| MMP9 | Forward 5′-TTGACAGCGACAAGAAGTGG-3′ | 179 bp | 2 |
| | Reverse 5′-GCCATTCACGTCGTCCTTAT-3′ | | |
| TMEM92 | Forward 5′-AGCCAAATGTGGTCTCATCC-3′ | 119 bp | 3 |
| | Reverse 5′-CCAGGAAGATGATGACGAAGA-3′ | | |
| C1orf226 | Forward 5′-GCAGGAGGTGACACTCTCAA-3′ | 89 bp | 4 |
| | Reverse 5′-CGTGGTCAACTGTCCGAGAA-3′ | | |
| CD163 | Forward 5′-CTGGCGTGACATGTTCTGAT-3′ | 98 bp | 5 |
| | Reverse 5′-CAGTCTCTGAATCTCCACCTCAAC-3′ | | |
| AK5 | Forward 5′-GCTGCTCCATTGGTTAAATACTTCC-3′ | 108 bp | 6 |
| | Reverse 5′-GTTGTCAACTGCCATGCTGATG-3′ | | |
| CD200 | Forward 5′-CCTAAGAATCAGGTGGGGAAGGA-3′ | 137 bp | 7 |
| | Reverse 5′-GACGAGAAGAATTACCAGGGAAACA-3′ | | |
| MICU3 | Forward 5′-ACCATCAGTGAAGAAGATTTTGCTC-3′ | 114 bp | 8 |
| | Reverse 5′-TGTGATGCCCTTTTCTTCAGGT-3′ | | |
| 18S rRNA | Forward 5′-GGCCCTGTAATTGGAATGAGTC-3′ | 147 bp | 9 |
| | Reverse 5′-CCCAAGATCCAACTACGAGCTTT-3′ | | |
| LINC00482 | Forward 5′-CTCTGTGGGAGCCTAGATGG-3′ | 135 bp | 10 |
| | Reverse 5′-CCATAGCCCTTCTTAACGCC-3′ | | |
| U6 | Forward: QIAGEN PCR Control Hs_RNU6-2_1 Primer | N/A | N/A |
| | Reverse: QIAGEN Universal Primer | | |
| hsa-miR-3918 | Forward 5′-CACGAAACAGGGCCGCAG-3′ | N/A | N/A |
| | Reverse: QIAGEN Universal Primer | | |
| hsa-miR-760 | Forward 5′-CCCGGCTCTGGGTCTGTG-3′ | N/A | N/A |
| | Reverse: QIAGEN Universal Primer | | |

### F. References

1. Muller Bark, J., Kulasinghe, A., Chua, B., Day, B. W. & Punyadeera, C. Circulating biomarkers in patients with glioblastoma. Br. J. Cancer 122, 295-305, doi:10.1038/s41416-019-0603-6 (2020).
2. Hingtgen, S. in Encyclopedia of Cancer (ed Manfred Schwab) 1911-1917 (Springer Berlin Heidelberg, 2017).
3. Akers, J. C. et al. A cerebrospinal fluid microRNA signature as biomarker for glioblastoma. Oncotarget 8, 68769-68779, doi:10.18632/oncotarget.18332 (2017).
4. Figueroa, J. M. et al. Detection of wild-type EGFR amplification and EGFRvIII mutation in CSF-derived extracellular vesicles of glioblastoma patients. Neuro Oncol. 19, 1494-1502, doi:10.1093/neuonc/nox085 (2017).
5. Piccioni, D. E. et al. Analysis of cell-free circulating tumor DNA in 419 patients with glioblastoma and other primary brain tumors. CNS Oncol, doi:10.2217/cns-2018-0015 (2019).
6. Krol, I. et al. Detection of circulating tumour cell clusters in human glioblastoma. Br J Cancer 119, 487-491, doi:10.1038/s41416-018-0186-7 (2018).
7. Bagley, S. J. et al. Clinical Utility of Plasma Cell-Free DNA in Adult Patients with Newly Diagnosed Glioblastoma: A Pilot Prospective Study. Clin Cancer Res 26, 397-407, doi:10.1158/1078-0432.CCR-19-2533 (2020).
8. Nabors, L. B. et al. NCCN Guidelines Insights: Central Nervous System Cancers,
Version 1.2017. J. Natl. Compr. Canc. Netw. 15, 1331-1345, doi:10.6004/jnccn.2017.0166 (2017).
9. Wang, F. et al. Identification of a panel of genes as a prognostic biomarker for glioblastoma. EBioMedicine 37, 68-77, doi:10.1016/j.ebiom.2018.10.024 (2018).
10. Mansouri, A., Karamchandani, J. & Das, S. in Glioblastoma (ed S. De Vleeschouwer)
(2017).
11. Brennan, C. W. et al. The somatic genomic landscape of glioblastoma. Cell 155, 462-477, doi:10.1016/j.cell.2013.09.034 (2013).
12. Fujisawa, H. et al. Loss of heterozygosity on chromosome 10 is more extensive in primary (de novo) than in secondary glioblastomas. Lab. Invest. 80, 65-72, doi:10.1038/labinvest.3780009 (2000).
13. Cancer Genome Atlas Research, N. Comprehensive genomic characterization defines human glioblastoma genes and core pathways. Nature 455, 1061-1068, doi:10.1038/nature07385 (2008).
14. Wen, P. Y. & Kesari, S. Malignant gliomas in adults. N. Engl. J. Med. 359, 492-507 (2008).
15. Weller, M. et al. EANO guideline for the diagnosis and treatment of anaplastic gliomas and glioblastoma. Lancet Oncol. 15, e395-403, doi:10.1016/S1470-2045(14)70011-7 (2014).
16. Hegi, M. E. et al. Correlation of 06-methylguanine methyltransferase (MGMT) promoter methylation with clinical outcomes in glioblastoma and clinical strategies to modulate MGMT activity. J. Clin. Oncol. 26, 4189-4199, doi:10.1200/JCO.2007.11.5964 (2008).
17. Metellus, P. et al. Prognostic impact of 06-methylguanine-DNA methyltransferase silencing in patients with recurrent glioblastoma multiforme who undergo surgery and carmustine wafer implantation: a prospective patient cohort. Cancer 115, 4783-4794, doi:10.1002/cncr.24546 (2009).
18. Weller, M. et al. Molecular predictors of progression-free and overall survival in patients with newly diagnosed glioblastoma: a prospective translational study of the German Glioma Network. J. Clin. Oncol. 27, 5743-5750, doi:10.1200/JCO.2009.23.0805 (2009).
19. Hartmann, C. et al. Patients with IDH1 wild type anaplastic astrocytomas exhibit worse prognosis than IDH1-mutated glioblastomas, and IDH1 mutation status accounts for the unfavorable prognostic effect of higher age: implications for classification of gliomas. Acta Neuropathol. 120, 707-718, doi:10.1007/s00401-010-0781-z (2010).
20. Christians, A. et al. The prognostic role of IDH mutations in homogeneously treated patients with anaplastic astrocytomas and glioblastomas. Acta Neuropathol Commun 7, 156, doi:10.1186/s40478-019-0817-0 (2019).
21. Borovecki, F. et al. Genome-wide expression profiling of human blood reveals biomarkers for Huntington's disease. Proc. Natl. Acad. Sci. U.S.A. 102, 11023-11028, doi:10.1073/pnas.0504921102 (2005).
22. Osman, I. et al. Novel blood biomarkers of human urinary bladder cancer. Clin. Cancer Res. 12, 3374-3380, doi:10.1158/1078-0432.CCR-05-2081 (2006).
23. Martin, K. J. et al. High-sensitivity array analysis of gene expression for the early detection of disseminated breast tumor cells in peripheral blood. Proceedings of the National Academy of Sciences of the United States of America 98, 2646-2651, doi:10.1073/pnas.041622398 (2001).
24. Wang, Q., Yu, Q., Lin, Q. & Duan, Y. Emerging salivary biomarkers by mass spectrometry. Clin. Chim. Acta 438, 214-221, dx.doi.org/10.1016/j.cca.2014.08.037 (2015).
25. Liebner, S. et al. Claudin-1 and claudin-5 expression and tight junction morphology are altered in blood vessels of human glioblastoma multiforme. Acta Neuropathol 100, 323-331 (2000).
26. Wolburg, H. et al. Localization of claudin-3 in tight junctions of the blood-brain barrier is selectively lost during experimental autoimmune encephalomyelitis and human glioblastoma multiforme. Acta Neuropathol 105, 586-592, doi:10.1007/s00401-003-0688-z (2003).
27. Gerstner, E. R. & Fine, R. L. Increased permeability of the blood-brain barrier to chemotherapy in metastatic brain tumors: establishing a treatment paradigm. J. Clin. Oncol.
25, 2306-2312, doi:10.1200/JCO.2006.10.0677 (2007).
28. Ponnampalam, S. N. et al. A blood-based gene expression and signaling pathway analysis to differentiate between high and low grade gliomas. Oncol Rep 37, 10-22, doi:10.3892/or.2016.5285 (2017).
29. Kros, J. M. et al. Circulating glioma biomarkers. Neuro Oncol. 17, 343-360, doi:10.1093/neuonc/nou207 (2015).
30. Westphal, M. & Lamszus, K. Circulating biomarkers for gliomas. Nat. Rev. Neurol. 11, 556-566, doi:10.1038/nrneurol.2015.171 (2015).
31. Muller, C. et al. Hematogenous dissemination of glioblastoma multiforme. Sci Transl Med 6, 247ra101, doi:10.1126/scitranslmed.3009095 (2014).
32. Pritchard, C. C. et al. Blood cell origin of circulating microRNAs: a cautionary note for cancer biomarker studies. Cancer Prev. Res. (Phila.) 5, 492-497, doi:10.1158/1940-6207.CAPR-11-0370 (2012).
33. Tian, Z. et al. A practical platform for blood biomarker study by using global gene expression profiling of peripheral whole blood. PLoS One 4, e5157, doi:10.1371/journal.pone.0005157 (2009).
34. Andrews, S. FastQC A Quality Control tool for High Throughput Sequence Data.
(2014).
35. Kim, D., Langmead, B. & Salzberg, S. L. HISAT: a fast spliced aligner with low memory requirements. Nat Methods 12, 357-360, doi:10.1038/nmeth.3317 (2015).
36. Li, H. et al. The Sequence Alignment/Map format and SAMtools. Bioinformatics 25, 2078-2079, doi:10.1093/bioinformatics/btp352 (2009).
37. Liao, Y., Smyth, G. K. & Shi, W. featureCounts: an efficient general purpose program for assigning sequence reads to genomic features. Bioinformatics 30, 923-930, doi:10.1093/bioinformatics/btt656 (2014).
38. Rozowsky, J. et al. exceRpt: A Comprehensive Analytic Platform for Extracellular RNA Profiling. Cell Syst 8, 352-357 e353, doi:10.1016/j.cels.2019.03.004 (2019).
39. Anders, S. & Huber, W. Differential expression analysis for sequence count data.
Genome Biol. 11, R106, doi:10.1186/gb-2010-11-10-r106 (2010).
40. Love, M. I., Huber, W. & Anders, S. Moderated estimation of fold change and dispersion for RNA-seq data with DESeq2. Genome Biol. 15, 550, doi:10.1186/s13059-014-0550-8 (2014).
41. Metsalu, T. & Vilo, J. ClustVis: a web tool for visualizing clustering of multivariate data using Principal Component Analysis and heatmap. Nucleic Acids Res. 43, W566-570, doi:10.1093/nar/gkv468 (2015).
42. Gaujoux, R. & Seoighe, C. A flexible R package for nonnegative matrix factorization. BMC Bioinformatics 11, 367, doi:10.1186/1471-2105-11-367 (2010).
43. Harris, L. W. et al. Gene expression in the prefrontal cortex during adolescence: implications for the onset of schizophrenia. BMC Med. Genomics 2, 28, doi:10.1186/1755-8794-2-28 (2009).
44. Berchtold, N. C. et al. Gene expression changes in the course of normal brain aging are sexually dimorphic. Proc. Natl. Acad. Sci. U.S.A. 105, 15605-15610, doi:10.1073/pnas.0806883105 (2008).
45. Murat, A. et al. Stem cell-related “self-renewal” signature and high epidermal growth factor receptor expression associated with resistance to concomitant chemoradiotherapy in glioblastoma. J. Clin. Oncol. 26, 3015-3024, doi:10.1200/JCO.2007.15.7164 (2008).
46. Reifenberger, G. et al. Molecular characterization of long-term survivors of glioblastoma using genome- and transcriptome-wide profiling. Int. J. Cancer 135, 1822-1831, doi:10.1002/ijc.28836 (2014).
47. Sturm, D. et al. Hotspot mutations in H3F3A and IDH1 define distinct epigenetic and biological subgroups of glioblastoma. Cancer Cell 22, 425-437, doi:10.1016/j.ccr.2012.08.024 (2012).
48. Jassal, B. et al. The reactome pathway knowledgebase. Nucleic Acids Res. 48, D498-D503, doi:10.1093/nar/gkz1031 (2020).
49. Wickham, H. ggplot2: Elegant Graphics for Data Analysis. (Springer-Verlag New York, 2016).
50. Allaire, J. J., Gandrud, C., Russell, K. & Yetman, C. J. networkD3: D3 javascript network graphs from r. R package version 0.4 (2017).
51. Khan, M. R. A. & Brandenburger, T. ROCit: Performance Assessment of Binary Classifier with Visualization. R package version (2019).
52. Chaussabel, D. & Baldwin, N. Democratizing systems immunology with modular transcriptional repertoire analyses. Nat. Rev. Immunol. 14, 271-280, doi:10.1038/nri3642 (2014).
53. Chaussabel, D. et al. A modular analysis framework for blood genomics studies: application to systemic lupus erythematosus. Immunity 29, 150-164, doi:10.1016/j.immuni.2008.05.012 (2008).
54. Livak, K. J. & Schmittgen, T. D. Analysis of relative gene expression data using real-time quantitative PCR and the 2(−Delta Delta C(T)) Method. Methods 25, 402-408, doi:10.1006/meth.2001.1262 (2001).
55. Shin, H. et al. Variation in RNA-Seq transcriptome profiles of peripheral whole blood from healthy individuals with and without globin depletion. PLoS One 9, e91041, doi:10.1371/journal.pone.0091041 (2014).
56. Hagemann, C., Anacker, J., Ernestus, R. I. & Vince, G. H. A complete compilation of matrix metalloproteinase expression in human malignant gliomas. World J. Clin. Oncol. 3, 67-79, doi:10.5306/wjco.v3.i5.67 (2012).
57. Hormigo, A. et al. YKL-40 and matrix metalloproteinase-9 as potential serum biomarkers for patients with high-grade gliomas. Clin. Cancer Res. 12, 5698-5704, doi:10.1158/1078-0432.CCR-06-0181 (2006).
58. Chen, T. et al. CD163, a novel therapeutic target, regulates the proliferation and stemness of glioma cells via casein kinase 2. Oncogene 38, 1183-1199, doi:10.1038/s41388-018-0515-6 (2019).
59. Prosniak, M. et al. Glioma grade is associated with the accumulation and activity of cells bearing M2 monocyte markers. Clin. Cancer Res. 19, 3776-3786, doi:10.1158/1078-0432.CCR-12-1940 (2013).
60. Martinez-Lage, M. et al. Immune landscapes associated with different glioblastoma molecular subtypes. Acta Neuropathol Commun 7, 203, doi:10.1186/s40478-019-0803-6 (2019).
61. Coles, S. J. et al. CD200 expression suppresses natural killer cell function and directly inhibits patient anti-tumor response in acute myeloid leukemia. Leukemia 25, 792-799, doi:10.1038/leu.2011.1 (2011).
62. Wang, C. Y., Hsieh, Y. T., Fang, K. M., Yang, C. S. & Tzeng, S. F. Reduction of CD200 expression in glioma cells enhances microglia activation and tumor growth. J. Neurosci. Res. 94, 1460-1471, doi:10.1002/jnr.23922 (2016).
63. Friedman, R. C., Farh, K. K., Burge, C. B. & Bartel, D. P. Most mammalian mRNAs are conserved targets of microRNAs. Genome Res. 19, 92-105, doi:10.1101/gr.082701.108 (2009).
64. Rouillard, A. D. et al. The harmonizome: a collection of processed datasets gathered to serve and mine knowledge about genes and proteins. Database (Oxford) 2016, doi:10.1093/database/baw 100 (2016).
65. Kaushik Siddharth Manvati, M., Khan, J., Verma, N. & Dhar, P. K. Association of miR-760 with cancer: an overview. Gene, 144648, doi:10.1016/j.gene.2020.144648 (2020).
66. Carter, J. V. et al. Blood-based microRNAs as biomarkers for the diagnosis of colorectal cancer: a systematic review and meta-analysis. Br. J. Cancer 116, 762-774, doi:10.1038/bjc.2017.12 (2017).
67. Iwaya, T. et al. Contrasting expression patterns of histone mRNA and microRNA 760 in patients with gastric cancer. Clin. Cancer Res. 19, 6438-6449, doi:10.1158/1078-0432.CCR-12-3186 (2013).

What is claimed:

1. A method for treating a subject with glioblastoma multiforme (GBM), the method comprising treating the subject for GBM after the expression level of one or more biomarkers selected from MMP9, TMEM92, C1orf226, CD163, LINC00482, miR-3918, AK5, CCR7, CD200, MICU3, miR-760, SH3PXD2B, miR-760, miR-125b-5p, miR-1299, a miRNA biomarker listed in FIG. 3C or 12B, and a gene biomarker listed in FIG. 3D, 4C, or 12B has been determined in a sample from the subject.

2. The method of claim 1, wherein the biomarker comprises one or more of MMP9, TMEM92, C1orf226, CD163, LINC00482, miR-3918, AK5, CCR7, CD200, MICU3, and miR-760.

3. The method of claim 2, wherein the biomarker comprises one or more of MMP9, TMEM92, C1orf226, CD163, LINC00482, hsa-miR-3918, AK5, CD200, MICU3, and hsa-miR-760.

4. The method of claim 3, wherein the biomarker comprises MMP9, TMEM92, C1orf226, CD163, LINC00482, hsa-miR-3918, AK5, CD200, MICU3, and hsa-miR-760.

5. The method of any one of claims 1-4, wherein the sample from the subject is a globin mRNA depleted sample.

6. The method of any one of claims 1-4, wherein at least MMP9 was determined in a sample from the subject.

7. The method of any one of claims 1-6, wherein at least TMEM92 was determined in a sample from the subject.

8. The method of any one of claims 1-7, wherein at least C1orf226 was determined in a sample from the subject.

9. The method of any one of claims 1-8, wherein at least CD163 was determined in a sample from the subject.

10. The method of any one of claims 1-9, wherein at least LINC00482 was determined in a sample from the subject.

11. The method of any one of claims 1-10, wherein at least miR-3918 was determined in a sample from the subject.

12. The method of any one of claims 1-11, wherein at least AK5 was determined in a sample from the subject.

13. The method of any one of claims 1-12, wherein at least CCR7 was determined in a sample from the subject.

14. The method of any one of claims 1-13, wherein at least CD200 was determined in a sample from the subject.

15. The method of any one of claims 1-14, wherein at least MICU3 was determined in a sample from the subject.

16. The method of any one of claims 1-15, wherein at least miR-760 was determined in a sample from the subject.

17. The method of any one of claims 1-16, wherein the subject has not been diagnosed with or has not been treated for GBM.

18. The method of any one of claims 1-17, wherein the expression levels of the one or more biomarkers in the sample was determined to be i) increased compared to the levels of expression in samples from subjects identified as not having GBM, subjects identified as low risk, or normal tissues or ii) within the range of expression levels in samples of subjects identified as having GBM or identified as high risk.

19. The method of any one of claims 1-18, wherein the expression levels of the one or more biomarkers in the sample was determined to be i) decreased compared to the levels of expression in samples from subjects identified as not having GBM, subjects identified as low risk, or normal tissues or ii) within the range of expression levels in samples of subjects identified as having GBM or identified as high risk.

20. The method of any one of claims 1-19, wherein the expression levels of at least one of the biomarkers in the sample was determined to be i) increased compared to the levels of expression in samples from subjects identified as not having GBM, subjects identified as low risk, or in normal tissues or ii) within the range of expression levels in samples of subjects identified as having GBM or identified as high risk.

21. The method of any one of claims 1-20, wherein the expression levels of at least one of the biomarkers in the sample was determined to be i) decreased compared to the levels of expression in samples from subjects identified as not having GBM, subjects identified as low risk, or in normal tissues or ii) within the range of expression levels in samples of subjects identified as having GBM or identified as high risk.

22. The method of any one of claims 1-21, wherein the expression levels of at least two of the biomarkers in the sample were determined to be i) increased compared to the levels of expression in samples from subjects identified as not having GBM, subjects identified as low risk, or normal tissues or ii) within the range of expression levels in samples of subjects identified as having GBM or identified as high risk.

23. The method of any one of claims 1-22, wherein the expression levels of at least two of the biomarkers in the sample were determined to be i) decreased compared to the levels of expression in samples from subjects identified as not having GBM, subjects identified as low risk, or normal tissues or ii) within the range of expression levels in samples of subjects identified as having GBM or identified as high risk.

24. The method of any one of claims 1-23, wherein the expression levels of at least three of the biomarkers in the sample were determined to be i) increased compared to the levels of expression in samples from subjects identified as not having GBM, subjects identified as low risk, or in normal tissues or ii) within the range of expression levels in samples of subjects identified as having GBM or identified as high risk.

25. The method of any one of claims 1-23, wherein the expression levels of at least three of the biomarkers in the sample were determined to be i) increased compared to the levels of expression in samples from subjects identified as not having GBM, subjects identified as low risk, or in normal tissues or ii) within the range of expression levels in samples of subjects identified as having GBM or identified as high risk.

26. The method of any one of claims 1-25, wherein the expression levels of at least four of the biomarkers in the sample were determined to be i) increased compared to the levels of expression in samples from subjects identified as not having GBM, subjects identified as low risk, or in normal tissues or ii) within the range of expression levels in samples of subjects identified as having GBM or identified as high risk.

27. The method of any one of claims 1-26, wherein the expression levels of at least four of the biomarkers in the sample were determined to be i) decreased compared to the levels of expression in samples from subjects identified as not having GBM, subjects identified as low risk, or in normal tissues or ii) within the range of expression levels in samples of subjects identified as having GBM or identified as high risk.

28. The method of any one of claims 1-27, wherein the expression levels of at least five of the biomarkers in the sample were determined to be i) increased compared to the levels of expression in samples from subjects identified as not having GBM, subjects identified as low risk, or in normal tissues or ii) within the range of expression levels in samples of subjects identified as having GBM or identified as high risk.

29. The method of any one of claims 1-28, wherein the expression levels of at least five of the biomarkers in the sample were determined to be i) decreased compared to the levels of expression in samples from subjects identified as not having GBM, subjects identified as low risk, or in normal tissues or ii) within the range of expression levels in samples of subjects identified as having GBM or identified as high risk.

30. The method of any one of claims 1-29, wherein the expression levels of at least six of the biomarkers in the sample were determined to be i) increased compared to the levels of expression in samples from subjects identified as not having GBM, subjects identified as low risk, or in normal tissues or ii) within the range of expression levels in samples of subjects identified as having GBM or identified as high risk.

31. The method of any one of claims 1-30, wherein the expression levels of at least six of the biomarkers in the sample were determined to be i) decreased compared to the levels of expression in samples from subjects identified as not having GBM, subjects identified as low risk, or in normal tissues or ii) within the range of expression levels in samples of subjects identified as having GBM or identified as high risk.

32. The method of any one of claims 1-31, wherein the expression levels of at least seven of the biomarkers in the sample were determined to be i) increased compared to the levels of expression in samples from subjects identified as not having GBM, subjects identified as low risk, or in normal tissues or ii) within the range of expression levels in samples of subjects identified as having GBM or identified as high risk.

33. The method of any one of claims 1-32, wherein the expression levels of at least seven of the biomarkers in the sample were determined to be i) decreased compared to the levels of expression in samples from subjects identified as not having GBM, subjects identified as low risk, or in normal tissues or ii) within the range of expression levels in samples of subjects identified as having GBM or identified as high risk.

34. The method of any one of claims 1-33, wherein the expression levels of at least eight of the biomarkers in the sample were determined to be i) increased compared to the levels of expression in samples from subjects identified as not having GBM, subjects identified as low risk, or in normal tissues or ii) within the range of expression levels in samples of subjects identified as having GBM or identified as high risk.

35. The method of any one of claims 1-34, wherein the expression levels of at least eight of the biomarkers in the sample were determined to be i) decreased compared to the levels of expression in samples from subjects identified as not having GBM, subjects identified as low risk, or in normal tissues or ii) within the range of expression levels in samples of subjects identified as having GBM or identified as high risk.

36. The method of any one of claims 1-35, wherein the expression levels of at least nine of the biomarkers in the sample were determined to be i) increased compared to the levels of expression in samples from subjects identified as not having GBM, subjects identified as low risk, or in normal tissues or ii) within the range of expression levels in samples of subjects identified as having GBM or identified as high risk.

37. The method of any one of claims 1-36, wherein the expression levels of at least nine of the biomarkers in the sample were determined to be i) decreased compared to the levels of expression in samples from subjects identified as not having GBM, subjects identified as low risk, or in normal tissues or ii) within the range of expression levels in samples of subjects identified as having GBM or identified as high risk.

38. The method of any one of claims 1-37, wherein the expression levels of at least ten of the biomarkers in the sample were determined to be i) increased compared to the levels of expression in samples from subjects identified as not having GBM, subjects identified as low risk, or in normal tissues or ii) within the range of expression levels in samples of subjects identified as having GBM or identified as high risk.

39. The method of any one of claims 1-38, wherein the expression levels of at least ten of the biomarkers in the sample were determined to be i) decreased compared to the levels of expression in samples from subjects identified as not having GBM, subjects identified as low risk, or in normal tissues or ii) within the range of expression levels in samples of subjects identified as having GBM or identified as high risk.

40. The method of any one of claims 1-39, wherein the expression levels of at least eleven of the biomarkers in the sample were determined to be i) increased compared to the levels of expression in samples from subjects identified as not having GBM, subjects identified as low risk, or in normal tissues or ii) within the range of expression levels in samples of subjects identified as having GBM or identified as high risk.

41. The method of any one of claims 1-40, wherein the expression levels of at least eleven of the biomarkers in the sample were determined to be i) decreased compared to the levels of expression in samples from subjects identified as not having GBM, subjects identified as low risk, or in normal tissues or ii) within the range of expression levels in samples of subjects identified as having GBM or identified as high risk.

42. The method of any one of claims 1-41, wherein the expression levels of MMP9, TMEM92, C1orf226, CD163, LINC00482 and/or miR-3918 were determined to be i) increased compared to the levels of expression in samples from subjects identified as not having GBM, subjects identified as low risk, or in normal tissues or ii) within the range of expression levels in samples of subjects identified as having GBM or identified as high risk.

43. The method of any one of claims 1-42, wherein the expression levels of AK5, CCR7, CD200, MICU3, and/or miR-760 were determined to be i) decreased compared to the levels of expression in samples from subjects identified as not having GBM, subjects identified as low risk, or in normal tissues or ii) within the range of expression levels in samples of subjects identified as having GBM or identified as high risk.

44. The method of any one of claims 1-43, wherein the subject is treated for GBM.

45. The method of any one of claim 18-44, wherein the treatment comprises one or more of anticonvulsants, corticosteroids, immunotherapy, surgery, radiotherapy, or chemotherapy.

46. The method of claim 45, wherein the chemotherapy comprises temozolomide.

47. The method of claim 46, wherein the chemotherapy is administered orally or intravenously.

48. The method of any one of claim 1-47, wherein the wherein the sample from the subject comprises a biopsy sample, a serum sample, a tissue sample, a blood sample, a whole blood sample, or a plasma sample.

49. The method of any one of claims 18-48, wherein the normal tissues comprises non-cancerous neural tissues.

50. The method of any one of claims 1-49, wherein the sample from the subject comprises nucleic acids.

51. The method of any one of claims 1-50, wherein the sample from the subject comprises a fractionated blood sample comprising nucleic acids.

52. The method of any one of claims 1-51, wherein the sample or subject comprises a human sample or subject.

53. The method of any one of claims 1-52, wherein the samples from subjects identified as not having GBM or identified as low risk comprises the level of expression of the one or more biomarkers in a blood sample or samples from subjects without GBM.

54. The method of any of claims 1-53, wherein the expression level of no other biomarker in the biological sample was determined.

55. The method of any of claims 1-54, wherein the subject has undergone surgery to resect all or part of the cancer.

56. The method of any one of claims 1-54, wherein the subject has not undergone surgical resection of the tumor.

57. The method of any of claims 1-55, wherein the level of expression of TMEM92 was determined pre-operative and/or post-operative.

58. The method of any of claims 1-55 or 57, wherein the level of expression of C1orf226 was determined pre-operative and/or post-operative.

59. The method of any of claims 1-55 or 57-58, wherein the level of expression of AK5 was determined pre-operative and/or post-operative.

60. The method of any of claims 1-55 or 57-59, wherein the level of expression of MICU3 was determined pre-operative and/or post-operative.

61. The method of any of claims 1-55 or 57-60, wherein the level of expression of miR-3918 was determined pre-operative and/or post-operative.

62. The method of any of claims 1-55 or 57-61, wherein the level of expression of MMP9 was determined pre-operative and/or post-operative.

63. The method of any of claims 1-55 or 57-62, wherein the level of expression of CD163 was determined pre-operative and/or post-operative.

64. The method of any of claims 1-55 or 57-63, wherein the level of expression of LINC00482 was determined pre-operative and/or post-operative.

65. The method of any of claims 1-55 or 57-64, wherein the level of expression of CCR7 was determined pre-operative and/or post-operative.

66. The method of any of claims 1-55 or 57-65, wherein the level of expression of CD200 was determined pre-operative and/or post-operative.

67. The method of any of claims 1-55 or 57-66, wherein the level of expression of miR-760 was determined pre-operative and/or post-operative.

68. A method for evaluating a subject comprising measuring the level of expression of one or more biomarkers selected from MMP9, TMEM92, C1orf226, CD163, LINC00482, miR-3918, AK5, CCR7, CD200, MICU3, miR-760, SH3PXD2B, miR-760, miR-125b-5p, miR-1299, a miRNA biomarker listed in FIG. 3C or 12B, and a gene biomarker listed in FIG. 3D, 4C, or 12B in a sample from the subject.

69. The method of claim 68, wherein the one or biomarkers comprises one or more of MMP9, TMEM92, C1orf226, CD163, LINC00482, miR-3918, AK5, CCR7, CD200, MICU3, and miR-760.

70. The method of claim 69, wherein the one or biomarkers comprises one or more of MMP9, TMEM92, C1orf226, CD163, LINC00482, miR-3918, AK5, CD200, MICU3, and miR-760.

71. The method of claim 70, wherein the biomarkers comprise MMP9, TMEM92, C1orf226, CD163, LINC00482, miR-3918, AK5, CD200, MICU3, and miR-760.

72. The method of any one of claims 68-71, wherein the sample comprises a globin mRNA depleated sample.

73. The method of any one of claims 68-72, wherein the method comprises depleting globin mRNA from the sample.

74. The method of any one of claims 68-73, wherein the subject has not been diagnosed with or identified as high risk for GBM.

75. The method of any one of claims 68-74, wherein the subject has been diagnosed or identified as high risk for GBM.

76. The method of any one of claims 68-75, wherein the sample from the subject comprises a biopsy sample, a serum sample, a tissue sample, a blood sample, a whole blood sample, or a plasma sample.

77. The method of any one of claims 68-76, wherein the sample from the subject comprises nucleic acids.

78. The method of any one of claims 68-77, wherein the sample from the subject comprises a fractionated blood sample comprising nucleic acids.

79. The method of any one of claims 68-78, wherein the sample is from a human.

80. The method of any one of claims 68-79, wherein at least TMEM92 was measured.

81. The method of any one of claims 68-80, wherein at least C1orf226 was measured.

82. The method of any one of claims 68-81, wherein at least AK5 was measured.

83. The method of any one of claims 68-82, wherein at least MICU3 was measured.

84. The method of any one of claims 68-83, wherein at least miR-3918 was measured.

85. The method of any one of claims 68-84, wherein at least MMP9 was measured.

86. The method of any one of claims 68-85, wherein at least CD163 was measured.

87. The method of any one of claims 68-86, wherein at least LINC00482 was measured.

88. The method of any one of claims 68-87, wherein at least CCR7 was measured.

89. The method of any one of claims 68-88, wherein at least CD200 was measured.

90. The method of any one of claims 68-89, wherein at least miR-760 was measured.

91. The method of any of claims 68-90, wherein the expression level of no other biomarker in the biological sample is measured.

92. The method of any of claims 68-91, further comprising comparing the level(s) of expression to a control sample(s) or control level(s) of expression.

93. The method of claim 92, wherein the control sample(s) have expression levels that are representative of expression levels in samples from subjects identified as low risk or of subjects not having GBM.

94. The method of claim 92, wherein the control levels(s) comprise the levels of expression of the one or more biomarkers in non-cancerous neural tissues.

95. The method of claim 92, wherein the control sample(s) have expression levels that are representative of expression levels in samples from subjects identified as high risk or of subjects having GBM.

96. The method of any of claims 68-95, further comprising treating the subject for cancer after measuring the level of expression of the one or more biomarkers.

97. The method of claim 96, wherein the treatment comprises one or more of anticonvulsants, corticosteroids, surgery, radiotherapy, or chemotherapy.

98. The method of claim 97, wherein the chemotherapy comprises temozolomide.

99. The method of claim 97 or 98, wherein the chemotherapy is administered orally or intravenously.

100. The method of any one of claims 68-99, wherein the biomarker is measured prior to surgical resection of the tumor.

101. The method of any one of claims 68-99, wherein the biomarker is measured after surgical resection of the tumor.

102. The method of any of claims 68-101, wherein the level of expression of TMEM92 was measured pre-operative and/or post-operative.

103. The method of any of claims 68-102, wherein the level of expression of C1orf226 was measured pre-operative and/or post-operative.

104. The method of any of claims 68-103, wherein the level of expression of AK5 was measured pre-operative and/or post-operative.

105. The method of any of claims 68-104, wherein the level of expression of MICU3 was measured pre-operative and/or post-operative.

106. The method of any of claims 68-105, wherein the level of expression of miR-3918 was measured pre-operative and/or post-operative.

107. The method of any of claims 68-106, wherein the level of expression of MMP9 was measured pre-operative and/or post-operative.

108. The method of any of claims 68-107, wherein the level of expression of CD163 was measured pre-operative and/or post-operative.

109. The method of any of claims 68-108, wherein the level of expression of LINC00482 was measured pre-operative and/or post-operative.

110. The method of any of claims 68-109, wherein the level of expression of CCR7 was measured pre-operative and/or post-operative.

111. The method of any of claims 68-110, wherein the level of expression of CD200 was measured pre-operative and/or post-operative.

112. The method of any of claims 68-111, wherein the level of expression of miR-760 was measured pre-operative and/or post-operative.

113. A method of prognosing and/or diagnosing a subject for GBM comprising:
a) measuring the level of expression of one or more of MMP9, TMEM92, C1orf226, CD163, LINC00482, miR-3918, AK5, CCR7, CD200, MICU3, miR-760, SH3PXD2B, miR-760, miR-125b-5p, miR-1299, a miRNA biomarker listed in FIG. 3C or 12B, and a gene biomarker listed in FIG. 3D, 4C, or 12B in a sample from the subject;
b) comparing the level(s) of expression to a control sample(s) or control level(s) of expression; and, c) prognosing and/or diagnosing the subject based on the levels of measured expression.

114. The method of claim 113, wherein the biomarker comprises one or more of MMP9, TMEM92, C1orf226, CD163, LINC00482, miR-3918, AK5, CCR7, CD200, MICU3, and miR-760.

115. The method of claim 114, wherein the biomarker comprises one or more of TMEM92, C1orf226, AK5, MICU3, and miR-3918.

116. The method of any one of claims 113-115, wherein at least TMEM92, C1orf226, AK5, MICU3, and miR-3918 were measured in a sample from the subject.

117. The method of any one of claims 113-116, wherein at least MMP9, TMEM92, C1orf226, CD163, LINC00482, miR-3918, AK5, CD200, MICU3, and miR-760 were measured in a sample from the subject.

118. The method of any one of claims 113-117, wherein the sample comprises a globin mRNA dpleated sample.

119. The method of any one of claims 113-118, wherein the method comprises depleting globin mRNA from the sample.

120. The method of any one of claims 113-119, wherein at least MMP9 was measured in a sample from the subject.

121. The method of any one of claims 113-120, wherein at least TMEM92 was measured in a sample from the subject.

122. The method of any one of claims 113-121, wherein at least C1orf226 was measured in a sample from the subject.

123. The method of any one of claims 113-122, wherein at least CD163 was measured in a sample from the subject.

124. The method of any one of claims 113-123, wherein at least LINC00482 was measured in a sample from the subject.

125. The method of any one of claims 113-124, wherein at least miR-3918 was measured in a sample from the subject.

126. The method of any one of claims 113-125, wherein at least AK5 was measured in a sample from the subject.

127. The method of any one of claims 113-126, wherein at least CCR7 was measured in a sample from the subject.

128. The method of any one of claims 113-127, wherein at least CD200 was measured in a sample from the subject.

129. The method of any one of claims 113-128, wherein at least MICU3 was measured in a sample from the subject.

130. The method of any one of claims 113-129, wherein at least miR-760 was measured in a sample from the subject.

131. The method of any one of claims 113-125, wherein the subject has not been diagnosed with or has not been treated for GBM.

132. The method of any one of claims 113-131, wherein the subject is diagnosed as having GBM, prognosed as high risk, and/or treated when the expression levels of MMP9, TMEM92, C1orf226, CD163, LINC00482 and/or miR-3918 were determined to be i) increased compared to the levels of expression in samples from subjects identified as not having GBM, subjects identified as low risk, or in normal tissues or ii) within the range of expression levels in samples of subjects identified as having GBM or identified as high risk.

133. The method of any one of claims 113-132, wherein the subject is diagnosed as having GBM, prognosed as high risk, and/or treated when the expression levels of AK5, CCR7, CD200, MICU3, and/or-miR-760 were determined to be i) decreased compared to the levels of expression in samples from subjects identified as not having GBM, subjects identified as low risk, or in normal tissues or ii) within the range of expression levels in samples of subjects identified as having GBM or identified as high risk.

134. The method of any one of claim 132-133, wherein the treatment comprises one or more of anticonvulsants, corticosteroids, surgery, radiotherapy, or chemotherapy.

135. The method of claim 134, wherein the chemotherapy comprises temozolomide.

136. The method of claim 135, wherein the chemotherapy is administered orally or intravenously.

137. The method of any one of claims 113-131, wherein the subject is diagnosed as not having GBM, prognosed as low risk, and/or not treated when the expression levels of the one or more of MMP9, TMEM92, C1orf226, CD163, LINC00482 and/or miR-3918 in the subject were determined to be i) within range of levels of expression in samples from subjects identified as not having GBM, subjects identified as low risk, or in normal tissues or ii) decreased compared to expression levels in samples of subjects identified as having GBM or identified as high risk.

138. The method of any one of claims 113-131, wherein the subject is diagnosed as not having GBM, prognosed as low risk, and/or not treated when the expression levels of the one or more of AK5, CCR7, CD200, MICU3, and/or-miR-760 in the subject were determined to be i) within range of levels of expression in samples from subjects identified as not having GBM, subjects identified as low risk, or in normal tissues or ii) increased compared to expression levels in samples of subjects identified as having GBM or identified as high risk.

139. The method of any one of claim 113-138, wherein the wherein the sample from the subject comprises a biopsy sample, a serum sample, a tissue sample, a blood sample, a whole blood sample, or a plasma sample.

140. The method of any one of claims 132-139, wherein the normal tissues comprises non-cancerous neural tissues.

141. The method of any one of claims 113-140, wherein the sample from the subject comprises nucleic acids.

142. The method of any one of claims 113-141, wherein the sample from the subject comprises a fractionated blood sample comprising nucleic acids.

143. The method of any of claims 113-142, wherein the expression level of no other biomarker in the biological sample was measured.

144. The method of any one of claims 113-143, wherein the sample or subject is a human sample or subject.

145. The method of any of claims 113-144, wherein the subject has undergone surgery to resect all or part of the cancer.

146. The method of any one of claims 113-144, wherein the subject has not undergone surgical resection of the tumor.

147. The method of any of claims 113-146, wherein the level of expression of TMEM92 was measured pre-operative and/or post-operative.

148. The method of any of claims 113-147, wherein the level of expression of C1orf226 was measured pre-operative and/or post-operative.

149. The method of any of claims 113-148, wherein the level of expression of AK5 was measured pre-operative and/or post-operative.

150. The method of any of claims 113-149, wherein the level of expression of MICU3 was measured pre-operative and/or post-operative.

151. The method of any of claims 113-150, wherein the level of expression of miR-3918 was measured pre-operative and/or post-operative.

152. The method of any of claims 113-151, wherein the level of expression of MMP9 was measured pre-operative and/or post-operative.

153. The method of any of claims 113-152, wherein the level of expression of CD163 was measured pre-operative and/or post-operative.

154. The method of any of claims 113-153, wherein the level of expression of LINC00482 was measured pre-operative and/or post-operative.

155. The method of any of claims 113-154, wherein the level of expression of CCR7 was measured pre-operative and/or post-operative.

156. The method of any of claims 113-155, wherein the level of expression of CD200 was measured pre-operative and/or post-operative.

157. The method of any of claims 113-156, wherein the level of expression of miR-760 was measured pre-operative and/or post-operative.

158. A kit comprising 1, 2, 3, 4, or 5 detection agents for determining expression levels of biomarkers for GBM, wherein the biomarkers comprise one or more MMP9, TMEM92, C1orf226, CD163, LINC00482, miR-3918, AK5, CCR7, CD200, MICU3, miR-760, SH3PXD2B, miR-760, miR-125b-5p, miR-1299, a miRNA biomarker listed in FIG. 3C or 12B, and a gene biomarker listed in FIG. 3D, 4C, or 12B.

159. The kit of claim 158, wherein the kit comprises detection agent for determining expression levels of one or more of MMP9, TMEM92, C1orf226, CD163, LINC00482, miR-3918, AK5, CD200, MICU3, and miR-760.

160. The kit of claim 159, wherein the kit comprises detection agent for determining expression levels of one or more of TMEM92, C1orf226, AK5, MICU3, and miR-3918.

161. The kit of claim 159, wherein the kit comprises detection agents for determining expression levels of MMP9, TMEM92, C1orf226, CD163, LINC00482, miR-3918, AK5, CD200, MICU3, and miR-760.

162. The kit of any one of claims 158-161, wherein the kit further comprises one or more negative or positive control samples and/or control detection agents.

163. The kit of any one of claims 158-162, wherein the kit comprises globin reduction reagents.

164. The kit of claim 163, wherein the globin reduction reagents comprise hemoglobin alpha and beta capture oligos.

165. A method for making and amplifying cDNA comprising a) reverse transcribing the mRNA in a biological sample from a subject; and
b) contacting the sample from a with primers to amplify one or more biomarkers, wherein the biomarkers comprise one or more of MMP9, TMEM92, C1orf226, CD163, LINC00482, miR-3918, AK5, CCR7, CD200, MICU3, miR-760, SH3PXD2B, miR-760, miR-125b-5p, miR-1299, a miRNA biomarker listed in FIG. 3C or 12B, and a gene biomarker listed in FIG. 3D, 4C, or 12B.

166. The method of claim 165, wherein the wherein the biomarkers comprise one or more of comprises detection agent for determining expression levels of one or more of MMP9, TMEM92, C1orf226, CD163, LINC00482, miR-3918, AK5, CD200, MICU3, and miR-760.

167. The method of claim 166, wherein the biomarkers comprise MMP9, TMEM92, C1orf226, CD163, LINC00482, miR-3918, AK5, CD200, MICU3, and miR-760.

168. The method of any one of claims 165-167, wherein the sample is a globin mRNA depleated sample.

169. The method of any one of claims 165-168, wherein the method further comprises depleting globin mRNA from the sample.

170. The method of claim 169, wherein the globin mRNA is depleted prior to reverse transcription.

171. The method of any one of claims 165-170, wherein the sample comprises a a blood sample, a whole blood sample, or a plasma sample.

172. The method of any one of claims 165-171, wherein the sample is from a human subject.


SEQUENCE LISTING (22 sequences)

SEQ ID NO: 1 - DNA, 20 bp, Artificial Sequence
  Synthetic polynucleotide
gagtcaacggatttggtcgt

SEQ ID NO: 2 - DNA, 20 bp, Artificial Sequence
  Synthetic polynucleotide
ttgacagcgacaagaagtgg

SEQ ID NO: 3 - DNA, 20 bp, Artificial Sequence
  Synthetic polynucleotide
agccaaatgtggtctcatcc

SEQ ID NO: 4 - DNA, 20 bp, Artificial Sequence
  Synthetic polynucleotide
gcaggaggtgacactctcaa

SEQ ID NO: 5 - DNA, 20 bp, Artificial Sequence
  Synthetic polynucleotide
ctggcgtgacatgttctgat

SEQ ID NO: 6 - DNA, 25 bp, Artificial Sequence
  Synthetic polynucleotide
gctgctccattggttaaatacttcc

SEQ ID NO: 7 - DNA, 23 bp, Artificial Sequence
  Synthetic polynucleotide
cctaagaatcaggtggggaagga

SEQ ID NO: 8 - DNA, 25 bp, Artificial Sequence
  Synthetic polynucleotide
accatcagtgaagaagattttgctc

SEQ ID NO: 9 - DNA, 22 bp, Artificial Sequence
  Synthetic polynucleotide
ggccctgtaattggaatgagtc

SEQ ID NO: 10 - DNA, 20 bp, Artificial Sequence
  Synthetic polynucleotide
ctctgtgggagcctagatgg

SEQ ID NO: 11 - DNA, 20 bp, Artificial Sequence
  Synthetic polynucleotide
ttgattttggagggatctcg

SEQ ID NO: 12 - DNA, 20 bp, Artificial Sequence
  Synthetic polynucleotide
gccattcacgtcgtccttat

SEQ ID NO: 13 - DNA, 21 bp, Artificial Sequence
  Synthetic polynucleotide
ccaggaagatgatgacgaaga

SEQ ID NO: 14 - DNA, 20 bp, Artificial Sequence
  Synthetic polynucleotide
cgtggtcaactgtccgagaa

SEQ ID NO: 15 - DNA, 24 bp, Artificial Sequence
  Synthetic polynucleotide
cagtctctgaatctccacctcaac

SEQ ID NO: 16 - DNA, 22 bp, Artificial Sequence
  Synthetic polynucleotide
gttgtcaactgccatgctgatg

SEQ ID NO: 17 - DNA, 25 bp, Artificial Sequence
  Synthetic polynucleotide
gacgagaagaattaccagggaaaca

SEQ ID NO: 18 - DNA, 22 bp, Artificial Sequence
  Synthetic polynucleotide
tgtgatgcccttttcttcaggt

SEQ ID NO: 19 - DNA, 23 bp, Artificial Sequence
  Synthetic polynucleotide
cccaagatccaactacgagcttt

SEQ ID NO: 20 - DNA, 20 bp, Artificial Sequence
  Synthetic polynucleotide
ccatagcccttcttaacgcc

SEQ ID NO: 21 - DNA, 18 bp, Artificial Sequence
  Synthetic polynucleotide
cacgaaacagggccgcag

SEQ ID NO: 22 - DNA, 18 bp, Artificial Sequence
  Synthetic polynucleotide
cccggctctgggtctgtg